SEQUENCE LISTING (163 sequences)

SEQ ID NO: 1 - DNA, 3875 bp, Homo sapiens
  Homo sapiens acyl-CoA synthetase long-chain family member 1 (ACSL1)
gggcggggccgcgggagggcggggccggcgcggcgagcgcaccagcagcatcctggctcagccgcggcggtggcgggggcgcaaccagcgggccgaggcggcggcgccagcggcgccttaaatagcatccagagccggcgcggggcagggagtgggctgcagtgacagccggcggcggagcggccggtccacggaggagaattcagcttagagaactatcaacacaggacaatgcaagcccatgagctgttccggtattttcgaatgccagagctggttgacttccgacagtacgtgcgtactcttccgaccaacacgcttatgggcttcggagcttttgcagcactcaccaccttctggtacgccacgagacccaaacccctgaagccgccatgcgacctctccatgcagtcagtggaagtggcgggtagtggtggtgcacgaagatccgcactacttgacagcgacgagcccttggtgtatttctatgatgatgtcacaacattatacgaaggtttccagaggggaatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggatgcctacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccacgctgaagcgatggctcttggactttgcctccaagaggaaagaagcagagcttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctgaccatgcctggagactggaccgcaggccatgttggggccccgatgccgtgcaatttgataaaacttgttgatgtggaagaaatgaattacatggctgccgagggcgagggcgaggtgtgtgtgaaagggccaaatgtatttcagggctacttgaaggacccagcgaaaacagcagaagctttggacaaagacggctggttacacacaggggacattggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccactatcaaggtttagtgtgaagaagaaagctcagaggaaatggcacagttccacaatctcttctcctgctgatggccttcatgttgttaattttgaatacagcaagtgtagggaaggaagcgttcgtgtttgacttgtccattcggggttcttctcataggaatgctagaggaaacagaacactgccttacagtcacctcatgttgcagaccatgtttatggtaatacacactttccaaaatgagccttaaaaattgtaaaggggatactataaatgtgctaagttatttgagacttcctcagtttaaaaagtgggttttaaatcttctgtctccctgtttttctaatcaaggggttaggactttgctatctctgagatgtctgctacttgctgcaaattctgcagctgtctgctgctctaaagagtacagtgcactagagggaagtgttccctttaaaaataagaacaactgtcctggctggagaatctcacaagcggaccagagatctttttaaatccctgctactgtcccttctcacaggcattcacagaacccttctgattcgtaagggttacgaaactcatgttcttctccagtcccctgtggtttctgttggagcataaggtttccagtaagcgggagggcagatccaactcagaaccatgcagataaggagcctctggcaaatgggtgctcatcagaacgcgtggattctctttcatggcagaatgctcttggactcggttctccaggcctgattccccgactccatcctttttcaggggttatttaaaaatctgccttagattctatagtgaagacaagcatttcaagaaagagttacctggatcagccatgctcagctgtgacgcctgaataactgtctactttatcttcactgaaccactcactctgtgtaaaggccaacagatttttaatgtggttttcatatcaaaagatcatgttgggattaacttgcctttttccccaaaaaataaactctcaggcaagcatttctttaaagctattaagggagtatatacttgagtacttattgaaatggacagtaataagcaaatgttcttataatgctacctgatttctatgaaatgtgtttgacaagccaaaattctaggatgtagaaatctggaaagttcatttcctgggattcacttctccagggattttttaaagttaatttgggaaattaacagcagttcactttattgtgagtctttgccacatttgactgaattgagctgtcatttgtacatttaaagcagctgttttggggtctgtgagagtacatgtattatatacaagcacaacagggcttgcactaaagaattgtcattgtaataacactacttggtagcctaacttcatatatgtattcttaattgcacaaaaagtcaataatttgtcaccttggggttttgaatgtttgctttaagtgttggctatttctatgttttataaaccaaaacaaaatttccaaaaacaatgaaggaaaccaaaataaatatttctgcatttcaggtgaaaaaaaaaaaaaaaaaa

SEQ ID NO: 2 - DNA, 1634 bp, Homo sapiens
  Homo sapiens annexin A3 (ANXA3)
gggtggggaagcttagagaccggtgagggagcagagctggggcgcctgtgtacagggatagagcccggcggcagcagggcgcggcttccctttcccggggcctggggccgcaatcaggtggagtcgagaggccggaggaggggcaggaggaaggggtgcggtcgcgatccggacccggagccagcgcggagcacctgcgcccgcggctgacaccttcgctcgcagtttgttcgcagtttactcgcacaccagtttcccccaccgcgctttggattagtgtgatctcagctcaaggcaaaggtgggatatcatggcatctatctgggttggacaccgaggaacagtaagagattatccagactttagcccatcagtggatgctgaagctattcagaaagcaatcagaggaattggaactgatgagaaaatgctcatcagcattctgactgagaggtcaaatgcacagcggcagctgattgttaaggaatatcaagcagcatatggaaaggagctgaaagatgacttgaagggtgatctctctggccactttgagcatctcatggtggccctagtgactccaccagcagtctttgatgcaaagcagctaaagaaatccatgaagggcgcgggaacaaacgaagatgccttgattgaaatcttaactaccaggacaagcaggcaaatgaaggatatctctcaagcctattatacagtatacaagaagagtcttggagatgacattagttccgaaacatctggtgacttccggaaagctctgttgactttggcagatggcagaagagatgaaagtctgaaagtggatgagcatctggccaaacaagatgcccagattctctataaagctggtgagaacagatggggcacggatgaagacaaattcactgagatcctgtgtttaaggagctttcctcaattaaaactaacatttgatgaatacagaaatatcagccaaaaggacattgtggacagcataaaaggagaattatctgggcattttgaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggctttcccaacagctccaccttacttcttctcatactatttaagagaacaagcaaatataaacagcaacttgtgttcctaacaggaattttcattgttctataacaacaacaacaaaagcgattattattttagagcatctcatttataatgtagcagctcataaatgaaattgaaaatggtattaaagatctgcaactactatccaacttatatttctgctttcaaagttaagaatctttatagttctactccattaaatataaagcaagataataaaaattgttgcttttgttaaaagtaaaaaaaaaaaaaaaaaa

SEQ ID NO: 3 - DNA, 926 bp, Homo sapiens
  Homo sapiens cysteine-rich transmembrane module containing 1 (CYSTM1)
gctcgtgctgtgacgcaagcctcgcctcgccccgcgccgcgcgcgttgccagggtgatcaggtgactcccggttcgcggcgctgggagcggccgtgacgtcaggcgcccggctgctcctcacttgctctgagacaggtgcggcaagtctactgcgggctggtccgggctcctcaggttcagacccgaccgttatccagtcggttcgtggagaggagaggtgcactttacaggtccccgatgaaccaagagaaccctccaccatatccaggccctggtccaacggccccatacccaccttatccaccacaaccaatgggtccaggacctatggggggaccctacccacctcctcaagggtacccctaccaaggatacccacagtacggctggcagggtggacctcaggagcctcctaaaaccacagtgtatgtggtagaagaccaaagaagagatgagctaggaccatccacctgcctcacagcctgctggacggctctctgttgctgctgtctctgggacatgctcacctgaccagaccagcccagccgtcctgtcctgccagctctgctgccacctctgacaggtgtgcctgcccccatctcttctgattgctgttaacaaatgactagctttgcacagacacctctaccttcagcactatgggattctagattaatgggggttgctactgtttaattcagtgacttgatctttttaatgtccaaaatccatttcttattgatctttaaagatgtgctaaatgacttttttggccaaaggcttagttgtgaaaaatataatttttaaattatacattcaaggtagtggccaaatgtaacacatcaatcatggaatgatttctctgctaacagccgcctgtatgtttcaataaatttgtccaaagctcaaaaaaaaaaaaaaaaaa

SEQ ID NO: 4 - DNA, 1326 bp, Homo sapiens
  Homo sapiens chromosome 19 open reading frame 59 (C19orf59)
tggacaaatttgcgggctggggaccatggaagtggaggaaatctacaagcaccaggaagtcaagatgcaagcaccagccttcagggacaagaaacagggggtctcagccaagaatcaaggtgcccatgacccagactatgagaatatcaccttggccttcaaaaatcaggaccatgcaaagggtggtcattcacgacccacgagccaagtcccagcccagtgcaggccgccctcagactccacccaggtcccctgctggttgtacagagccatcctgagcctgtacatcctcctggccctggcctttgtcctctgcatcatcctgtcagccttcatcatggtgaagaatgctgagatgtccaaggagctgctgggctttaaaagggagctttggaatgtctcaaactccgtacaagcatgcgaagagagacagaagagaggctgggattccgttcagcagagcatcaccatggtcaggagcaagattgatagattagagacgacattagcaggcataaaaaacattgacacaaaggtacagaaaatcttggaggtgctgcagaaaatgccacagtcctcacctcaataaatgagaggacattgtggcagccaaagccacaacttggaagatggggctgcacctgccaacgaagacgggaaatgaccccccccccccagcctagtgtgaacctgcccctcgtcccacgtatagaaaaacctcgagtcatggtgaatgagtgtctcggagttgctcgtgtgtgtgtacacctgcgtgcgtgtgtgtgcgtgtgtgcgcgtgtgttcgtgtatgtgcgtgtgtgcgtgcgcgtgtgtgtgcattttgcaaagggtggacatttcagtgtatctcccagaaaggtgatgaatgaataggactgagagtcacagtgaatgtggcatgcatgcctgtgtcatgtgacatatgtgagtctcggcatgtcacggtgggtggctgtgtctgagcacctccagcagatgtcactctgagtgtgggtgttggtgacatgcattgcacgggcctgtctccctgtttgtgtaaacatactagagtatactgcggcgtgttttctgtctacccatgtcatggtgggggagatttatctccgtacatgtgggtgtcgccatgtgtgccctgtcactatctgtggctgggtgaacggctgtgtcattatgagtgtgccgagttatgccaccctgtgtgctcagggcacatgcacacagacatttatctctgcactcacattttgtgacttatgaagataaataaagtcaagggaaaacagcgtcaaaaaaaaaaaaaaaaaa

SEQ ID NO: 5 - DNA, 4848 bp, Homo sapiens
  Homo sapiens colony stimulating factor 2 receptor, beta, low-affinity (granulocyte-macrophage) (CSF2RB)
gcctagaggctccagaagaagactggtctctcccaccacacagaggcctggaggaggcagaggccaggagggagaggtcccaagagcctgtgaaatgggtctggcctggctcccagctgggcaggaacacaggacttcaggacactaaggaccctgtcatgcccatggccagcacccaccagtgctggtgcctgcctgtccagagctgaccagggagatggtgctggcccaggggctgctctccatggccctgctggccctgtgctgggagcgcagcctggcaggggcagaagaaaccatcccgctgcagaccctgcgctgctacaacgactacaccagccacatcacctgcaggtgggcagacacccaggatgcccagcggctcgtcaacgtgaccctcattcgccgggtgaatgaggacctcctggagccagtgtcctgtgacctcagtgatgacatgccctggtcagcctgcccccatccccgctgcgtgcccaggagatgtgtcattccctgccagagttttgtcgtcactgacgttgactacttctcattccaaccagacaggcctctgggcacccggctcaccgtcactctgacccagcatgtccagcctcctgagcccagggacctgcagatcagcaccgaccaggaccacttcctgctgacctggagtgtggcccttgggagtccccagagccactggttgtccccaggggatctggagtttgaggtggtctacaagcggcttcaggactcttgggaggacgcagccatcctcctctccaacacctcccaggccaccctggggccagagcacctcatgcccagcagcacctacgtggcccgagtacggacccgcctggccccaggttctcggctctcaggacgtcccagcaagtggagcccagaggtttgctgggactcccagccaggggatgaggcccagccccagaacctggagtgcttctttgacggggccgccgtgctcagctgctcctgggaggtgaggaaggaggtggccagctcggtctcctttggcctattctacaagcccagcccagatgcaggggaggaagagtgctccccagtgctgagggaggggctcggcagcctccacaccaggcaccactgccagattcccgtgcccgaccccgcgacccacggccaatacatcgtctctgttcagccaaggagggcagagaaacacataaagagctcagtgaacatccagatggcccctccatccctcaacgtgaccaaggatggagacagctacagcctgcgctgggaaacaatgaaaatgcgatacgaacacatagaccacacatttgagatccagtacaggaaagacacggccacgtggaaggacagcaagaccgagaccctccagaacgcccacagcatggccctgccagccctggagccctccaccaggtactgggccagggtgagggtcaggacctcccgcaccggctacaacgggatctggagcgagtggagtgaggcgcgctcctgggacaccgagtcggtgctgcctatgtgggtgctggccctcatcgtgatcttcctcaccatcgctgtgctcctggccctccgcttctgtggcatctacgggtacaggctgcgcagaaagtgggaggagaagatccccaaccccagcaagagccacctgttccagaacgggagcgcagagctttggcccccaggcagcatgtcggccttcactagcgggagtcccccacaccaggggccgtggggcagccgcttccctgagctggagggggtgttccctgtaggattcggggacagcgaggtgtcacctctcaccatagaggaccccaagcatgtctgtgatccaccatctgggcctgacacgactccagctgcctcagatctacccacagagcagccccccagcccccagccaggcccgcctgccgcctcccacacacctgagaaacaggcttccagctttgacttcaatgggccctacctggggccgccccacagccgctccctacctgacatcctgggccagccggagcccccacaggagggtgggagccagaagtccccacctccagggtccctggagtacctgtgtctgcctgctggggggcaggtgcaactggtccctctggcccaggcgatgggaccaggacaggccgtggaagtggagagaaggccgagccagggggctgcagggagtccctccctggagtccgggggaggccctgcccctcctgctcttgggccaagggtgggaggacaggaccaaaaggacagccctgtggctatacccatgagctctggggacactgaggaccctggagtggcctctggttatgtctcctctgcagacctggtattcaccccaaactcaggggcctcgtctgtctccctagttccctctctgggcctcccctcagaccagacccccagcttatgtcctgggctggccagtggaccccctggagccccaggccctgtgaagtcagggtttgagggctatgtggagctccctccaattgagggccggtcccccaggtcaccaaggaacaatcctgtcccccctgaggccaaaagccctgtcctgaacccaggggaacgcccggcagatgtgtccccaacatccccacagcccgagggcctccttgtcctgcagcaagtgggcgactattgcttcctccccggcctggggcccggccctctctcgctccggagtaaaccttcttccccgggacccggtcctgagatcaagaacctagaccaggcttttcaagtcaagaagcccccaggccaggctgtgccccaggtgcccgtcattcagctcttcaaagccctgaagcagcaggactacctgtctctgcccccttgggaggtcaacaagcctggggaggtgtgttgagacccccaggcctagacaggcaaggggatggagagggcttgccttccctcccgcctgaccttcctcagtcatttctgcaaagccaaggggcagcctcctgtcaaggtagctagaggcctgggaaaggagatagccttgctccggcccccttgaccttcagcaaatcacttctctccctgcgctcacacagacacacacacacacacgtacatgcacacatttttcctgtcaggttaacttatttgtaggttctgcattattagaactttctagatatactcattccatctccccctcatttttttaatcaggtttccttgcttttgccatttttcttccttcttttttcactgatttattatgagagtggggctgaggtctgagctgagccttatcagactgagatgcggctggttgtgttgaggacttgtgtgggctgcctgtccccggcagtcgctgatgcacatgacatgattctcatctgggtgcagaggtgggaggcaccaggtgggcacccgtgggggttagggcttggaagagtggcacaggactgggcacgctcagtgaggctcagggaattcagactagcctcgattgtcactccgagaaatgggcatggtattgggggtcgggggggcggtgcaagggacgcacatgagagactgtttgggagcttctggggagccctgctagttgtctcagtgatgtctgtgggacctccagtcccttgagaccccacgtcatgtagagaagttaacggcccaagtggtgggcaggctggcgggacctggggaacatcaggagaggagtccagagcccacgtctactgcggaaaagtcaggggaaactgccaaacaaaggaaaatgccccaaaggcatatatgctttagggcctttggtccaaatggcccgggtggccactcttccagatagaccaggcaactctccctcccaccggccacagatgaggggctgctgatctatgcctgggcctgcaccagggattatggttcttttaaatctttgcctttcagatacaggaaaaataatggcattaaattgctttaatttgcattattttagttatccagtttgcacatatttttataggtatcttaggcatcgattggtattttttaactgggccaagcccattaaggtctttcttctgttgggtgctatcattttctgattaagtctttttgactattgacatacagtctttcacagatggtggagtgtttttcccccaaatctgttgtttgtcttataatgttgtatatgaggttttatggtgtatgaatatgaatgcttctgtaatgtcaaacagatccctagtaaactccttcttcacttttactgtcagatttacaaaggtcctcccattgcaaagcagtgtttgtcctaatttatatattgtttttctagttcattttgtgtttccaacttttcatgtaaaattttaattatttttgaatgtgtggatgtgagactgaggtgccttttggtactgaaattctttttccatgtacctgaagtgttacttttgtgatataggaaatccttgtatatatactttattggtccctaggcttcctattttgttaccttgctttctctatggcatccaccattttgattgttctacttttatgatatgttttcataagtggttaagcaagtattctcgttacttttgctcttaaatccctattcattacagcaatgttggtggtcaaagaaaatgataaacaacttgaatgttcaatggtcctgaaatacataacaacattttagtacattgtaaagtagaatcctctgttcataatgaacaagatgaaccaatgtggattagaaagaagtccgagatattaattccaaaatatccagacattgttaaagggaaaaaattgcaataaaatatttgtaacataaaa

SEQ ID NO: 6 - DNA, 6781 bp, Homo sapiens
  Homo sapiens DEAD (Asp-Glu-Ala-Asp) box polypeptide 60-like (DDX60L)
actcaggcgctggctgctgtgtggctcagagcgcccagtccgacgccaggctctgcgctcggtctcctctgctccacctcctccccgcggggcgtgcagctccgaagctctttggacctagcaggttcggcggccgggaggtgccattcacatcaaatgtagccattttagaggtgtcaaaataaatcagccatatttaataaattccagctctactcttctaccctgtagcccaagaagcagcaacgatggggtcaaaggatcatgcagtatttttcagggaaatgacacagttaattttgaatgaaatgccaaaagctgggtattccagcatattaaatgattttgtggaatctaatttttttgtgattgatggagattccttgcttgtcacatgcctgggtgtaaaatcattcaagtggggacagaatctccactttttctatctggttgaatgctatcttgtggatcttctgagtaacggaggacaattcaccatagttttctttaaggatgctgaatatgcatattttgattttcctgaacttctttcattgaggaccgctttaattctccaccttcaacacaatactaacattgatgtgcaaacggagttttctggatgcttatcacaagattggaagttattcttggaacagcattacccgtattttctgatagtttcagaggaaggcctgagtgatttacaaacgtacctttttaacttcctaatcatacattcctggggaatgaaagtcaatgttgtgctttcatcagggcatgaatctgatactctcagattttatgcatatactatggaaagcacagacagaaaccaaactttttccaaggagaatgaaacagtgattcagagtgcatataaaagcctcatacaacacttggaagaaataagggttttagtattagcaactcattttgaacatttgaaatggaatgatatgatggaagaggcgtatcagactctatttctgcttcagcacctatggtcagaaggatcggacatccagcgtgttctctgtgtcacttcatgttcactatccttgagaatgtaccatcgtgtcttagtgcacagtaattgcctatccctgcaggaggtggaagatttctgcagactgcgttgcctctgtgtggcttttcaactccacttacccctttctcagagagcttgttctcgagtcatcacatgctcttggattaggaacagtgattctttcttaaaaatgaacaagtggtgtgaatatttcattttaagcaacttaaacgtttttggatgctggaatctgaatttaaatcatgtttctgacttgtatgatgagcaattgttaaagaatatagccttctactatgaatttgaaagtactcaagaaccacatttgaatttgggagattccattaggagggattatgaagacctgtggaatgttgtgtcacacctggttaaagaatttaacgttggaaagtcttttcctctgagaacaacaagaagacattttcttagacaagagaaatcggtcattcaagaaatctccttggaaaagatgcctagtgtgggctttattccaatgacatctgctgtaattgatgagtttgttggagatatgatgaaggatttgcctattctaaagagtgatgatccggttgttccttcactgtttaaacaaaagacatctgatgaacttttgcactggcatgctcaaagactccttagtgacgactatgacaggatcaaatgtcatgttgatgaacaatctagagatcctcatgttcttgatttcctgaaaaagattcaggattatcagcaattttatgggaaatcgttagaatcaatctctacgaaagtcattgtgactcaaactactcggccaaaggaggattccagtggtgccagtggtgaaatattacagaataccaaaccccaccaaattaccaaaaagagtaagaaaaagtcatttctcaaagaagatcagaacaaagctcagcaaaacgatgatctgctgttttctattgaagaggagatgaagaacaatttacattctggaataaggaaattggaagattatttgacatcatgtgcaagtaattcagtgaaatttggagttgaaatgttaggattaattgcctgctttaaagcatggaaaaaacattgccgaggtgaaggcaaaatttcgaaagatttaagtatagctgttcaaatgatgaaaaggattcattcactcctggagagatacccagaaattttggaagcagaacatcatcaatatatagctaaatgccttaaatatttaggctttaatgatctggcaaactctttggatccaactctgataggagatgacaaaaataagaagaaatattcgattgacattggaccagctcggtttcaactgcaatacatgggccattacttgataagagatgaaagaaaagatcgggatcccagggtccaggattttattcccaacgcatggcagcaggaactcctggatgtggtagataagaatgagtcagcagtgattgttgccccaacgtcctcaggcaaaacctatgcttcctactactgcatggagaaagtgctgagggagagcgatgtcggggtggttgtgtacgttgcacccgcaaagtcccttgttggtcaagtggctgcaactgttgagaatcgttttactaaaacgttgcctgccggcagaactctatgcggtgcttttacaagagattattgtcacaatgtactaaactgtcaggtacttattacagtgccggaatgttttgaaatcctgttgcttgctcctcatcgccaaaaatgggtggaaaggatcagatatgttatatttgatgaggtccattatcttggcagagaagttggagcaaaattttgggagctcctccttgtcattattcgatgtccctttttggttctttcagctaccataaataacccaaatcttctcaccaagtggctgcaatcagtaaaacagtactggaaacaggcagacaagattatggaagagaaatgtatttctgaaaaacaggctgacaaatgtctcaactttctccaagaccattcatataaaaatcaatcatatgaagttagacttgtgctctgtggagagagatacaatgatttagagaagcatatatgttcagtaaaacatgatgatgtttattttgatcattttcatccctgtgctgcgctaacgacagatattattgaaaagtatggattcccacctgatcttaccctcacccctcaagaaagcatccagctttatgataccatggctcaagtctgggaaacttggcccagggctcaggaattgtgtccagaggaattcattctttttaagaataagatagtcattaagaagttggatgctagaaaatatgaagaaaacttaaaggcagaattgacaaattggattaaaaatggccaagtgaagaaggtcaaaagagtactgaagaaccttagtccggattcattgtctagttcaaaagatatggtgaaaatgtttcctcttcttgttgaaaagttaagacaaatggataagttgcctgcaatattttttttgtttaagaatgatgatgtgggaaaaagagctggaagtgtgtgcacttttctggagaagacagagacaaaaagccatccccacactgaatgtcatagttatgtctttgcaatagatgaagtacttgaaaaagtgaggaagacacagaaaaggatcactaaaaaaaacccaaagaaggctgaaaaactggaaagaaaaaaagtgtatagagctgaatatattaatttcctggagaatctgaagattctggaaatttctgaggactgcacgtatgctgatgtcaaagccctacacactgaaattaccaggaataaagactcaactttggagagggtattaccgcgagtgcgatttacaagacacggcaaagaactgaaggctttagcacaaagggggattggatatcatcacagcagcatgtattttaaagaaaaagagtttgttgagatactctttgtaaaagggcttattagggtagtgacagctactgaaacacttgccttagggatccacatgccatgcaaatctgttgtttttgcccaagactcagtctatctggatgctttaaattacagacagatgtctggtcgtgctggaagaagaggtcaagacctgcttggaaatgtgtatttctttgatatcccattgcccaaaataaaaagactccttgcatccagtgttcctgagctgagaggacagttccctctcagcataaccctggtcctgcgactcatgctgctggcttccaagggagatgacccagaggatgccaaggcaaaggtgttgtcagtgctaaagcattcattgctgtcttttaagagacgaagagccatggagactttgaaactttactttttgttttccttgcagctccttatcaaagaggactatttaaataaaaagggtaatccaaagaaatttgcaggacttgcatcatatttgcatggtcatgaaccttcaaatcttgtttttgtaaattttctcaagagaggccttttccataatctctgtaagccagcctggaaaggctcacaacaattttcccaagatgtgatggaaaagctcgtgttagtattggcaaatttgtttggaagaaaatatattccagcaaaattccaaaatgctaatttaagtttttctcagtcaaaggtgatccttgccgaactcccggaggattttaaagctgctttatatgagtataacctggcagtaatgaaggattttgcctccttcctgctgattgcttccaagtcggtgaacatgaaaaaagagcatcaactccctttgtcaagaatcaaattcacaggtaaagaatgtgaagactcccaactcgtgtctcacttgatgagctgcaagaaaggaagagtagccatttcaccatttgtttgtctttcggggaacacagataatgatttgcttcgaccagagactatcaaccaggtcatcctgcgcacagtcggtgttagtggcactcaggctcctctgctgtggccatggaaattagataaccgaggaaggagaatgccactaaatgcatatgtgctcaatttctataaacacaactgcttgacaagattagaccaaaaaaatgggatgcgtatgggacagcttttaaagtgtttgaaagattttgcattcaacattcaggctatcagtgactccttgagtgaactatgtgaaaataagcgtgacaatgtagtcctggcatttaaacaattgagtcaaaccttttatgagaaacttcaagaaatgcaaattcaaatgagtcaaaatcatttagaataacaccatggaaaactttcaagtctgattatgtggtatttatccctttgcaaggagagatataattaagcttacacaatgaaatggaaaaaatgtttgtcttggagtcaaacagaattaaactcagatatcagctctgctattttctaactgaatgactttaagttatgtaatatatctgagctttaacttcatttttggcaaaaccggagtaaaaatgaatacctctagttgttttgaggattaaatgagataatgtaagaaaagtgattgggattgggtggtgacttaatgaacggtagtggtttttttagtagttaatgtatagcaaaattagtttcacattgtcaagttttcaatacatccccaagttaattgaattttaaattaatgatcaataaatcacaaaggacccaaatcaattctgaacaacaatttagttatgtaagaagacttctgagattacaagaaactcactgctgtggactggatgtttgtgccctcccctccaaaatttttatattgaaattctaaccctcaatgtgatggtattaggagatgataggtcatgagggtggagctccttggatgtaattagtgcctttaacagagagacaagagagcttgttctccaatctctgctcactaccactggatgatacaatgggaagatggccatctgcagaccaagaagcaagccctcaacagaactgaatctacttacaccatgatcttgaactttccagcctccaggattgtgagaaatacatgtttgttgtttagccatctagtctgtggttttctgttgaagcagtctgaattgactaaaacagtcacttggagtagttataaaccactttcctgttgaaagcagaacatgctgattcaactgttttgttcaatagcaatgatagattttgtttaagtcccctacactttcttatttctaaatgatcaagagtacacttcctggcagtgattaaggagtgtgtatctaacagaaaaaatatatataccctgtgaacccgaatatggaattcagattgtttctgccctcagtatcatacttaaaaaacaagcatacaaacaaacataagggaacaaacagcaaccataacaaaaacaaacctttaaaggtgcgtttttgctgtgataaatgaatacggtactctgaaggagaaaaaagtttctcaaatgagcttaaactgcaagtgatttaaaaattagagaatataattcttaaagctattgaaagtttcaaccagaaaacctcaagtgaattttgtatgtaaatgaaatcttgaatgtaagttctgtgattctttaagcaaacaattagctgaaaacttggtattgttgtagtttatgtagtaagtgacttggcacccatcagaaaataaagggcattaaattga

SEQ ID NO: 7 - DNA, 1641 bp, Homo sapiens
  Homo sapiens Fc fragment of IgG, high affinity Ib, receptor (CD64) (FCGR1B)
aatatcttgcatgttacagatttcactactcccaccagcttggagacaacatgtggttcttgacaactctgctcctttggggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagttggtggccatcgatctggaccgtcccctgcccacttgctccccgtgagcactgcgtacaaacatccaaaagttcaacaacaccagaactgtgtgtctcatggtatataactcttaaagcaaataaatgaactgacttcaactgggatacatttggaaatgtggtcatcaaagatgacttgaaatgaggcctactctaaagaattcttgaaaaacttacaagtcaagcctagcctgataatcctattacatagtttgaaaaatagtattttatttctcagaacaaggtaaaaaggtgagtgggtgcatatgtacagaagattaagacagagaaacagacagaaagagacacacacacagccaggagtgggtagatttcagggagacaagagggaatagtatagacaataaggaaggaaatagtacttacaaatgactcctaagggactgtgagactgagagggctcacgcctctgtgttcaggatacttagttcatggcttttctctttgactttactaaaagagaatgtctccatacgcgttctaggcatacaagggggtaactcatgatgagaaatggatgtgttattcttgccctctcttttgaggctctctcataacccctctatttctagagacaacaaaaatgttgccagtcctaggcccctgccctgtaggaaggcagaatgtaactgttctttttgtttaacgattaagtccaaatctccaagtgcggcactgcaaagagacgcttcaagtggggagaagcggcgatatcatagagtccagatcttgcctccagagatttgctttaccttcctgattttctggttactaattagcttcaggatacgctgctctcatacttgggctgtagtttggagacaaaatattttcctgccactgtgtaacatagctgaggtaaaaactgaactatgtaaatgactctactaaaagtttagggaaaaaaaacaggaggagtatgacacacacagcaaaaaaaaaaaaaaaaa

SEQ ID NO: 8 - DNA, 2069 bp, Homo sapiens
  Homo sapiens free fatty acid receptor 2 (FFAR2)
atgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccttgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtagcagtttccctggaccttcagaggtcgcctgggttacacaggagctgggaagcctgggagaggcggagcaggaaggctcccatccagattcagaaatccttagacccagcccaggactgcgactttgaaaaaaatgcctttcaccagcttggtatcccttcctgactgaattgtcctactcaaaggagcataagtcagagatgcacgaagaagtagttaggtatagaagcacctgccgggtgtggtggctcatgcctataatcccagaactttgggaggctgaggcaggtggatcacttgaggtcgggagattgagaacatcctggtcaacatgggaaaaccccgtctctactaaaaatacaaaaaaattagctgggcatggtggcacatgcctataatcccagctactctggaggctgaggcaggagaatccttgaacccgggagttggaggttgcagtgagctgagatcacgccactgcactccagcctagcgacagagcaagactccatttaaaaaaaaaaaaaaaaaaaaaaagaagcaccttcaggctggagaagcagcgtagctaacacaagtccagtccttgtgatgtggctggtagttggggatggccaggctgaagcagagagtcctagagaaatctcgatacaagcttcaaagcaacacctagacactgctctagcggttgatcctggagataaaccaacaagagagagatggaagagaaatactaaatgaggtcaaagaagactcagaaaggttctgagcctggagatgagcagggaggcctcagggcttagacctttaatgataggggtttccctgcattggtttgacctgttgcctttttgatgtgctctgtttgttttcatgtgttgtcttgtctcccctgctaaactgggagctgccaggggtctgggtcttatctccttcctccatggtaccccacacaggccaggatgtggtttggtacccagcaatcagagattggcactccctcatacaggggaaagcaacctggtctagcaaattgaaaataaagatgataaaactctgaaaaaaaaaaaaaaaaaa

SEQ ID NO: 9 - DNA, 2019 bp, Homo sapiens
  Homo sapiens formyl peptide receptor 2 (FPR2)
cgatccaatgggaagaagagatccaatggatcctctatcacgaagatattgagataagaaccaatatggatttgcacccactgcatttgcagccttgaggtcataagcatcctcaggaaaatgcaccaggtgctgctggcaagatggaaaccaacttctccactcctctgaatgaatatgaagaagtgtcctatgagtctgctggctacactgttctgcggatcctcccattggtggtgcttggggtcacctttgtcctcggggtcctgggcaatgggcttgtgatctgggtggctggattccggatgacacgcacagtcaccaccatctgttacctgaacctggccctggctgacttttctttcacggccacattaccattcctcattgtctccatggccatgggagaaaaatggccttttggctggttcctgtgtaagttaattcacatcgtggtggacatcaacctctttggaagtgtcttcttgattggtttcattgcactggaccgctgcatttgtgtcctgcatccagtctgggcccagaaccaccgcactgtgagtctggccatgaaggtgatcgtcggaccttggattcttgctctagtccttaccttgccagttttcctctttttgactacagtaactattccaaatggggacacatactgtactttcaactttgcatcctggggtggcacccctgaggagaggctgaaggtggccattaccatgctgacagccagagggattatccggtttgtcattggctttagcttgccgatgtccattgttgccatctgctatgggctcattgcagccaagatccacaaaaagggcatgattaaatccagccgtcccttacgggtcctcactgctgtggtggcttctttcttcatctgttggtttccctttcaactggttgcccttctgggcaccgtctggctcaaagagatgttgttctatggcaagtacaaaatcattgacatcctggttaacccaacgagctccctggccttcttcaacagctgcctcaaccccatgctttacgtctttgtgggccaagacttccgagagagactgatccactccctgcccaccagtctggagagggccctgtctgaggactcagccccaactaatgacacggctgccaattctgcttcacctcctgcagagactgagttacaggcaatgtgaggatggggtcagggatattttgagttctgttcatcctaccctaatgccagttccagcttcatctacccttgagtcatattgaggcattcaaggatgcacagctcaagtatttattcaggaaaaatgcttttgtgtccctgatttggggctaagaaatagacagtcaggctactaaaatattagtgttattttttgttttttgacttctgcctataccctggggtaagtggagttgggaaatacaagaagagaaagaccagtggggatttgtaagacttagatgagatagcgcataataaggggaagactttaaagtataaagtaaaatgtttgctgtaggttttttatagctattaaaaaaaatcagattatggaagttttcttctatttttagtttgctaagagttttctgtttctttttcttacatcatgagtggactttgcattttatcaaatgcattttctacatgtattaagatggtcatattattcttcttcttttatgtaaatcattataaataatgttcattaagttctgaatgttaaactactcttgaattcctggaataaaccacacttagtcctgatgtactttaaatatttatatctcacaggagttggttagaatttctgtgtttatgtttatatactgttatttcactttttctactatccttgctaagttttcatagaaaataaggaacaaagagaaacttgtaatggtctctgaaaaggaattgagaagtaattcctctgattctgttttctggtgttatatctttattaaatattcagaaaaattcaccagtgaaaaaaaaaa

SEQ ID NO: 10 - DNA, 2551 bp, Homo sapiens
  Homo sapiens heat shock 70kDa protein 1B (HSPA1B)
ggaaaacggccagcctgaggagctgctgcgagggtccgcttcgtctttcgagagtgactcccgcggtcccaaggctttccagagcgaacctgtgcggctgcaggcaccggcgtgttgagtttccggcgttccgaaggactgagctcttgtcgcggatcccgtccgccgtttccagcccccagtctcagagcggagcccacagagcagggcaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggttctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctggcggcttcggggctcagggtcccaagggagggtctgggtcaggccctaccattgaggaggtggattaggggcctttgttctttagtatgtttgtctttgaggtggactgttgggactcaaggactttgctgctgttttcctatgtcatttctgcttcagctctttgctgcttcacttctttgtaaagttgtaacctgatggtaattagctggcttcattatttttgtagtacaaccgatatgttcattagaattctttgcatttaatgttgatactgtaagggtgtttcgttccctttaaatgaatcaacactgccaccttctgtacgagtttgtttgttttttttttttttttttttttttgcttggcgaaaacactacaaaggctgggaatgtatgtttttataatttgtttatttaaatatgaaaaataaaatgttaaactttaaaaaaaaaaaaaaaaaaaaaaaaaaaaaaa

SEQ ID NO: 11 - DNA, 733 bp, Homo sapiens
  Homo sapiens interferon induced transmembrane protein 1 (IFITM1)
aaacagcaggaaatagaaacttaagagaaatacacacttctgagaaactgaaacgacaggggaaaggaggtctcactgagcaccgtcccagcatccggacaccacagcggcccttcgctccacgcagaaaaccacacttctcaaaccttcactcaacacttccttccccaaagccagaagatgcacaaggaggaacatgaggtggctgtgctgggggcaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtgaaaaaaaaaaaaaaaaa

SEQ ID NO: 12 - DNA, 678 bp, Homo sapiens
  Homo sapiens interferon induced transmembrane protein 3 (IFITM3)
aggaaaaggaaactgttgagaaaccgaaactactggggaaagggagggctcactgagaaccatcccagtaacccgaccgccgctggtcttcgctggacaccatgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttatcctcacacgcttttctacaatggcattcaataaagtgcacgtgtttctggtgctaaaaaaaaaa

SEQ ID NO: 13 - DNA, 1498 bp, Homo sapiens
  Homo sapiens interleukin 1, beta (IL1B)
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 14 - DNA, 1794 bp, Homo sapiens
  Homo sapiens interleukin 1 receptor antagonist (IL1RN)
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 15 - DNA, 1363 bp, Homo sapiens
  Homo sapiens leukocyte immunoglobulin-like receptor, subfamily A (with TM domain), member 5 (LILRA5)
atgcagctcagcctgggctacacagccaggtgtcagatgtgtctctgctgatctgagtctgcctgtggcatggacctgcatcttccctgaagcatctccagggctgaaaaatcactgaccatggcaccatggtctcatccatctgcacagctgcagccagtgggaggagacgccgtgagccctgccctcatggttctgctctgcctcgggctgagtctgggccccaggacccacgtgcaggcagggaacctctccaaagccaccctctgggctgagccaggctctgtgatcagccgggggaactctgtgaccatccggtgtcaggggaccctggaggcccaggaataccgtctggttaaagagggaagcccagaaccctgggacacacagaacccactggagcccaagaacaaggccagattctccatcccatccatgacagagcaccatgcagggagataccgctgttactactacagccctgcaggctggtcagagcccagcgaccccctggagctggtggtgacaggattctacaacaaacccaccctctcagccctgcccagtcctgtggtgacctcaggagagaacgtgaccctccagtgtggctcacggctgagattcgacaggttcattctgactgaggaaggagaccacaagctctcctggaccttggactcacagctgacccccagtgggcagttccaggccctgttccctgtgggccctgtgacccccagccacaggtggatgctcagatgctatggctctcgcaggcatatcctgcaggtatggtcagaacccagtgacctcctggagattccggtctcaggagcagctgataacctcagtccgtcacaaaacaagtctgactctgggactgcctcacaccttcaggattacgcagtagagaatctcatccgcatgggcatggccggcttgatcctggtggtccttgggattctgatatttcaggattggcacagccagagaagcccccaagctgcagctggaaggtgaacagaagagagaacaatgcaccattgaatgctggagccttggaagcgaatctgatggtcctaggaggttcgggaagaccatctgaggcctatgccatctggactgtctgctggcaatttctttttttctttcttttcttttctttcttttttttttttttttttttttttgagatggagtcttgctctgtcaccaggctggaatgcagtggcgcaatctgggctcactgcaacctccgcctctcgggttcaagtgattctcctgcctcagcctctggcaatttctagagggaggaatgggtgtttgagtgcagagacactggtctggggtgatccatggagga

SEQ ID NO: 16 - DNA, 1780 bp, Homo sapiens
  Homo sapiens leucine-rich alpha-2-glycoprotein 1 (LRG1)
gcagagctaccatgtcctcttggagcagacagcgaccaaaaagcccagggggcattcaaccccatgtttctagaactctgttcctgctgctgctgttggcagcctcagcctggggggtcaccctgagccccaaagactgccaggtgttccgctcagaccatggcagctccatctcctgtcaaccacctgccgaaatccccggctacctgccagccgacaccgtgcacctggccgtggaattcttcaacctgacccacctgccagccaacctcctccagggcgcctctaagctccaagaattgcacctctccagcaatgggctggaaagcctctcgcccgaattcctgcggccagtgccgcagctgagggtgctggatctaacccgaaacgccctgaccgggctgcccccgggcctcttccaggcctcagccaccctggacaccctggtattgaaagaaaaccagctggaggtcctggaggtctcgtggctacacggcctgaaagctctggggcatctggacctgtctgggaaccgcctccggaaactgccccccgggctgctggccaacttcaccctcctgcgcacccttgaccttggggagaaccagttggagaccttgccacctgacctcctgaggggtccgctgcaattagaacggctacatctagaaggcaacaaattgcaagtactgggaaaagatctcctcttgccgcagccggacctgcgctacctcttcctgaacggcaacaagctggccagggtggcagccggtgccttccagggcctgcggcagctggacatgctggacctctccaataactcactggccagcgtgcccgaggggctctgggcatccctagggcagccaaactgggacatgcgggatggcttcgacatctccggcaacccctggatctgtgaccagaacctgagcgacctctatcgttggcttcaggcccaaaaagacaagatgttttcccagaatgacacgcgctgtgctgggcctgaagccgtgaagggccagacgctcctggcagtggccaagtcccagtgagaccaggggcttgggttgagggtggggggtctggtagaacactgcaacccgcttaacaaataatcctgcctttggccgggtgcgggggctcacgcctgtaatcccagcactttgggaggcccaggtgggcggatcacgaggtcaggagatcgagaccatcttggctaacatggtgaaaccctgtctctactaaaaatataaaaaattagccaggcgtggtggtgggcacctgtagtcccagcaactcgggaggctgaggcaggagaatggcgtgaacttgggaggcggagcttgcggtgagccaagatcgtgccactgcactctagcctgggcgacagagcaagactgtctcaaaaaaattaaaattaaaattaaaaacaaataatcctgccttttacaggtgaaactcggggctgtccatagcggctgggaccccgtttcatccatccatgcttcctagaacacacgatgggctttccttacccatgcccaaggtgtgccctccgtctggaatgccgttccctgtttcccagatctcttgaactctgggttctcccagccccttgtccttccttccagctgagccctggccacactggggctgcctttctctgactctgtcttccccaagtcagggggctctctgagtgcagggtctgatgctgagtcccacttagcttggggtcagaaccaaggggtttaataaataacccttgaaaactgga

SEQ ID NO: 17 - DNA, 4107 bp, Homo sapiens
  Homo sapiens myeloid cell leukemia sequence 1 (BCL2-related) (MCL1)
gcgcaaccctccggaagctgccgcccctttccccttttatgggaatactttttttaaaaaaaaagagttcgctggcgccaccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaatacagggaaaaaaaaaaaaaaaaaa

SEQ ID NO: 18 - DNA, 6551 bp, Homo sapiens
  Homo sapiens NLR family, apoptosis inhibitory protein (NAIP)
tggcacagatctccagaaacccttgtaatttcctgagtgacaggggtgatagaaacatcttttattagaatacttggtcttggttcctgacacaagagcttctaagacctttggaatctccaagtgataagagtgtatgacagtgagctaactggtggctgggatcctttagacaacttcaggatgggggctatcccctgaaagactaaggcatgattagaggtctgggatttgcagccccacgcctcgacctccagagagggtaaaagggctggagattgattaaccaccagttgccagtgatttaaccaatcatgcctaagtgatggcacctccattaaaaaataaaccacaggtttggagagctttcggtttggttaaccccaaccacataccaagaaggcgatgcacctcaaactgcatgaagacaaaaggtcctgtgctcacctgggacccttctggacgttgccctgtgtacctcttcgactgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcgtccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcgtgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattgccaaagtagcaatcagtggaggtttccagaaacttgagaacctaaagctttcaatcaatcacaagattacagaggaaggatacagaaatttctttcaagcactggacaacatgccaaacttgcaggagttggacatctccaggcatttcacagagtgtatcaaagctcaggccacaacagtcaagtctttgagtcaatgtgtgttacgactaccaaggctcattagactgaacatgttaagttggctcttggatgcagatgatattgcattgcttaatgtcatgaaagaaagacatcctcaatctaagtacttaactattctccagaaatggatactgccgttctctccaatcattcagaaataaaagattcagctaaaaactgctgaatcaataatttgtcttggggcatattgaggatgtaaaaaaagttgttgattaatgctaaaaaccaaattatccaaaattattttattaaatattgcatacaaaagaaaatgtgtaaggcttgctaaaaaacaaaacaaaacaaaacacagtcctgcatactcaccaccaagctcaagaaataaatcatcaccaatacctttgaggtccctgagtaatccaccccagctaaaggcaaacccttcaatcaagtttatacagcaaaccctccattgtccatggtcaacagggaaggggttggggacaggtctgccaatctatctaaaagccacaatatggaagaagtattcaatttatataataaatggctaacttaacggttgaatcactttcatacatggatgaaacgggtttaacacaggatccacatgaatcttctgtgggccaagagatgttccttaatccttgtagaacctgttttctatattgaactagctttggtacagtagagttaacttactttccatttatccactgccaatataaagaggaaacaggggttagggaaaaatgacttcattccagaggcttctcagagttcaacatatgctataatttagaattttcttatgaatccactctacttgggtagaaaatattttatctctagtgattgcatattatttccatatcatagtatttcatagtattatatttgatatgagtgtctatatcaatgtcagtgtccagaatttcgttcctaccagttaagtagttttctgaacggccagaagaccattcgaaattcatgatactactataagttggtaaacaaccatacttttatcctcatttttattctcactaagaaaaaagtcaactcccctccccttgcccaagtatgaaatatagggacagtatgtatggtgtggtctcatttgtttagaaaaccacttatgactgggtgcggtggctcacacctgtaatcccagcactttgggaggctgaggcgggcgaatcatttgaggtgaggaattcgagaccagcctggccagcatggtgaaaccccatctctactaaaaatacaaaaattagccaggtgtggtggcacatgcctgtagtcccagccactagggcggctgagacgcaagacttgcttgaacccgggaggcagaggttgcagtgagccaagatggcgccactgcattccagcctgggcaacagagcaagaccctgtctgtctcaaaacaaaaaacaaaaccacttatattgctagctacattaagaatttctgaatatgttactgagcttgcttgtggtaaccatttataatatcagaaagtatatgtacaccaaaacatgttgaacatccatgttgtacaactgaaatataaataattttgtcaattatacctaaataaaactggaaaaaaatttctggaagtttatatctaaaaatgttaatagtgcgtacctctaggaagtgggcctggaagccattcttacttttcagtctctcccattctgtactgttttttgttttactttcgtgcctgcattatttttctatttaaaacaaaaataaatctagtttagcact

SEQ ID NO: 19 - DNA, 2104 bp, Homo sapiens
  Homo sapiens nuclear factor, interleukin 3 regulated (NFIL3)
acgtagcgcggcgctcggaactgacctactaacacacatctctccgcgcggccacggcgcccgcggacccggcgcgcccgcccgcctcccgcgccgcgccctcgccgccgcccgcctcccgccgcggccccggaggcccggcccggcccgagccccgagcgccggcggcccgactcccggccgcccctttctttctcctcgccggcccgagagcaggaacacgataacgaaggaggcccaacttcattcaataaggagcctgacggatttatcccagacggtagaacaaaaggaagaatattgatggattttaaaccagagtttttaaagagcttgagaatacggggaaattaatttgttctcctacacacatagatagggtaaggttgtttctgatgcagctgagaaaaatgcagaccgtcaaaaaggagcaggcgtctcttgatgccagtagcaatgtggacaagatgatggtccttaattctgctttaacggaagtgtcagaagactccacaacaggtgaggagctgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagtttcaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgtcatttgcaatagagcagtccattttgtattatgctgaattttcactggacctgtgatgtcatttcactgtgatgtgcacatgttgtctgtttggtgtctttttgtgcacagattatgatgaagattagattgtgttatcactctgcctgtgtatagtcagatagtccatgcgaaggctgtatatattgaacattatttttgttgttctattataaagtgtgtaagttaccagtttcaataaaggattggtgacaaacacagaaaaaaaaaaaaaaaaaaaaa

SEQ ID NO: 20 - DNA, 1782 bp, Homo sapiens
  Homo sapiens 5′-nucleotidase, cytosolic III (NT5C3)
cgtgatgctctgggatcccgcgcttccgagactcgcagtctacgcgagctgcctgtttttttcctgcttggacgcgcatgagggccccgtccatggaccgcgcggccgtggcgagggtgggcgcggtagcgagcgccagcgtgtgcgccctggtggcgggggtggtgctggctcagtacatattcaccttgaagaggaagacggggcggaagaccaagatcatcgagatgaagattggataaccaagaaatgactaatcaagagtctgccgtacatgtgaaaatgatgccagaattccagaaaagttcagttcgaatcaagaaccctacaagagtagaagaaattatctgtggtcttatcaaaggaggagctgccaaacttcagataataacggactttgatatgacactcagtagattttcatataaagggaaaagatgcccaacatgtcataatatcattgacaactgtaagctggttacagatgaatgtagaaaaaagttattgcaactaaaggaaaaatattacgctattgaagttgatcctgttcttactgtagaagagaagtacccttatatggtggaatggtatactaaatcacatggtttgcttgttcagcaagctttaccaaaagctaaacttaaagaaattgtggcagaatctgacgttatgctcaaagaaggatatgagaatttctttgataagctccaacaacatagcatccccgtgttcatattttcggctggaatcggcgatgtactagaggaagttattcgtcaagctggtgtttatcatcccaatgtcaaagttgtgtccaattttatggattttgatgaaactggggtgctcaaaggatttaaaggagaactaattcatgtatttaacaaacatgatggtgccttgaggaatacagaatatttcaatcaactaaaagacaatagtaacataattcttctgggagactcccaaggagacttaagaatggcagatggagtggccaatgttgagcacattctgaaaattggatatctaaatgatagagtggatgagcttttagaaaagtacatggactcttatgatattgttttagtacaagatgaatcattagaagtagccaactctattttacagaagattctataaacaagcattctccaagaagacctctctcctgtgggtgcaattgaactgttcatccgttcatcttgctgagagacttatttataatatatccttactctcgaagtgttccctttgtataactgaagtattttcagatatggtgaatgcattgactggaagctccttttctccacctctctcaacacactcctcaccgtatcttttaacccatttaaaaaaaaaaaaaagctaaaattagaaaaataactccctacttttccaaagtgaattttgtagtttaatgttatcatgcagcttttgaggagtcttttacactgggaaagtttgtagaaattttaaaataagttttatgaaatggtgaaataatatgcatgattttaagtattgccatttttgtaatttgggttattatgctgatggtatcaccatctcttgaaattgtgttaggtttggttattttgtctggggaaaaaatatttactggaaaagactagcagttagtgttggaaaaacctggtggtgtttacaatgttgctaatcattacaaaacattctatattgaagcactgataataaatatgaaatgcaaaacctttttaattctatggtcaaaactaaaaaaaaaaaaaaaaaaaaaaa

SEQ ID NO: 21 - DNA, 4224 bp, Homo sapiens
  Homo sapiens 6-phosphofructo-2-kinase/fructose- 2,6-biphosphatase 3 (PFKFB3)
ctttcccctccctcgcccgccccgccgcccgcaggcgccccgagtcgcggggctgccgcttggacgtcgtcctgtctgggtgtcgcgggccggccccgcggggagcgcccccggcgcgatgcccttcaggaaagcctgtgggccaaagctgaccaactcccccaccgtcatcgtcatggtgggcctccccgcccggggcaagacctacatctccaagaagctgactcgctacctcaactggattggcgtccccacaaaagtgttcaacgtcggggagtatcgccgggaggctgtgaagcagtacagctcctacaacttcttccgccccgacaatgaggaagccatgaaagtccggaagcaatgtgccttagctgccttgagagatgtcaaaagctacctggcgaaagaagggggacaaattgcggttttcgatgccaccaatactactagagagaggagacacatgatccttcattttgccaaagaaaatgactttaaggcgtttttcatcgagtcggtgtgcgacgaccctacagttgtggcctccaatatcatggaagttaaaatctccagcccggattacaaagactgcaactcggcagaagccatggacgacttcatgaagaggatcagttgctatgaagccagctaccagcccctcgaccccgacaaatgcgacagggacttgtcgctgatcaaggtgattgacgtgggccggaggttcctggtgaaccgggtgcaggaccacatccagagccgcatcgtgtactacctgatgaacatccacgtgcagccgcgtaccatctacctgtgccggcacggcgagaacgagcacaacctccagggccgcatcgggggcgactcaggcctgtccagccggggcaagaagtttgccagtgctctgagcaagttcgtggaggagcagaacctgaaggacctgcgcgtgtggaccagccagctgaagagcaccatccagacggccgaggcgctgcggctgccctacgagcagtggaaggcgctcaatgagatcgacgcgggcgtctgtgaggagctgacctacgaggagatcagggacacctaccctgaggagtatgcgctgcgggagcaggacaagtactattaccgctaccccaccggggagtcctaccaggacctggtccagcgcttggagccagtgatcatggagctggagcggcaggagaatgtgctggtcatctgccaccaggccgtcctgcgctgcctgcttgcctacttcctggataagagtgcagaggagatgccctacctgaaatgccctcttcacaccgtcctgaaactgacgcctgtcgcttatggctgccgtgtggaatccatctacctgaacgtggagtccgtctgcacacaccgggagaggtcagaggatgcaaagaagggacctaacccgctcatgagacgcaatagtgtcaccccgctagccagccccgaacccaccaaaaagcctcgcatcaacagctttgaggagcatgtggcctccacctcggccgccctgcccagctgcctgcccccggaggtgcccacgcagctgcctggacaaaacatgaaaggctcccggagcagcgctgactcctccaggaaacactgaggcagacgtgtcggttccattccatttccatttctgcagcttagcttgtgtcctgccctccgcccgaggcaaaacgtatcctgaggacttcttccggagagggtggggtggagcagcgggggagccttggccgaagagaaccatgcttggcaccgtctgtgtcccctcggccgctggacaccagaaagccacgtgggtccctggcgccctgcctttagccgtggggcccccacctccactctctgggtttcctaggaatgtccagcctcggagaccttcacaaagccttgggagggtgatgagtgctggtcctgacaggaggccgctggggacactgtgctgttttgtttcgtttctgtgatctcccggcacgtttggagctgggaagaccacactggtggcagaatcctaaaattaaaggaggcaggctcctagttgctgaaagttaaggaatgtgtaaaacctccacgtgactgtttggtgcatcttgacctgggaagacgcctcatgggaacgaacttggacaggtgttgggttgaggcctcttctgcaggaagtccctgagctgagacgcaagttggctgggtggtccgcaccctggctctcctgcaggtccacacaccttccaggcctgtggcctgcctccaaagatgtgcaagggcaggctggctgcacggggagagggaagtattttgccgaaatatgagaactggggcctcctgctcccagggagctccagggcccctctctcctcccacctggacttggggggaactgagaaacactttcctggagctgctggcttttgcacttttttgatggcagaagtgtgacctgagagtcccaccttctcttcaggaacgtagatgttggggtgtcttgccctggggggcttggaacctctgaaggtggggagcggaacacctggcatccttccccagcacttgcattaccgtccctgctcttcccaggtggggacagtggcccaagcaaggcctcactcgcagccacttcttcaagagctgcctgcacactgtcttggagcatctgccttgtgcctggcactctgccggtgccttgggaaggtcggaagagtggactttgtcctggccttcccttcatggcgtctatgacacttttgtggtgatggaaagcatgggacctgtcgtctcagcctgttggtttctcctcattgcctcaaaccctggggtaggtgggacggggggtctcgtgcccagatgaaaccatttggaaactcggcagcagagtttgtccaaatgacccttttcaggatgtctcaaagcttgtgccaaaggtcacttttctttcctgccttctgctgtgagccctgagatcctcctcccagctcaagggacaggtcctgggtgagggtgggagatttagacacctgaaactgggcgtggagagaagagccgttgctgtttgttttttgggaagagcttttaaagaatgcatgtttttttcctggttggaattgagtaggaactgaggctgtgcttcaggtatggtacaatcaagtgggggattttcatgctgaaccattcaagccctccccgcccgttgcacccactttggctggcgtctgctggagaggatgtctctgtccgcattcccgtgcagctccaggctcgcgcagttttctctctctccctggatgttgagtctcatcagaatatgtgggtagggggtggacgtgcacgggtgcatgattgtgcttaacttggttgtatttttcgatttgacatggaaggcctgttgctttgctcttgagaatagtttctcgtgtccccctcgcaggcctcattctttgaacatcgactctgaagtttgatacagataggggcttgatagctgtggtcccctctcccctctgactacctaaaatcaatacctaaatacagaagccttggtctaacacgggacttttagtttgcgaagggcctagatagggagagaggtaacatgaatctggacagggagggagatactatagaaaggagaacactgcctactttgcaagccagtgacctgccttttgaggggacattggacgggggccgggggcgggggttgggtttgagctacagtcatgaacttttggcgtctactgattcctccaactctccaccccacaaaataacggggaccaatatttttaactttgcctatttgtttttgggtgagtttcccccctccttattctgtcctgagaccacgggcaaagctcttcattttgagagagaagaaaaactgtttggaaccacaccaatgatatttttctttgtaatacttgaaatttatttttttattattttgatagcagatgtgctatttatttatttaatatgtataaggagcctaaacaatagaaagctgtagagattgggtttcattgttaattggtttgggagcctcctatgtgtgacttatgacttctctgtgttctgtgtatttgtctgaattaatgacctgggatataaagctatgctagctttcaaacaggagatgcctttcagaaatttgtatattttgcagttgccagaccaataaaatacctggttgaaatacatggacgaagtaaa

SEQ ID NO: 22 - DNA, 2228 bp, Homo sapiens
  Homo sapiens phospholipid scramblase 1 (PLSCR1)
caccggacaaacgtctctggagtctctccaatgagcaagaaagcaagtcgggggtaggggaggggcctcacaccagggggtgggcgcagtccctcctccagctccttcaccctccagtagtctcgtgggtccccgagcgccagcgcgggaaccgggaaaaggaaaccgtgttgtgtacgtaagattcaggaaacgaaaccaggagccgcgggtgttggcgcaaaggttactcccagacccttttccggctgacttctgagaaggttgcgcagcagctgtgcccggcagtctagaggcgcagaagaggaagccatcgcctggccccggctctctggaccttgtctcgctcgggagcggaaacagcggcagccagagaactgttttaatcatggacaaacaaaactcacagatgaatgcttctcacccggaaacaaacttgccagttgggtatcctcctcagtatccaccgacagcattccaaggacctccaggatatagtggctaccctgggccccaggtcagctacccacccccaccagccggccattcaggtcctggcccagctggctttcctgtcccaaatcagccagtgtataatcagccagtatataatcagccagttggagctgcaggggtaccatggatgccagcgccacagcctccattaaactgtccacctggattagaatatttaagtcagatagatcagatactgattcatcagcaaattgaacttctggaagttttaacaggttttgaaactaataacaaatatgaaattaagaacagctttggacagagggtttactttgcagcggaagatactgattgctgtacccgaaattgctgtgggccatctagaccttttaccttgaggattattgataatatgggtcaagaagtcataactctggagagaccactaagatgtagcagctgttgttgtccctgctgccttcaggagatagaaatccaagctcctcctggtgtaccaataggttatgttattcagacttggcacccatgtctaccaaagtttacaattcaaaatgagaaaagagaggatgtactaaaaataagtggtccatgtgttgtgtgcagctgttgtggagatgttgattttgagattaaatctcttgatgaacagtgtgtggttggcaaaatttccaagcactggactggaattttgagagaggcatttacagacgctgataactttggaatccagttccctttagaccttgatgttaaaatgaaagctgtaatgattggtgcctgtttcctcattgacttcatgttttttgaaagcactggcagccaggaacaaaaatcaggagtgtggtagtggattagtgaaagtctcctcaggaaatctgaagtctgtatattgattgagactatctaaactcatacctgtatgaattaagctgtaaggcctgtagctctggttgtatacttttgcttttcaaattatagtttatcttctgtataactgatttataaaggtttttgtacattttttaatactcattgtcaatttgagaaaaaggacatatgagtttttgcatttattaatgaaacttcctttgaaaaactgctttgaattatgatctctgattcattgtccattttactaccaaatattaactaaggccttattaatttttatataaattatatcttgtcctattaaatctagttacaatttatttcatgcataagagctaatgttattttgcaaatgccatatattcaaaaaagctcaaagataattttctttactattatgttcaaataatattcaatatgcatattatctttaaaaagttaaatgtttttttaatcttcaagaaatcatgctacacttaacttctcctagaagctaatctataccataatattttcatattcacaagatattaaattaccaattttcaaattattgttagtaaagaacaaaatgattctctcccaaagaaagacacattttaaatactccttcactctaaaactctggtattataacttttgaaagttaatatttctacatgaaatgtttagctcttacactctatccttcctagaaaatggtaattgagattactcagatattaattaaatacaatatcatatatatattcacagagtataaacctaaataatgatctattagattcaaatatttgaaataaaaacttgatttttttgtaaaaaaaaaaaaaaaaaaa

SEQ ID NO: 23 - DNA, 1550 bp, Homo sapiens
  Homo sapiens prokineticin 2 (PROK2)
gccggcgtgagtcacggcggggctagcctttataacggcccggaggctcgcgggagccgccgcgcccgtccgcccgccgctccgcgctccacccagcgcacccgggccccgcgcccccaactgcctccggcggccgcccagtcccgagggcgccatgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccatgtctgccaggcttggcctgtttacggacttcatttaaccgatttatttgtttagcccaaaagtaatcgctctggagtagaaaccaaatgtgaatagccacatcttacctgtaaagtcttacttgtgattgtgccaaacaaaaaatgtgccagaaagaaatgctcttgcttcctcaactttccaagtaacatttttatctttgatttgtaaatgatttttttttttttttatcgaaagagaattttacttttggatagaaatatgaagtgtaaggcattatggaactggttcttatttccctgtttgtgttttggtttgatttggcttttttcttaaatgtcaaaaacgtacccattttcacaaaaatgaggaaaataagaatttgatattttgttagaaaaacttttttttttttttctcaccaccccaagccccatttgtgccctgccacacaaatacacctacagcttttggtcccttgcctcttccacctcaaagaatttcaaggctcttaccttactttatttttgtccatttctcttccctcctcttgcattttaaagtggagggtttgtctctttgagtttgatggcagaatcactgatgggaatccagctttttgctggcatttaaatagtgaaaagagtgtatatgtgaacttgacactccaaactcctgtcatggcacggaagctaggagtgctgctggacccttcctaaacctgtcactcaagaggacttcagctctgctgttgggctggtgtgtggacagaaggaatggaaagccaaattaatttagtccagatttctaggtttgggtttttctaaaaataaaagattacatttacttcttttactttttataaagttttttttccttagtctcctacttagagatattctagaaaatgtcacttgaagaggaagtatttattttaatctggcacaacactaattaccatttttaaagcagtattaagttgtaatttaaaccttgtttgtaactgaaaggtcgattgtaatggattgccgtttgtacctgtatcagtattgctgtgtaaaaattctgtatcagaataataacagtactgtatatcatttgatttattttaatattatatccttatttttgtca

SEQ ID NO: 24 - DNA, 1632 bp, Homo sapiens
  Homo sapiens RAB24, member RAS oncogene family (RAB24)
gaccttgcggccccgccccctcgccctctagccccctcccgcgggagtcgcggcgctgcgggtaggagccgggttgcgggagaccccaggttcggttgggattcccagccagaacggagcttaagccgggcaggcgagcgaatgacggagtagcgagctgcacggcggcgtgctgcgctgttgaggacgctgtcccgcgcgctcccaggccgccccgaggcttggggtcttcgaaggataatcggcgcccggggccgaacagcgggggcacacggggcgctgccgaagtgcaaggccacggccagagctcgagcccgacgcgctgtctggagtcgtaggttggcgccgtttggggtcggggtctgaggcttgggcgctgcctgggccgagcggagatcggggtttgcctcccgtccccgctcaggaccctgacgtggctgaagcggccccgggagcatgagcgggcagcgcgtggacgtcaaggtggtgatgctgggcaaggagtacgtgggcaagactagcctggtggagcgctacgtgcacgaccgctttctggtggggccttatcagaacaccatcggggccgccttcgtggccaaggtgatgtcggtcggagaccggactgtgacattaggtatttgggacacagcaggctctgagcgctatgaggccatgagtagaatctactatcggggtgccaaggctgccatcgtctgctatgacctcacagacagcagcagctttgagcgagcaaagttctgggtgaaggaactgcgcagcctagaggagggctgccaaatctacttatgtggcaccaagagtgacctgctggaagaagaccggaggcgtcgacgtgtggacttccacgacgtccaggactatgcagacaatatcaaagctcagctctttgaaacatccagcaagacaggccagagtgtggacgagctcttccagaaagtggcagaggattacgtcagtgtggctgccttccaggtgatgacagaggacaagggcgtggatctgggccagaagccaaacccctacttctacagctgttgtcatcactgagtcagcactcacctggcctgggggaattaaaggaattccccgtaagggctggacccagctcctttctgggcttgggtagtcaaatgtctgagctaccccaggtcctcatgtcagcagagtggcgcctgcctgtgctggcccatggaacggagacagcattgggctgactgtgggcatgaggagggataaggctgatttggaccccaggcttctgccctggacagcacttgtgtctgcagattatttaagtggcttttgatctgtaaataaaatcagtgcactgtgcatcacacccagcccctttccctgctgtgtggattaggtgtcaagacacctagttcttcctggggccacccggctggcctcactgcttatattaaggctcctcccaactctcattttcctttggaaaacaagacttttttccccatggttaccgctgagatactggggctgtagtagtataaaagctcacagttccttctgagtgctgaaaagagtgcatgagttgcttcgaaataaaagggtcaagcattcctacctgagacaggttaaaaaaaaaaa

SEQ ID NO: 25 - DNA, 466 bp, Homo sapiens
  Homo sapiens S100 calcium binding protein A12 (S100A12)
accactgctggctttttgctgtagctccacattcctgtgcattgaggggttaacattaggctgggaagatgacaaaacttgaagagcatctggagggaattgtcaatatcttccaccaatactcagttcggaaggggcattttgacaccctctctaagggtgagctgaagcagctgcttacaaaggagcttgcaaacaccatcaagaatatcaaagataaagctgtcattgatgaaatattccaaggcctggatgctaatcaagatgaacaggtcgactttcaagaattcatatccctggtagccattgcgctgaaggctgcccattaccacacccacaaagagtaggtagctctctgaaggctttttacccagcaatgtcctcaatgagggtcttttctttccctcaccaaaacccagccttgcccgtggggagtaagagttaataaacacactcacgaaaagtt

SEQ ID NO: 26 - DNA, 2442 bp, Homo sapiens
  Homo sapiens selectin L (SELL)
aggaggaaggggagggaaaaggggaggaggaggaggatgtgagactgggttagagaaatgaaagaaagcaaggctttctgttgacattcagtgcagtctacctgcagcacagcacactccctttgggcaaggacctgagacccttgtgctaagtcaagaggctcaatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaaatcgcccttggtgaaagaaaattcttggaatactaaaaatcatgagatcctttaaatccttccatgaaacgttttgtgtggtggcacctcctacgtcaaacatgaagtgtgtttccttcagtgcatctgggaagatttctacctgaccaacagttccttcagcttccatttcgcccctcatttatccctcaacccccagcccacaggtgtttatacagctcagctttttgtcttttctgaggagaaacaaataagaccataaagggaaaggattcatgtggaatataaagatggctgactttgctctttcttgactcttgttttcagtttcaattcagtgctgtacttgatgacagacacttctaaatgaagtgcaaatttgatacatatgtgaatatggactcagttttcttgcagatcaaatttcacgtcgtcttctgtatactgtggaggtacactcttatagaaagttcaaaaagtctacgctctcctttctttctaactccagtgaagtaatggggtcctgctcaagttgaaagagtcctatttgcactgtagcctcgccgtctgtgaattggaccatcctatttaactggcttcagcctccccaccttcttcagccacctctctttttcagttggctgacttccacacctagcatctcatgagtgccaagcaaaaggagagaagagagaaatagcctgcgctgttttttagtttgggggttttgctgtttccttttatgagacccattcctatttcttatagtcaatgtttcttttatcacgatattattagtaagaaaacatcactgaaatgctagctgcaagtgacatctctttgatgtcatatggaagagttaaaacaggtggagaaattccttgattcacaatgaaatgctctcctttcccctgcccccagaccttttatccacttacctagattctacatattctttaaatttcatctcaggcctccctcaaccccaccacttcttttataactagtcctttactaatccaacccatgatgagctcctcttcctggcttcttactgaaaggttaccctgtaacatgcaattttgcatttgaataaagcctgctttttaagtgttaact

SEQ ID NO: 27 - DNA, 2214 bp, Homo sapiens
  Homo sapiens solute carrier family 22 (organic cation/ergothioneine transporter), member 4 (SLC22A4)
cctgtttcccaggaacggtccccggcttcgcgccccaatttctaacagcctgcctgtcccccgggaacgttctaacatccttggggagcgccccagctacaagacactgtcctgagaacgctgtcatcacccgtagttgcaagtttcggagcggcagtgggaagcatgcgggactacgacgaggtgatcgccttcctgggcgagtgggggcccttccagcgcctcatcttcttcctgctcagcgccagcatcatccccaatggcttcaatggtatgtcagtcgtgttcctggcggggaccccggagcaccgctgtcgagtgccggacgccgcgaacctgagcagcgcctggcgcaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgagtggaatctggtgtgtgaggacaactggaaggtgcccctcaccacctccctgttcttcgtaggcgtgctcctcggctccttcgtgtccgggcagctgtcagacaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggttcattcctgaatctccccgatggctgatatcccagagaagatttagagaggctgaagatatcatccaaaaagctgcaaaaatgaacaacatagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaaccctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcctcaccctttttttccctgaaagtttgggaatgactcttccagaaaccttagagcagatgcagaaagtgaaatggttcagatctgggaaaaaaacaagagactcaatggagacagaagaaaatcccaaggttctaataactgcattctgaaaaaatatctaccccatttggtgaagtgaaaaacagaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttccaatcatacaaatactatccaaataaaaataacatcattgtattaacgcaaatattaggtgacaacaaaaaaaaaaaaaaaaaaaaaaaa

SEQ ID NO: 28 - DNA, 1593 bp, Homo sapiens
  Homo sapiens superoxide dismutase 2, mitochondrial (SOD2)
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 29 - DNA, 5455 bp, Homo sapiens
  Homo sapiens SP100 nuclear antigen (SP100)
atttgggcggagccctttctgagtcagtctgtcggccgacttcctgcttggggcctgggcagccacactgcacgcaggctgggccgactgaggggctcagaggccaggctctgaggcccacgcagggcctagggtgggaagatggcaggtgggggcggcgacctgagcaccaggaggctgaatgaatgtatttcaccagtagcaaatgagatgaaccatcttcctgcacacagccacgatttgcaaaggatgttcacggaagaccagggtgtagatgacaggctgctctatgacattgtattcaagcacttcaaaagaaataaggtggagatttcaaatgcaataaaaaagacatttccattcctcgagggcctccgtgatcgtgatctcatcacaaataaaatgtttgaagattctcaagattcttgtagaaacctggtccctgtacagagagtggtgtacaatgttcttagtgaactggagaagacatttaacctgccagttctggaagcactgttcagcgatgtcaacatgcaggaataccccgatttaattcacatttataaaggctttgaaaatgtaatccatgacaaattgcctctccaagaaagtgaagaagaagagagggaggagaggtctggcctccaactaagtcttgaacaaggaactggtgaaaactcttttcgaagcctgacttggccaccttcgggttccccatctcatgctggtacaaccccacctgaaaatggactctcagagcacccctgtgaaacagaacagataaatgcaaagagaaaagatacaaccagtgacaaagatgattcgctaggaagccaacaaacaaatgaacaatgtgctcaaaaggctgagccaacagagtcctgcgaacaaattgctgtccaagtgaataatggggatgctggaagggagatgccctgcccgttgccctgtgatgaagaaagcccagaggcagagctacacaaccatggaatccaaattaattcctgttctgtgcgactggtggatataaaaaaggaaaagccattttctaattcaaaagttgagtgccaagcccaagcaagaactcatcataaccaggcatctgacataatagtcatcagcagtgaggactctgaaggatccactgacgttgatgagcccttagaagtcttcatctcagcaccgagaagtgagcctgtgatcaataatgacaaccctttagaatcaaatgatgaaaaggagggccaagaagccacttgctcacgaccccagattgtaccagagcccatggatttcagaaaattatctacattcagagaaagttttaagaaaagagtgataggacaagaccacgacttttcagaatccagtgaggaggaggcgcccgcagaagcctcgagcggggcactgagaagcaagcatggtgagaaggctcctatgacttctagaagtacatctacttggagaatacccagcaggaagagacgtttcagcagtagtgacttttcagacctgagtaatggagaagagcttcaggaaacctgcagctcatccctaagaagagggtcaggatcacagccacaagaacctgaaaataagaagtgctcctgtgtcatgtgttttccaaaaggtgtgccaagaagccaagaagcaaggactgaaagtagtcaagcatctgacatgatggataccatggatgttgaaaacaattctactttggaaaaacacagtgggaaaagaagaaaaaagagaaggcatagatctaaagtaaatggtctccaaagagggagaaagaaagacagacctagaaaacatttaactctgaataacaaagtccaaaagaaaagatggcaacaaagaggaagaaaagccaacactagacctttgaaaagaagaagaaaaagaggtccaagaattcccaaagatgaaaatattaattttaaacaatctgaacttcctgtgacctgtggtgaggtgaagggcactctatataaggagcgattcaaacaaggaacctcaaagaagtgtatacagagtgaggataaaaagtggttcactcccagggaatttgaaattgaaggagaccgcggagcatccaagaactggaagctaagtatacgctgcggtggatataccctgaaagtcctgatggagaacaaatttctgccagaaccaccaagcacaagaaaaaagagaatactggaatctcacaacaataccttagttgacccttgtccggaaaactcaaatatatgtgaggtgtgcaacaaatggggacggctgttctgctgcgacacttgtccaagatcctttcatgagcactgccacatcccatccgtggaagctaacaagaacccgtggagttgcatcttctgcaggataaagactattcaggaaagatgcccagaaagccaatcaggtcatcaggaatctgaagtcctgatgaggcagatgctgcctgaggagcagttgaaatgtgaattcctcctcttgaaggtctactgtgattcgaaaagctgctttttcgcctcagaaccgtattataacagagaggggtctcagggcccacagaagcccatgtggttaaacaaagtcaagacaagtttgaatgagcagatgtacacccgagtagaagggtttgtgcaggacatgcgtctcatctttcataaccacaaggaattttacagggaagataaattcaccagactgggaattcaagtacaggacatctttgagaagaatttcagaaacatttttgcaattcaggaaacaagcaagaacattataatgtttatttagccattcttatctcctcccttcagatcctctggcagctagctacgcaatgtgcctgtggtcccactaatctgtgactgctcctgtggaaactccacatcacaattctccaaaatttatcattgccattttaaaaccgtcttttcagctttcaataaaattcaacaccccttcatgttaaaaattctcaataagctaggtattgaggaacatatcccaaaataataagagccatttatgacaaacccacagacaacattatatggaatgcgcaaaagaagcattccccttgaaaacaagcacaagacaaggattccctctctcaccactcctattcaacaaagtattggaagtcctggtcagagcagtcaggaagcagaaaaaaataaagggtatctaaataggcaaagaggaagtcaaactatccctgtttgcacacaacattgattctatatctagaaaaccccctagtctcagcccagaagctccttctgctgataaacaatttcagagatgtttcagaatacaaaattagtatatgaaaattactagtattcctatacaccagcaatagccaagccaagagccaaatcaggaaggcaatctcattcacaattgccactaaaagaataaaatacctaggaatacagctaatcagggaggtgagagagttctacaatgagaattacgaaacactgctcaaagagattggagatgacacaaacaaatggaaaaacatcccatgctcctgtgtagaaacagtcaatatcattaaaatgaccatactgcccaaagcagtttacaggttcaatgttattcctatcaaaccaccaatgacattcttcacagaactagataaaactattttaaaattcatacagaaccaaaaaagagcccaaatagccaaggcaatcctaagcaaaaagaacaaagctgaaggcatcacgttaccccacttcaaactatattacagggcttcagtaaccaaaacagcatggtactggtaccaaaaaaaaagccacatagaccaatggaacagaacgaagagcacagaataagaccacactcctatgaccatctgatcgtcgataaaaacaagcaatgggaaaaagactccctattttataaatggtgctgggataactgggatagaagattgaagctagacctcttccttacaccatatacaaaaatcaactcaagatcaattaaagacttaatgtaaaatcaaaaactatgaagactctggaagacaacctaggcaataccatcctggacataggaacaggcaaagatttcatgataaagacaaaagcaatagcaacaaaagcaaaatttgacaaatgggatctaattaaacttaagagattctgcacagcaaaagaaacaatcaacagagtaaacagacaacctacaaaatgggagaaaatatttgcacactatgcatctgacaaaggtctaatagccagcttctatagggaacttaaacaaatttacaagacaaaaagaaataaccccattaaaaagtgggcaaaggacatgaaagacactttttttttttaagatggagtttcactcttgttgcccaggccagagtgcaatggcgtgatcttggctcaccacaacctctgcctcccgggttcaagcaattctcctgcctcagcctcccaggtggctgggattacaggcatgcaccacctgactgattttgtattttagtagagacggggtttctccacattggtcaggctggtcttgaactcccgacctcaggtgatccacccacctcggcctcccaaagtgctgggattacaggcatcagccaccatgcccggatgaaaagacactttccaaaagaagatacacatgcggccaacaagcatgttttaaaagctcaatatcactgatcgttagagacatgcaaattaaaactacaatgagacaccatctcacaccagtcaaaatgcctctttctaaaaagtcaaaaaataacagctagtaaggttgtggagaaaagggaacatttatacactattgatgggagtgtaaattagttcaaccactgtggaaagcagtgtggcaactcctcatagtgctaaaagcagaactgccattccacccagcaatcccattactgggtacatacccagaggaatataaatcattctaccataaagacacatgcatgcaaatgtccactgcagcactattcacaatagcaaagatacagaatcaacctaagtgcccatcagtaacagattggataaagaaaatatggtacacatacaccatggaatagtatgcagccataagaaacaatgagatcatgtctcaggaacatggatagagctggaggctattatccttagcaaactaattcaggaacagaaaaccaaataccacaggttctcagttgtgagtgggagctaaatgatgagaactcatgaacacaatgaagggaacagacactagggtctacttgagggtggaggatgggaagagggagaggagcagaaaaagtacctattggtgatgaagtactctgtacaacaaacccgtgacaagagtttccctatataacaaaccttcacatatacccctgaacctaaaagtttttttaattgtaaataaatggatcattaaaaaaaattttaataat

SEQ ID NO: 30 - DNA, 5661 bp, Homo sapiens
  Homo sapiens toll-like receptor 4 (TLR4)
tagcttcctcttgctgtttctttagccactggtctgcaggcgttttcttcttctaacttcctctcctgtgacaaaagagataactattagagaaacaaaagtccagaatgctaaggttgccgctttcacttcctctcaccctttagcccagaactgctttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaactacgtgtgaaggtattcaaggcagggagtatacattgctgtttcctgttgggcaatgctccttgaccacattttgggaagagtggatgttatcattgagaaaacaatgtgtctggaattaatggggttcttataaagaaggttcccagaaaagaatgttcatccagcctcctcagaaacagaacattcaagaaaaggacaatcaggatgtcatcagggaaatgaaaataaaaaccacaatgagatatcaccttataccaggtagaatggctactataaaaaaatgaagtgtcatcaaggatatagagaaattggaacccttcttcactgctggagggaatggaaaatggtgtagccgttatgaaaaacagtacggaggtttctcaaaaattaaaaatagaactgctatatgatccagcaatctcacttctgtatatatacccaaaataattgaaatcagaatttcaagaaaatatttacactcccatgttcattgtggcactcttcacaatcactgtttccaaagttatggaaacaacccaaatttccattgaaaaataaatggacaaagaaaatgtgcatatacgtacaatgggatattattcagcctaaaaaaagggggaatcctgttatttatgacaacatgaataaacccggaggccattatgctatgtaaaatgagcaagtaacagaaagacaaatactgcctgatttcatttatatgaggttctaaaatagtcaaactcatagaagcagagaatagaacagtggttcctagggaaaaggaggaagggagaaatgaggaaatagggagttgtctaattggtataaaattatagtatgcaagatgaattagctctaaagatcagctgtatagcagagttcgtataatgaacaatactgtattatgcacttaacattttgttaagagggtacctctcatgttaagtgttcttaccatatacatatacacaaggaagcttttggaggtgatggatatatttattaccttgattgtggtgatggtttgacaggtatgtgactatgtctaaactcatcaaattgtatacattaaatatatgcagttttataatatcaattatgtctgaatgaagctataaaaaagaaaagacaacaaaattcagttgtcaaaactggaaatatgaccacagtcagaagtgtttgttactgagtgtttcagagtgtgtttggtttgagcaggtctagggtgattgaacatccctgggtgtgtttccatgtctcatgtactagtgaaagtagatgtgtgcatttgtgcacatatccctatgtatccctatcagggctgtgtgtatttgaaagtgtgtgtgtccgcatgatcatatctgtatagaagagagtgtgattatatttcttgaagaatacatccatttgaaatggatgtctatggctgtttgagatgagttctctactcttgtgcttgtacagtagtctccccttatcccttatgcttggtggatacgttcttagaccccaagtggatctctgagaccgcagatggtaccaaacctcatatatgcaatattttttcctatacataaatacctaagataaagttcatcttctgaattaggcacagtaagagattaacaataactaacaataaaattgaatagttataataatatattgtaataaaagttatgtgaatgtgatctctttctttctctctctcaaaat

SEQ ID NO: 31 - DNA, 1237 bp, Homo sapiens
  Homo sapiens chemokine (C-C motif) ligand 5 (CCL5)
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 32 - DNA, 2207 bp, Homo sapiens
  Homo sapiens chemokine (C-C motif) receptor 7 (CCR7)
cacttcctccccagacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 33 - DNA, 771 bp, Homo sapiens
  Homo sapiens CD3d molecule, delta (CD3-TCR complex) (CD3D)
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 34 - DNA, 3309 bp, Homo sapiens
  Homo sapiens CD6 molecule (CD6)
gcagaccaaaaccacaagcagaacaagcaggcgtgagacactcacaggttgggtttgatcgcatgcgtgtcggagaggagagagcagagagagacacaggaacaagaacagcaaagggtagagcagacctgcgccaggggcgcacaacggccgtgtccacctcccggccccaagatggtgcttcccacaggcagccacgcgtagcagccagagacagctccagacatgtggctcttcttcgggatcactggattgctgacggcagccctctcaggtcatccatctccagccccacctgaccagctcaacaccagcagtgcagagagtgagctctgggagccaggggagcggcttccggtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgcagagaaccgcgcgctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcacgcccggccgcgggcctatccaccgggaccaggtgaactgctcgggggccgaagcttacctgtgggactgcccggggctgccaggacagcactactgcggccacaaagaggacgcgggcgcggtgtgctcagagcaccagtcctggcgcctgacagggggcgctgaccgctgcgaggggcaggtggaggtacacttccgaggggtctggaacacagtgtgtgacagtgagtggtacccatcggaggccaaggtgctctgccagtccttgggctgtggaactgcggttgagaggcccaaggggctgccccactccttgtccggcaggatgtactactcatgcaatggggaggagctcaccctctccaactgctcctggcggttcaacaactccaacctctgcagccagtcgctggcagccagggtcctctgctcagcttcccggagtttgcacaatctgtccactcccgaagtccctgcaagtgttcagacagtcactatagaatcttctgtgacagtgaaaatagagaacaaggaatctcgggagctaatgctcctcatcccctccatcgttctgggaattctcctccttggctccctcatcttcatagccttcatcctcttgagaattaaaggaaaatatgccctccccgtaatggtgaaccaccagcacctacccaccaccatcccggcagggagcaatagctatcaaccggtccccatcaccatccccaaagaagttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcctaggccggggccagccgaggctcctggggtggctctgaccctctggcctcctgctctacctactccctttcccctttcccaccctcccagctcacctccccatggagctgagaggcctcccttggagagatggaaggaaacgttataccttgtacccctcggtctccatccatcaagccaaacctgctgccacagccctcccccggccccagatagcagccccagggaggatgctgcctccaagaggtgtgagccctctgtctcggggatgaacaagcagagtctgggctacctcttgacagctggtggaggggagttggggagctggactggatgactctggaggccccttccaaacctcaagtgtccggcgctttgattgcctgagtttctgacacttcagggcccagaggtcctgcgaggggcagaactggacccccatgccagtgctgctgcaggagggcccatatactagggtctgctgagctgttgtcactgatcggtgggcgctgggggggtagggtagcacaccagctgtcccaggctttgctccgggcggtaactgcacttgggcagggaatatagccttcctgggcacaactagctgacaatgacaggttgactgtgtacccccaaccaaggagctggggcccaaggccagtcctgccccagagacactccaagtccgccaggggcacagaccagttctgcagtgactgtccctggacaatgggtctttattctgagtttcctatggtttacaaagagggccccagcccagccccaccacagatcccagagataggggcccagtctccatgggggcaaggagcatagagatgttttccaggaaggggctcagaagctgcactaggccccgagtccccatgtgtctccttgaattgatgaggatgctcctgggagggatgcgtgactatgtggtgttgcacccggggctgcaaacgtctccgtgcagcccccagagagaggcccatgggctcagaccaggctttgttgtcctgctctgagtatcctgagattaaactgaattgctgaatgaaaaaaaaaaaaaaaaaaaaa

SEQ ID NO: 35 - DNA, 3109 bp, Homo sapiens
  Homo sapiens Fas apoptotic inhibitory molecule 3 (FAIM3)
agcctgagaatagttagcaaacaagggaggttgtcatttcctcatcgtcaagctttgttcctcgtgggggctagaaatctctttccagttccagattgtgaagggttcctgagtaagcagcgtgtctccatccccctctctaggggctcttggatggaccttgcactctagaagggacaatggacttctggctttggccactttacttcctgccagtatcgggggccctgaggatcctcccagaagtaaaggtagagggggagctgggcggatcagttaccatcaagtgcccacttcctgaaatgcatgtgaggatatatctgtgccgggagatggctggatctggaacatgtggtaccgtggtatccaccaccaacttcatcaaggcagaatacaagggccgagttactctgaagcaatacccacgcaagaatctgttcctagtggaggtaacacagctgacagaaagtgacagcggagtctatgcctgcggagcgggcatgaacacagaccggggaaagacccagaaagtcaccctgaatgtccacagtgaatacgagccatcatgggaagagcagccaatgcctgagactccaaaatggtttcatctgccctatttgttccagatgcctgcatatgccagttcttccaaattcgtaaccagagttaccacaccagctcaaaggggcaaggtccctccagttcaccactcctcccccaccacccaaatcacccaccgccctcgagtgtccagagcatcttcagtagcaggtgacaagccccgaaccttcctgccatccactacagcctcaaaaatctcagctctggaggggctgctcaagccccagacgcccagctacaaccaccacaccaggctgcacaggcagagagcactggactatggctcacagtctgggagggaaggccaaggatttcacatcctgatcccgaccatcctgggccttttcctgctggcacttctggggctggtggtgaaaagggccgttgaaaggaggaaagccctctccaggcgggcccgccgactggccgtgaggatgcgcgccctggagagctcccagaggccccgcgggtcgccgcgaccgcgctcccaaaacaacatctacagcgcctgcccgcggcgcgctcgtggagcggacgctgcaggcacaggggaggcccccgttcccggccccggagcgccgttgccccccgccccgctgcaggtgtctgaatctccctggctccatgccccatctctgaagaccagctgtgaatacgtgagcctctaccaccagcctgccgccatgatggaggacagtgattcagatgactacatcaatgttcctgcctgacaactccccagctatcccccaaccccaggctcggactgtggtgccaaggagtctcatctatctgctgatgtccaatacctgcttcatgtgttctcagagccctcatcacttcccatgccccatctcgactcccatccccatctatctgtgccctgagcatggctctgcccccaggtcgtcttgcacaccttggcagccccctgtagttgacaggtaagctgtaggcatgtagagcaattgtcccaatgccacttgcttcctttccaagccgtcgaacagactgtgggatttgcagagtgtttcttccatgtctttgaccacagggttgttgctgcccaggctctagatcacatggcatcaggctggggcagaggcatagctattgtctcgggcatccttcccagggttgggtcttacacaaatagaaggctcttgctctgagttatgtgacatgcctcagccccatggactaagcaggggtctggtataaaaacactcctggaaacgcctttgccctgatccaaatgttagcacttgctagtgaacgtctacttatctcaagttctatgctaaaggcaatttatcttgatgtgatgataaaccaaacttattagcaagatatgcatatatatccataaattctctttactctgtctccatcacttgatgcacataagtgccctgacctcagcatctcccctctaaaaaaaaaaaaaaaaaagtatctctttatctttcttccatagcctgacactgatatttgtgcacttaccttaactttggtctattttattcatccaaaaccattacatttcttggttttcacaaatgttccccatttcttagccagttccagacaatgtatagcaagcaggggaaggaaagcagtcaggagttcctgggtggccacggctctgcaatagcacttatgtcatggaagtgatatcccacctcctacatatactctttgcctaggtttttggaacaaggttatagtcagacactgtatctttagattgatgtcgaccacaaagttcagccagagcttgaggctagatgcacagccttgctattgggaagaaggccttttctagctgtacaacacagtctcactgggcattcatccagaaatagagaagaaagtctgccagacttgagttatgttgtcttttattagcagggaatgtcatcacagattggatagtacatccaggtgcaatgtcaccatcagcaaggtcagcttgacactcaagtggaagattagggaagaatgactaggataaaaaaaaaaggagggcaccaagggaaagggatgatggggtgagctggcgagtgtgggtgggaaatgaaatgtttattgaggatctgctttgtgctgggcactttaatccacattttatcgtttacttttcaaacagatgcaccttacccccaccccaatgctctgtccctgcagatatcagaagacagtgtgattttcatgctctgaagttcagttttacatccaagcatccctctctgttttttaacaatccaaagacaggccaaaaaaagcaccacagtttattaagtacttactaagcacccatccactgccccacactgtggcaaggattgtgaggggtaaagaagcatggggcacaatattctgctgccttcatgtaacttacagtctcacaaataaatagaacttcagttgaaatactgacattaattaaatagagttgtaataaaaaaaaaaaaaaaaaaaaaaaaaa

SEQ ID NO: 36 - DNA, 1198 bp, Homo sapiens
  Homo sapiens Fc fragment of IgE, high affinity I, receptor for; alpha polypeptide (FCER1A)
tactaagagtctccagcatcctccacctgtctaccaccgagcatgggcctatatttgaagccttagatctctccagcacagtaagcaccaggagtccatgaagaagatggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgcaatatacatagaaacgtctgtgctcaaggatttatagaaatgcttcattaaactgagtgaaactggttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtaaaaaaaaaaaaaaaaaaaaaaaaaaa

SEQ ID NO: 37 - DNA, 1074 bp, Homo sapiens
  Homo sapiens granzyme K (granzyme 3; tryptase II) (GZMK)
gatcaacacatttcatctgggcttcttaaatctaaatctttaaaatgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgtaagtcattttattaaggaatcaagttctttttcacttgtatcactgatgtatttctaccatgctggttttattctaaataaaatttagaagactcaaaaaaaaaaaaaaaaaaaaaaaaaaaaa

SEQ ID NO: 38 - DNA, 4617 bp, Homo sapiens
  Homo sapiens interleukin 7 receptor (IL7R)
atctaagcttctctgtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaagaaagaaaggaaaataaaaaatgatagttgccattattaggatttaatatatatccagtgctttgcaagtgctctgcgcaccttgtctcactccatcctgacaataatcctgggaggtgtgtgcaattactacgactactctcttttttatagatcattaaattcagaactaaggagttaagtaacttgtccaagttgttcacacagtgaagggaggggccaagatatgatggctgggagtctaattgcagttccctgagccatgtgcctttctcttcactgaggactgccccattcttgagtgccaaacgtcactagtaacagggtgtgcctagataatttatgatccaaactgagtcagtttggaaagtgaaagggaaacttacatataatccctccgggacaatgagcaaaaactaggactgtccccagacaaatgtgaacatacatatcatcacttaaattaaaatggctatgagaaagaaagagggggagaaacagtcttgcgggtgtgaagtcccatgaccagccatgtcaaaagaaggtaaagaagtcaagaaaaagccatgaagcccatttggtttcatttttctgaaaataggctcaagagggaataaattagaaactcacaatttctcttgtttgttaccaagacagtgattctcttgctgctaccacccaactgcatccgtccatgatctcagaggaaactgtcgctgaccctggacatgggtacgtttgacgagtgagaggaggcatgacccctcccatgtgtatagacactaccccaacctaaattcatccctaaattgtcccaagttctccagcaatagaggctgccacaaacttcagggagaaagagttacaagtacatgcaatgagtgaactgactgtggctacaatcttgaagatatacggaagagacgtattattaatgcttgacatatatcatcttgcctttcttggtctagactgacttctaatgactaactcaaagtcaaggcaactgagtaatgtcagctcagcaaagtgcagcaaacccatctcccacaggcctccaaaccctggctgttcacagaaccacaaagggcagatgctgcacagaaaactagagaaggggtcataggttcatggttttgtttgagatttgttgctactgtttttctgttttgaattttcttctttgttctgtttttactttatttagggggactaggtgtttctgatattttagttttcttgtttgttttgttttgtgttgtctgtgaatggggttttaactgtggatgaatggaccttatctgttggcttaaaggactggtaagatcagaccatcttattcttcaggtgaatgttttactttccaaagtgctctcctctgcaccagcagtaataaatacaatgccataatcccttaggtttgcctagtgcttttgcaattttcaaagcacttccataagcattccttccacctccttgataggcatttatggaaagcctgctacatgtcaatcatactgttaggcacaggggacctaaagacacataaaaggatggcattctgcctcataaattgcaaaacctaatgaaagtgactgcttggtaaacaaattattattatattataaaatgctataaaagagccatattgaaagtgccctgttggagacagggcaaatgccacaaaaatgatgtaaatttacatggaggaaaagtagaatctgcctggtttgtaggcagcagaagacatttttcatcagtgggcaggtgttctttaccttttgtagaaatgggagtcaagtctcaaataggaggctccacaaaatctcatgccaggtctctgataccttattcacagaagttctttgaagtatttattgttattttctttgacttatgggaaaactgggacacaggaagacaggtaaattacccaacctcacacgttaagtcagaactgggagccataattttgtatccctggtataaatagacaatctcttgaagaaatgaagagatgaccatagaaaaacatcgagatatctccagctctaaaatcctttgtttcaatgttgtttggcatatgttatctttggaatttagtgtctgagcctctgtctgttactgtagtatttaaaatgcatgtattataatcatataatcataactgctgttaattcttgattatatacctagggacaatgtgtaatgtaagattactaattggttctgcccaatctcctttcagattttattaggaaaaaaaaataaacctcctgatcggagacaatgtattaatcagaagtgtaaactgccagttctatatagcatgaaatgaaaagacagctaatttggtccaacaaacatgactgggtctagggcacccaggctgattcagctgatttcctaccagcctttgcctcttccttcaatgtggtttccatgggaatttgcttcagaaaagccaagtatgggctgttcagaggtgcacacctgcattttcttagctcttctagaggggctaagagacttggtacgggccaggaagaatatgtggcagagctcctggaaatgatgcagattaggtggcatttttgtcagctctgtggtttattgttgggactattctttaaaatatccattgttcactacagtgaagatctctgatttaaccgtgtactatccacatgcattacaaacatttcgcagagctgcttagtatataagcgtacaatgtatgtaataaccatctcatatttaattaaatggtatagaagaacaaaaaaaaaaaaaaaaaa

SEQ ID NO: 39 - DNA, 740 bp, Homo sapiens
  Homo sapiens killer cell lectin-like receptor subfamily B, member 1 (KLRB1)
gcctcacagaattgagagtttgttcttacacacaagtttaatgccaccttcctctgtctgccatggaccaacaagcaatatatgctgagttaaacttacccacagactcaggcccagaaagttcttcaccttcatctcttcctcgggatgtctgtcagggttcaccttggcatcaatttgccctgaaacttagctgtgctgggattattctccttgtcttggttgttactgggttgagtgtttcagtgacatccttaatacagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtggataaacggctcttttttaaattctaatgacttagaaattagaggtgatgctaaagaaaacagctgtatttccatctcacagacatctgtgtattctgagtactgtagtacagaaatcagatggatctgccaaaaagaactaacacctgtgagaaataaagtgtatcctgactcttga

SEQ ID NO: 40 - DNA, 859 bp, Homo sapiens
  Homo sapiens mal, T-cell differentiation protein (MAL)
tcttctgccccgggctcccctgctcttaacccgcgcgcgggggcgcccaggccactgggctccgcggagccagcgagaggtctgcgcggagtctgagcggcgctcgtcccgtcccaaggccgacgccagcacgccgtcatggcccccgcagcggcgacggggggcagcaccctgcccagtggcttctcggtcttcaccaccttgcccgacttgctcttcatctttgagtttgtgttctcctacatagccactctgctctacgtggtccatgcggtgttctctttaatcagatggaagtcttcataaagccgcagtagaacttgagctgaaaacccagatggtgttaactggccgccccactttccggcataactttttagaaaacagaaatgcccttgatggtggaaaaaagaaaacaaccacccccccactgcccaaaaaaaaaagccctgccctgttgctcgtgggtgctgtgtttactctcccgtgtgccttcgcgtccgggttgggagcttgctgtgtctaacctccaactgctgtgctgtctgctagggtcacctcctgtttgtgaaaggggaccttcttgttcgggggtgggaagtggcgaccgtgacctgagaaggaaagaaagatcctctgctgacccctggagcagctctcgagaactacctgttggtattgtccacaagctctcccgagcgccccatcttgtgccatgttttaagtcttcatggatgttctgcatgtcatggggactaaaactcacccaacagatctttccagaggtccatggtggaagacgataaccctgtgaaatactttataaaatgtcttaatgttcaaaaaaaaaaaaaaaaaa

SEQ ID NO: 41 - DNA, 1435 bp, Homo sapiens
  Homo sapiens hypoxanthine phosphoribosyltransferase 1 (HPRT1)
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 42 - DNA, 1421 bp, Homo sapiens
  Homo sapiens glyceraldehyde-3-phosphate dehydrogenase (GAPDH)
gcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 43 - protein, 4 aa, Homo sapiens
DEAD

SEQ ID NO: 44 - DNA, 20 bp, Artificial Sequence
  Description of Artificial Sequence Synthetic primer
gctctcggaaaccagaccaa

SEQ ID NO: 45 - DNA, 20 bp, Artificial Sequence
  Description of Artificial Sequence Synthetic primer
gttggacaccgaggaacagt

SEQ ID NO: 46 - DNA, 20 bp, Artificial Sequence
  Description of Artificial Sequence Synthetic primer
aactccgtacaagcatgcga

SEQ ID NO: 47 - DNA, 20 bp, Artificial Sequence
  Description of Artificial Sequence Synthetic primer
ccacggccaatacatcgtct

SEQ ID NO: 48 - DNA, 20 bp, Artificial Sequence
  Description of Artificial Sequence Synthetic primer
accctacccacctcctcaag

SEQ ID NO: 49 - DNA, 20 bp, Artificial Sequence
  Description of Artificial Sequence Synthetic primer
ctgaggactgcacgtatgct

SEQ ID NO: 50 - DNA, 20 bp, Artificial Sequence
  Description of Artificial Sequence Synthetic primer
ttgaggtgtcatgcgtggaa

SEQ ID NO: 51 - DNA, 20 bp, Artificial Sequence
  Description of Artificial Sequence Synthetic primer
ggagtgattgcagctctggt

SEQ ID NO: 52 - DNA, 20 bp, Artificial Sequence
  Description of Artificial Sequence Synthetic primer
ggctacactgttctgcggat

SEQ ID NO: 53 - DNA, 20 bp, Artificial Sequence
  Description of Artificial Sequence Synthetic primer
cctgtttgagggcatcgact

SEQ ID NO: 54 - DNA, 20 bp, Artificial Sequence
  Description of Artificial Sequence Synthetic primer
caacatccacagcgagacct

SEQ ID NO: 55 - DNA, 20 bp, Artificial Sequence
  Description of Artificial Sequence Synthetic primer
catgtcgtctggtccctgtt

SEQ ID NO: 56 - DNA, 20 bp, Artificial Sequence
  Description of Artificial Sequence Synthetic primer
accactacagcaagggcttc

SEQ ID NO: 57 - DNA, 20 bp, Artificial Sequence
  Description of Artificial Sequence Synthetic primer
ccagcaagatgcaagccttc

SEQ ID NO: 58 - DNA, 20 bp, Artificial Sequence
  Description of Artificial Sequence Synthetic primer
gattccggtctcaggagcag

SEQ ID NO: 59 - DNA, 20 bp, Artificial Sequence
  Description of Artificial Sequence Synthetic primer
cagacagcgaccaaaaagcc

SEQ ID NO: 60 - DNA, 20 bp, Artificial Sequence
  Description of Artificial Sequence Synthetic primer
aactggggcaggattgtgac

SEQ ID NO: 61 - DNA, 21 bp, Artificial Sequence
  Description of Artificial Sequence Synthetic primer
cctcacgagactccccataga

SEQ ID NO: 62 - DNA, 20 bp, Artificial Sequence
  Description of Artificial Sequence Synthetic primer
aggccacgcaaaaactttcc

SEQ ID NO: 63 - DNA, 20 bp, Artificial Sequence
  Description of Artificial Sequence Synthetic primer
acaacatagcatccccgtgt

SEQ ID NO: 64 - DNA, 20 bp, Artificial Sequence
  Description of Artificial Sequence Synthetic primer
agtgcagaggagatgcccta

SEQ ID NO: 65 - DNA, 20 bp, Artificial Sequence
  Description of Artificial Sequence Synthetic primer
cgccacagcctccattaaac

SEQ ID NO: 66 - DNA, 20 bp, Artificial Sequence
  Description of Artificial Sequence Synthetic primer
aggactcccaatgtggtgga

SEQ ID NO: 67 - DNA, 20 bp, Artificial Sequence
  Description of Artificial Sequence Synthetic primer
tgccatcgtctgctatgacc

SEQ ID NO: 68 - DNA, 20 bp, Artificial Sequence
  Description of Artificial Sequence Synthetic primer
cggaaggggcattttgacac

SEQ ID NO: 69 - DNA, 20 bp, Artificial Sequence
  Description of Artificial Sequence Synthetic primer
gaactggggagatggtgagc

SEQ ID NO: 70 - DNA, 20 bp, Artificial Sequence
  Description of Artificial Sequence Synthetic primer
gttcagccaggacgtctacc

SEQ ID NO: 71 - DNA, 20 bp, Artificial Sequence
  Description of Artificial Sequence Synthetic primer
aaacctcagccctaacggtg

SEQ ID NO: 72 - DNA, 20 bp, Artificial Sequence
  Description of Artificial Sequence Synthetic primer
cttgctcacgaccccagatt

SEQ ID NO: 73 - DNA, 20 bp, Artificial Sequence
  Description of Artificial Sequence Synthetic primer
cattggtgtgtcggtcctca

SEQ ID NO: 74 - DNA, 20 bp, Artificial Sequence
  Description of Artificial Sequence Synthetic primer
cttgcccgacttgctcttca

SEQ ID NO: 75 - DNA, 20 bp, Artificial Sequence
  Description of Artificial Sequence Synthetic primer
cttgtcatcatccgcaccct

SEQ ID NO: 76 - DNA, 20 bp, Artificial Sequence
  Description of Artificial Sequence Synthetic primer
gttactacaacggcgaccct

SEQ ID NO: 77 - DNA, 20 bp, Artificial Sequence
  Description of Artificial Sequence Synthetic primer
ccagatggcgtgttagcagt

SEQ ID NO: 78 - DNA, 20 bp, Artificial Sequence
  Description of Artificial Sequence Synthetic primer
gagccatcatgggaagagca

SEQ ID NO: 79 - DNA, 20 bp, Artificial Sequence
  Description of Artificial Sequence Synthetic primer
gtctatcagcccctccgaga

SEQ ID NO: 80 - DNA, 20 bp, Artificial Sequence
  Description of Artificial Sequence Synthetic primer
atgaggaggtccagcaaagc

SEQ ID NO: 81 - DNA, 21 bp, Artificial Sequence
  Description of Artificial Sequence Synthetic primer
tgaaacttagctgtgctggga

SEQ ID NO: 82 - DNA, 20 bp, Artificial Sequence
  Description of Artificial Sequence Synthetic primer
ccaaccggcagcaatgtatg

SEQ ID NO: 83 - DNA, 20 bp, Artificial Sequence
  Description of Artificial Sequence Synthetic primer
cagtcgtctttgtcacccga

SEQ ID NO: 84 - DNA, 20 bp, Artificial Sequence
  Description of Artificial Sequence Synthetic primer
cctggcgtcgtgattagtga

SEQ ID NO: 85 - DNA, 20 bp, Artificial Sequence
  Description of Artificial Sequence Synthetic primer
cctggcgtcgtgattagtga

SEQ ID NO: 86 - DNA, 20 bp, Artificial Sequence
  Description of Artificial Sequence Synthetic primer
aagcccttctggatcagtgc

SEQ ID NO: 87 - DNA, 20 bp, Artificial Sequence
  Description of Artificial Sequence Synthetic primer
cgctgtgcatttgacctctc

SEQ ID NO: 88 - DNA, 20 bp, Artificial Sequence
  Description of Artificial Sequence Synthetic primer
ggcattttctgcagcacctc

SEQ ID NO: 89 - DNA, 20 bp, Artificial Sequence
  Description of Artificial Sequence Synthetic primer
ttggtcacgttgagggatgg

SEQ ID NO: 90 - DNA, 20 bp, Artificial Sequence
  Description of Artificial Sequence Synthetic primer
aggtggatggtcctagctca

SEQ ID NO: 91 - DNA, 20 bp, Artificial Sequence
  Description of Artificial Sequence Synthetic primer
tgtaaatcgcactcgcggta

SEQ ID NO: 92 - DNA, 20 bp, Artificial Sequence
  Description of Artificial Sequence Synthetic primer
tgcctgagcaatggtaggtg

SEQ ID NO: 93 - DNA, 20 bp, Artificial Sequence
  Description of Artificial Sequence Synthetic primer
gacctgctcagtcgtgttca

SEQ ID NO: 94 - DNA, 20 bp, Artificial Sequence
  Description of Artificial Sequence Synthetic primer
cacccagatcacaagcccat

SEQ ID NO: 95 - DNA, 20 bp, Artificial Sequence
  Description of Artificial Sequence Synthetic primer
tcgtgaatctgggccttgtc

SEQ ID NO: 96 - DNA, 20 bp, Artificial Sequence
  Description of Artificial Sequence Synthetic primer
tcgccaaccatcttcctgtc

SEQ ID NO: 97 - DNA, 20 bp, Artificial Sequence
  Description of Artificial Sequence Synthetic primer
gtcgccaaccatcttcctgt

SEQ ID NO: 98 - DNA, 20 bp, Artificial Sequence
  Description of Artificial Sequence Synthetic primer
atcgtgcacataagcctcgt

SEQ ID NO: 99 - DNA, 20 bp, Artificial Sequence
  Description of Artificial Sequence Synthetic primer
gacttgacacaggacaggca

SEQ ID NO: 100 - DNA, 20 bp, Artificial Sequence
  Description of Artificial Sequence Synthetic primer
gaatcccaaggaccaccagg

SEQ ID NO: 101 - DNA, 20 bp, Artificial Sequence
  Description of Artificial Sequence Synthetic primer
atttcggcaggtggttgaca

SEQ ID NO: 102 - DNA, 20 bp, Artificial Sequence
  Description of Artificial Sequence Synthetic primer
cccatcccagcctctttgtt

SEQ ID NO: 103 - DNA, 20 bp, Artificial Sequence
  Description of Artificial Sequence Synthetic primer
cgcaagtctagcctcctctt

SEQ ID NO: 104 - DNA, 20 bp, Artificial Sequence
  Description of Artificial Sequence Synthetic primer
tgatgccagtgctccgattt

SEQ ID NO: 105 - DNA, 20 bp, Artificial Sequence
  Description of Artificial Sequence Synthetic primer
tgagcaccccagtttcatca

SEQ ID NO: 106 - DNA, 20 bp, Artificial Sequence
  Description of Artificial Sequence Synthetic primer
attccacacggcagccataa

SEQ ID NO: 107 - DNA, 20 bp, Artificial Sequence
  Description of Artificial Sequence Synthetic primer
tccgctgcaaagtaaaccct

SEQ ID NO: 108 - DNA, 20 bp, Artificial Sequence
  Description of Artificial Sequence Synthetic primer
tcccagtttgcccataggtg

SEQ ID NO: 109 - DNA, 20 bp, Artificial Sequence
  Description of Artificial Sequence Synthetic primer
cgcagttccttcacccagaa

SEQ ID NO: 110 - DNA, 21 bp, Artificial Sequence
  Description of Artificial Sequence Synthetic primer
tggtgtttgcaagctcctttg

SEQ ID NO: 111 - DNA, 20 bp, Artificial Sequence
  Description of Artificial Sequence Synthetic primer
tagtttgtggcaggcgtcat

SEQ ID NO: 112 - DNA, 20 bp, Artificial Sequence
  Description of Artificial Sequence Synthetic primer
gcaccttccagttgtcctca

SEQ ID NO: 113 - DNA, 20 bp, Artificial Sequence
  Description of Artificial Sequence Synthetic primer
gaaaccaagccaaccccaac

SEQ ID NO: 114 - DNA, 20 bp, Artificial Sequence
  Description of Artificial Sequence Synthetic primer
ggagccttctcaccatgctt

SEQ ID NO: 115 - DNA, 20 bp, Artificial Sequence
  Description of Artificial Sequence Synthetic primer
ccagtcctcatcctggcttg

SEQ ID NO: 116 - DNA, 19 bp, Artificial Sequence
  Description of Artificial Sequence Synthetic primer
agaacaccgcatggaccac

SEQ ID NO: 117 - DNA, 20 bp, Artificial Sequence
  Description of Artificial Sequence Synthetic primer
gagctcacaggtgctactgg

SEQ ID NO: 118 - DNA, 20 bp, Artificial Sequence
  Description of Artificial Sequence Synthetic primer
agattccaggctttgtggca

SEQ ID NO: 119 - DNA, 20 bp, Artificial Sequence
  Description of Artificial Sequence Synthetic primer
tgaaaggctgccattgtgga

SEQ ID NO: 120 - DNA, 20 bp, Artificial Sequence
  Description of Artificial Sequence Synthetic primer
gagtggtgaactggagggac

SEQ ID NO: 121 - DNA, 20 bp, Artificial Sequence
  Description of Artificial Sequence Synthetic primer
acttgttccgagcccagttt

SEQ ID NO: 122 - DNA, 20 bp, Artificial Sequence
  Description of Artificial Sequence Synthetic primer
aggtgctcgactcactgttg

SEQ ID NO: 123 - DNA, 20 bp, Artificial Sequence
  Description of Artificial Sequence Synthetic primer
ctctcggagttgctgccaat

SEQ ID NO: 124 - DNA, 20 bp, Artificial Sequence
  Description of Artificial Sequence Synthetic primer
aggatccatctcccctgagc

SEQ ID NO: 125 - DNA, 21 bp, Artificial Sequence
  Description of Artificial Sequence Synthetic primer
gttgatgtactcccgaaccca

SEQ ID NO: 126 - DNA, 20 bp, Artificial Sequence
  Description of Artificial Sequence Synthetic primer
cgagcaagacgttcagtcct

SEQ ID NO: 127 - DNA, 20 bp, Artificial Sequence
  Description of Artificial Sequence Synthetic primer
ctcgctcctggaagatggtg

SEQ ID NO: 128 - DNA, 20 bp, Artificial Sequence
  Description of Artificial Sequence Synthetic primer
accctacccacctcctcaag

SEQ ID NO: 129 - DNA, 20 bp, Artificial Sequence
  Description of Artificial Sequence Synthetic primer
caacatccacagcgagacct

SEQ ID NO: 130 - DNA, 20 bp, Artificial Sequence
  Description of Artificial Sequence Synthetic primer
ggagtgattgcagctctggt

SEQ ID NO: 131 - DNA, 20 bp, Artificial Sequence
  Description of Artificial Sequence Synthetic primer
cttgctcacgaccccagatt

SEQ ID NO: 132 - DNA, 20 bp, Artificial Sequence
  Description of Artificial Sequence Synthetic primer
catgtcgtctggtccctgtt

SEQ ID NO: 133 - DNA, 20 bp, Artificial Sequence
  Description of Artificial Sequence Synthetic primer
aaacctcagccctaacggtg

SEQ ID NO: 134 - DNA, 20 bp, Artificial Sequence
  Description of Artificial Sequence Synthetic primer
ccacggccaatacatcgtct

SEQ ID NO: 135 - DNA, 20 bp, Artificial Sequence
  Description of Artificial Sequence Synthetic primer
aggactcccaatgtggtgga

SEQ ID NO: 136 - DNA, 25 bp, Artificial Sequence
  Description of Artificial Sequence Synthetic primer
tcaggcagtataatccaaagatggt

SEQ ID NO: 137 - DNA, 20 bp, Artificial Sequence
  Description of Artificial Sequence Synthetic primer
cctgtttgagggcatcgact

SEQ ID NO: 138 - DNA, 20 bp, Artificial Sequence
  Description of Artificial Sequence Synthetic primer
cggaaggggcattttgacac

SEQ ID NO: 139 - DNA, 20 bp, Artificial Sequence
  Description of Artificial Sequence Synthetic primer
aactggggcaggattgtgac

SEQ ID NO: 140 - DNA, 20 bp, Artificial Sequence
  Description of Artificial Sequence Synthetic primer
aggtggatggtcctagctca

SEQ ID NO: 141 - DNA, 20 bp, Artificial Sequence
  Description of Artificial Sequence Synthetic primer
tcgccaaccatcttcctgtc

SEQ ID NO: 142 - DNA, 20 bp, Artificial Sequence
  Description of Artificial Sequence Synthetic primer
gacctgctcagtcgtgttca

SEQ ID NO: 143 - DNA, 20 bp, Artificial Sequence
  Description of Artificial Sequence Synthetic primer
ggagccttctcaccatgctt

SEQ ID NO: 144 - DNA, 20 bp, Artificial Sequence
  Description of Artificial Sequence Synthetic primer
gtcgccaaccatcttcctgt

SEQ ID NO: 145 - DNA, 20 bp, Artificial Sequence
  Description of Artificial Sequence Synthetic primer
gaaaccaagccaaccccaac

SEQ ID NO: 146 - DNA, 20 bp, Artificial Sequence
  Description of Artificial Sequence Synthetic primer
ttggtcacgttgagggatgg

SEQ ID NO: 147 - DNA, 20 bp, Artificial Sequence
  Description of Artificial Sequence Synthetic primer
tcccagtttgcccataggtg

SEQ ID NO: 148 - DNA, 25 bp, Artificial Sequence
  Description of Artificial Sequence Synthetic primer
agtctggcttatatccaacacttcg

SEQ ID NO: 149 - DNA, 20 bp, Artificial Sequence
  Description of Artificial Sequence Synthetic primer
tcgtgaatctgggccttgtc

SEQ ID NO: 150 - DNA, 21 bp, Artificial Sequence
  Description of Artificial Sequence Synthetic primer
tggtgtttgcaagctcctttg

SEQ ID NO: 151 - DNA, 20 bp, Artificial Sequence
  Description of Artificial Sequence Synthetic primer
cccatcccagcctctttgtt

SEQ ID NO: 152 - DNA, 20 bp, Artificial Sequence
  Description of Artificial Sequence Synthetic probe
tacggctggcagggtggacc

SEQ ID NO: 153 - DNA, 27 bp, Artificial Sequence
  Description of Artificial Sequence Synthetic probe
ccgtgcccgaccatgtcgtctggtccc

SEQ ID NO: 154 - DNA, 27 bp, Artificial Sequence
  Description of Artificial Sequence Synthetic probe
tgtcctttggtcactgcaccatcgtga

SEQ ID NO: 155 - DNA, 20 bp, Artificial Sequence
  Description of Artificial Sequence Synthetic probe
agtgaggaggaggcgcccgc

SEQ ID NO: 156 - DNA, 21 bp, Artificial Sequence
  Description of Artificial Sequence Synthetic probe
acccctgctgcctgggcttca

SEQ ID NO: 157 - DNA, 27 bp, Artificial Sequence
  Description of Artificial Sequence Synthetic probe
acggctgcatctgttggtgtccaaggc

SEQ ID NO: 158 - DNA, 25 bp, Artificial Sequence
  Description of Artificial Sequence Synthetic probe
gctcagtgaacatccagatggcccc

SEQ ID NO: 159 - DNA, 25 bp, Artificial Sequence
  Description of Artificial Sequence Synthetic probe
tgtgctgtgctgtcagtatctgggt

SEQ ID NO: 160 - DNA, 25 bp, Artificial Sequence
  Description of Artificial Sequence Synthetic probe
caagcttgctggtgaaaaggacccc

SEQ ID NO: 161 - DNA, 20 bp, Artificial Sequence
  Description of Artificial Sequence Synthetic probe
agcaccctggagcccgtgga

SEQ ID NO: 162 - DNA, 26 bp, Artificial Sequence
  Description of Artificial Sequence Synthetic probe
agggtgagctgaagcagctgcttaca

SEQ ID NO: 163 - DNA, 25 bp, Artificial Sequence
  Description of Artificial Sequence Synthetic probe
tcgtaaggacaaaacgggactggct

(19) United States
(12) Patent Application Publication
(10) Pub. No.: US 2016/0244834 A1
(43) Pub. Date: Aug. 25, 2016

(54) SEPSIS BIOMARKERS AND USES THEREOF

(71) Applicant: ACUMEN RESEARCH LABORATORIES PTE. LTD., Singapore (SG)

(72) Inventors: Siew H. Ong, Singapore (SG); Win S. Kuan, Singapore (SG); Di Wu, Singapore (SG)

(21) Appl. No.: 14/900,416

(22) PCT Filed: Jun. 27, 2014
(86) PCT No.: PCT/SG2014/000312

§ 371 Date: Dec. 21, 2015

(30) Foreign Application Priority Data
Jun. 28, 2013 (SG) 201305082-8

(51) Int. Cl. C12Q 1/68

(52) U.S. Cl. CPC C12Q 1/6883; C12Q 2600/118; C12Q 2600/158

(57) ABSTRACT

Worldwide incidence rate of sepsis continues to rise, with increasing concern in the elderly patients due to fast aging population. There is a need for effective biomarkers for diagnosis and/or prognosis of sepsis. The present invention relates to diagnostic and/or prognostic biomarker or biomarkers for detection and/or prediction of sepsis. The present invention discloses a predetermined panel of genes which are biomarkers for detection and/or prognosis of sepsis in a subject, including the states or conditions in the sepsis continuum.

### FIELD OF THE INVENTION

The present invention relates to diagnostic and/or prognostic biomarker or biomarkers for detection and/or prediction of sepsis.

### BACKGROUND OF THE INVENTION

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

Sepsis arises from a host response to an infection caused by bacteria or other infectious agents such as viruses, fungi and parasites. This response is called Systemic Inflammatory Response Syndrome (SIRS). Outcomes from sepsis are determined by the virulence of the invading pathogen and the host response, which may be over-exuberant resulting in collateral damage of organs and tissues. Typically, when sepsis arises, the body of the host is unable to break down clots that are formed in the lining of inflamed blood vessels, limiting blood flow to the organs, and subsequently leading to organ failure or gangrene.

Sepsis is a continuum of heterogeneous disease processes generally starting with infection, followed by SIRS, then sepsis, followed by severe sepsis and finally septic shock which causes multiple organ dysfunction and death. Worldwide incidence of sepsis continues to rise, with increasing concern in the elderly patients due to fast aging population. Approximately one-third to one-half of all severe sepsis patients succumb to their illness. Early stratification and timely intervention in patients with suspected infection before progression to sepsis remains a critical clinical challenge to physicians worldwide as sepsis is often diagnosed at too late a stage.

Early diagnosis of sepsis is challenging because clinical signs of SIRS in sepsis are antedated by biochemical and immunological reactions. In addition, SIRS criteria are very generic in which border line outcomes result in diagnostic unclarity. Furthermore, infection is only one of the protean conditions that can lead to SIRS, the rest being sterile inflammation. Currently available standard laboratory signs of sepsis such as leukocytes, lactate, blood glucose and thrombocyte counts are non-specific. In about one-third of sepsis patients, the causative organism fails to be identified, further hampering early commencement of antimicrobial therapy or even worse, the liberal use of board-spectrum antibiotics which would perpetuate resistance to antimicrobial drugs.

Previous research to identify sepsis biomarkers such as cytokines, chemokines, acute phase proteins, soluble receptors and cell surface markers did not reliably differentiate between infectious from non-infectious causes of inflammation. It is a difficult to derive accurate biomarkers for diagnosis of sepsis because a host response of SIRS and to infection is regulated by multiple pathways, complicating efforts to derive accurate biomarkers. Furthermore, the number of useful prognostic biomarkers available is also very low.

Therefore, there is a need for robust, effective biomarkers or a biomarker for diagnosis and/or prognosis of sepsis, and states in the sepsis continuum, that overcome(s), or at least alleviate(s), the above-mentioned problems.

### SUMMARY OF THE INVENTION

The present invention seeks to provide novel methods for detection and/or prognosis of sepsis, and states in the sepsis continuum, in a subject to ameliorate some of the difficulties with, and complement the current methods of detection and/or prediction of sepsis. The present invention further seeks to provide kits for detection and/or prognosis of sepsis, and states in the sepsis continuum, in a subject.

The present invention also seeks to provide novel methods for assessing and/or predicting the severity of sepsis in a subject tested positive for sepsis. Preferably, the methods are for assessing whether a subject has, or is at risk of developing, one of a plurality of conditions selected from infection, mild sepsis and severe sepsis, and/or one of a plurality of conditions selected from the states in the sepsis continuum. The present invention further seeks to provide kits for assessing and/or predicting the severity of sepsis in a subject tested positive for sepsis.

The present invention is based on a multi-gene signature approach as a diagnostic biomarker derived from gene expression profiling in leukocytes isolated from patient blood samples, which provides a diagnostic that is significantly more accurate and proleptic than existent methods. The diagnostic biomarker comprising a set of genes collectively reflect broad-range and convergent effects of inflammatory responses, hormonal signaling, onset of endothelial dysfunction, blood coagulation, organ injury and the like.

The present invention relates to a set of genes which has been derived from a microarray genome wide expression profile, validated by qPCR assay. Surprisingly, hierarchical clustering of the microarray gene expression profiling results demonstrated significant differences in gene expression pattern of leukocytes among the different states in the sepsis continuum, namely, control, infection, non-infected Systemic Inflammatory Response Syndrome (SIRS) or also known as SIRS without infection, sepsis, severe sepsis, cryptic shock and septic shock patients. Differentially expressed genes during sepsis were derived from microarray gene profiling, and a panel of genes were shortlisted from the initial 33,000. Furthermore and surprisingly, analytical validation using qPCR indicates that this panel of genes or biomarkers is progressively dysregulated, such as up- or down-regulation, in subjects across the sepsis continuum, which correlates to microarray results. Gene expression changes in leukocytes can be clearly observed and utilized for diagnosis and/or prognosis of sepsis and states in the sepsis continuum.

In addition to the above, surprisingly, any number of the predetermined panel of genes or biomarkers can be used, and in any combination, for the diagnosis and/or prognosis of sepsis and the states in the sepsis continuum.

In accordance with a first aspect of the invention, there is provided a method of detecting or predicting sepsis in a subject, the method comprising:
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein a difference between the level measured in the first sample and the reference level is indicative of sepsis being present in the first sample.

Preferably, the presence of sepsis is determined by detecting in the subject an increase in the level of the at least one biomarker measured in the first sample, the at least one biomarker selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, as compared to the reference level of the corresponding biomarker.

Preferably, the presence of sepsis is determined by detecting in the subject a decrease in the level of the at least one biomarker measured in the first sample, the at least one biomarker selected from a group consisting of: (a) a polynucleotide comprising a nucleotide sequence set forth in any one of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, as compared to the reference level of the corresponding biomarker.

Preferably, the reference level is the level of the corresponding biomarker in a second sample isolated from at least one subject with no sepsis.

Preferably, the comparing step comprises applying a decision rule to determine or predict the presence or absence of sepsis in the subject.

In accordance with a second aspect of the invention, there is provided a method of detecting or predicting whether a subject has one of a plurality of conditions selected from a group consisting of; control, infection, non-infected systemic inflammatory response syndrome (SIRS), mild sepsis, severe sepsis, septic shock and cryptic shock, the method comprising:
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide Comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein the level measured in the first sample is statistically substantially similar to the reference level is indicative of whether the subject has one of the conditions.

Preferably, the reference level is the level of a corresponding biomarker in a second sample isolated from at least one subject selected from a group consisting of: a control subject, an infection positive subject, a non-infected SIRS positive subject, a mild sepsis positive subject, a severe sepsis positive subject and a cryptic shock positive subject.

Preferably, the comparing step comprises applying a decision rule to determine or predict whether the subject has one of the conditions.

In accordance with a third aspect of the invention, there is provided a kit for performing the method of the first aspect, the kit comprising:
i. at least one reagent capable of specifically binding to the at least one biomarker to quantify the level of the biomarker in the first sample of a subject; and
ii. a reference standard indicating the reference level of the corresponding biomarker.

Preferably, the at least one reagent comprises at least one antibody capable of specifically binding to the at least one biomarker.

Preferably, the kit further comprises at least one additional reagent capable of specifically binding at least one additional biomarker in the first sample, and a reference standard indicating a reference level of a corresponding at least one additional biomarker.

In accordance with a fourth aspect of the invention, there is provided a kit for performing the method of the second aspect, the kit comprising:
i. at least one reagent capable of specifically binding to the at least one biomarker to quantify the level of the biomarker in the first sample of a subject; and
ii. a reference standard indicating the reference level of the corresponding biomarker.

Preferably, the at least one reagent comprises at least one antibody capable of specifically binding to the at least one biomarker.

Preferably, the kit further comprises at least one additional reagent capable of specifically binding at least one additional biomarker in the first sample, and a reference standard indicating a reference level of a corresponding at least one additional biomarker.

In accordance with a fifth aspect of the invention, there is provided a kit for detecting or predicting sepsis in a subject, comprising an antibody capable of binding selectively to at least one biomarker in a first sample isolated from the subject and reagents for detection of a complex formed between the antibody and complement component of the at least one biomarker, wherein the at least one biomarker is selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, and a reference standard indicating a reference level of a corresponding biomarker, wherein a difference between a level of the at least one biomarker measured in the first sample and the reference level is indicative of sepsis being present in the first sample.

Preferably, the reference level is the level of the corresponding biomarker in a second sample isolated from at least one subject with no sepsis.

In accordance with a sixth aspect of the invention, there is provided a kit for detecting or predicting whether a subject has one of a plurality of conditions selected from a group consisting of: control, infection, non-infected systemic inflammatory response syndrome (SIRS), mild sepsis, severe sepsis, septic shock and cryptic shock, comprising an antibody comprising capable of binding selectively to at least one biomarker in a first sample isolated from the subject and reagents for detection of a complex formed between the antibody and complement component of the at least one biomarker, wherein the at least one biomarker is selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, and a reference standard indicating a reference level of a corresponding biomarker, wherein a level of the at least one biomarker measured in the first sample is statistically substantially similar to the reference level is indicative of whether the subject has one of the conditions.

Preferably, the reference level is the level of a corresponding biomarker in a second sample isolated from at least one subject selected from a group consisting of: a control subject, an infection positive subject, a non-infected SIRS positive subject, a mild sepsis positive subject, a severe sepsis positive subject and a cryptic shock positive subject.

In accordance with a seventh aspect of the invention, there is provided a method of detecting or predicting sepsis in a subject, the method comprising:
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide comprising a nucleotide sequence set forth in any one or more, and in any combination, of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO 19, SEQ ID NO: 20, SEQ ID NO 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one or more, and in any combination, of the sequences of (a), that encodes a polypeptide comprising the corresponding amino acid sequence; and (c) a polynucleotide comprising a nucleotide sequence capable of hybridising selectively to any one or more of the sequences of (a), (b), or a complement thereof,
wherein a difference between the level measured in the first sample and the reference level is indicative of sepsis being present in the first sample.

In accordance with an eighth aspect of the invention, there is provided a method of detecting or predicting whether a subject has one of a plurality of conditions selected from a group consisting of: control, infection, non-infected systemic inflammatory response syndrome (SIRS), mild sepsis, severe sepsis, septic shock and cryptic shock, the method comprising:
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide comprising a nucleotide sequence set forth in any one or more, and in any combination, of SEQ. ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one or more, and in any combination, of the sequences of (a), that encodes a polypeptide comprising the corresponding amino acid sequence; and (c) a polynucleotide comprising a nucleotide sequence capable of hybridising selectively to any one or more of the sequences of (a), (b), or a complement thereof,
wherein the level measured in the first sample is statistically substantially similar to the reference level is indicative of whether the subject has one of the conditions.

In accordance with another aspect of the present invention, there is provided at least one gene selected from a predetermined panel of genes for diagnosis of sepsis in a subject.

Another aspect of the present invention provides at least one gene selected from a predetermined panel of genes for prognosis of sepsis in a subject.

Another aspect of the present invention provides a method for detecting, or predicting, sepsis in a subject. The method generally comprises measuring the level of at least one sepsis continuum marker expression product of at least one gene selected from a predetermined panel of genes in a suitable fluid sample obtained from the subject and comparing the level measured to the level of a corresponding sepsis continuum marker expression product in at least one control subject, the control subject being a normal subject, wherein a difference between the level of the at least one sepsis continuum marker expression product and the level of the corresponding sepsis continuum marker expression product is indicative of sepsis being present in the subject.

Another aspect of the present invention provides a method for assessing whether a subject has one of a plurality of conditions selected from infection, mild sepsis and severe sepsis. The method generally comprise the steps of measuring the level of at least one sepsis continuum marker expression product of at least one gene selected from a predetermined panel of genes in a suitable fluid sample obtained from the subject and comparing the level measured to the level of a corresponding sepsis continuum marker expression product in a plurality of control subjects, the control subjects being at least one infection positive subject, at least one mild sepsis positive subject and at least one severe sepsis positive subject, wherein when the level of the at least one expression product is statistically substantially similar to the level of the corresponding sepsis continuum marker expression product of any one of the control subjects, it is indicative of whether the subject has one of the conditions.

Another aspect of the invention provides a kit for detection and/or prognosis of sepsis in a subject, comprising an antibody capable of binding selectively to at least one sepsis continuum marker expression product of at least one gene selected from a predetermined panel of genes in a suitable fluid sample obtained from the subject and reagents for detection of a complex formed between the antibody and a complement component of the at least one expression product.

Another aspect of the invention provides a kit for assessing and/or predicting the severity of sepsis in a subject, comprising an antibody capable of binding selectively to at least one sepsis continuum marker expression product of at least one gene selected from a predetermined panel of genes in a suitable fluid sample obtained from the subject and reagents for detection of a complex formed between the antibody and a complement component of the at least one expression product.

Preferably, the kit is for assessing whether a subject has, or is at risk of developing, one of a plurality of conditions selected from infection, mild sepsis and severe sepsis.

Advantageously, the at least one gene is selected from a predetermined panel of genes comprising of: Homo sapiens acyl-CoA synthetase long-chain family member 1 (ACSL1) gene, Homo sapiens annexin A3 (ANXA3) gene, Homo sapiens cysteine-rich transmembrane module containing 1 (CYSTM1) gene, Homo sapiens chromosome 19 open reading frame 59 (C19orf59) gene, Homo sapiens colony stimulating factor 2 receptor, beta, low-affinity (granulocyte-macrophage) (CSF2RB) gene, Homo sapiens DEAD (Asp-Glu-Ala-Asp) box polypeptide 60-like (DDX60L) gene, Homo sapiens Fc fragment of IgG, high affinity Ib, receptor (CD64) (FCGR1B) gene, Homo sapiens free fatty acid receptor 2 (FFAR2) gene, Homo sapiens formyl peptide receptor 2 (FPR2) gene, Homo sapiens heat shock 70 kDa protein 1B (HSPA1B) gene, Homo sapiens interferon induced transmembrane protein 1 (IFITM1) gene, Homo sapiens interferon induced transmembrane protein 3 (IFITM3) gene, Homo sapiens interleukin 1, beta (IL1B) gene, Homo sapiens interleukin 1 receptor antagonist (IL1 RN) gene, Homo sapiens leukocyte immunoglobulin-like receptor, subfamily A (with TM domain), member 5 (LILRA5) gene, Homo sapiens leucine-rich alpha-2-glycoprotein 1 (LRG1) gene, Homo sapiens myeloid cell leukemia sequence 1 (BCL2-related) (MCL1) gene, Homo sapiens NLR family, apoptosis inhibitory protein (NAIP) gene, Homo sapiens nuclear factor, interleukin 3 regulated (NFIL3) gene, Homo sapiens 5′-nucleotidase, cytosolic Ill (NT5C3) gene, Homo sapiens 6-phosphofructo-2-kinase/fructose-2,6-biphosphatase 3 (PFKFB3) gene, Homo sapiens phospholipid scramblase 1 (PLSCR1) gene, Homo sapiens prokineticin 2 (PROK2) gene, Homo sapiens RAB24, member RAS oncogene family (RAB24) gene, Homo sapiens S100 calcium binding protein Al2 (S100Al2) gene, Homo sapiens selectin L (SELL) gene, Homo sapiens solute carrier family 22 (organic cation/ergothioneine transporter), member 4 (SLC22A4) gene, Homo sapiens superoxide dismutase 2, mitochondrial (SOD2) gene, Homo sapiens SP100 nuclear antigen (SP100) gene, Homo sapiens toll-like receptor 4 (TLR4) gene, Homo sapiens chemokine (C-C motif) ligand 5 (CCL5) gene, Homo sapiens chemokine (C-C motif) receptor 7 (CCR7) gene, Homo sapiens CD3d molecule, delta (CD3-TCR complex) (CD3D) gene, Homo sapiens CD6 molecule (CD6) gene, Homo sapiens Fas apoptotic inhibitory molecule 3 (FAIM3) gene, Homo sapiens Fc fragment of IgE, high affinity I, receptor for; alpha polypeptide (FCERIA) gene, Homo sapiens granzyme K (granzyme 3; tryptase II) (GZMK) gene, Homo sapiens interleukin 7 receptor (IL7R) gene, Homo sapiens killer cell lectin-like receptor subfamily B, member 1 (KLRB1) gene, Homo sapiens mal, T-cell differentiation protein (MAL) gene.

Advantageously, the at least one gene selected from the predetermined panel of genes is either up-regulated or down-regulated in a subject with sepsis.

Advantageously, the at least one gene selected from the predetermined panel of genes is progressively up-regulated or down-regulated from control and SIRS without infection, to infection without SIRS, to mild sepsis to severe sepsis.

Advantageously, any number of the predetermined panel of genes can be selected or used, and in any combination, for the diagnosis and/or prognosis of sepsis.

Advantageously, any number of the predetermined panel of genes can be selected or used, and in any combination, for assessing and/or predicting the severity of sepsis in a subject tested positive for sepsis.

Preferably, the at least one sepsis continuum marker transcript is selected from the group consisting of: (a) a polynucleotide comprising a nucleotide sequence set forth in any one of the sequences listed in List 1; (b) a polynucleotide comprising a nucleotide sequence set forth in any one of the sequences listed in List 1 that encodes a polypeptide comprising its corresponding amino acid sequence.

Advantageously, the present invention can be used to distinguish between patients with no sepsis and patients with sepsis. The present invention can also be used to distinguish patients with sepsis and patients with severe sepsis.

Advantageously, the present invention can be used for the early detection and diagnosis of sepsis, and also the monitoring of patients for an improvement of treatment and outcome for such patients.

Advantageously, the present invention can be used to identify and/or classify a subject or patient as a candidate for sepsis therapy.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention, are as follows.

FIG. 1: Relative average fold change of infection (without SIRS), mild and severe sepsis samples over control by qPCR. (A) 30 up-regulated genes; and (B) 10 down-regulated genes.

FIG. 2: Overlapping genes identified from four different gene classification methods.

FIG. 3: Unsupervised hierarchical clustering heatmap of genes with up- or down-regulated expression level in sepsis continuum.

FIG. 4: Boxplots based on 6 Models (A-F) which allow the stratification of septic/non septic patients. A predetermined cut off between Sepsis/non-sepsis, indicated by the respective horizontal lines, is based on a decision rule for highest total accuracy achievable. For each model a training set based on 100 samples was created (left) and a blinded test of 61 samples was used (right) to validate the models. The Models are:
(A) using 40 genes and HPRT1 as normalization housekeeping gene.
(B) using 8 genes and HPRT1 as normalization housekeeping gene.
(C) using 40 genes and GAPDH as normalization housekeeping gene.
(D) using 8 genes and GAPDH as normalization housekeeping gene.
(E) using 40 genes and both HPRT1 and GAPDH as normalization housekeeping genes.
(F) using 11 genes and both HPRT1 and GAPDH as normalization housekeeping genes.

FIG. 5: Boxplot representing 85 sepsis patients based on either 37 genes (A) or 14 genes (B). Weight scoring system was implemented using 2 models which allow the segregation of severe sepsis from mild sepsis.

FIG. 6: Average plasma protein concentration (S100Al2) in patients selected from the group consisting of control, infection, mild sepsis and severe sepsis/septic shock, indicating a correlation between severity of Sepsis and protein concentration.

### DETAILED DESCRIPTION OF EXEMPLARY EMBODIMENTS

The present invention uses a multi-gene signature approach as a diagnostic biomarker derived from gene expression profiling in leukocytes isolated from blood samples of subjects which provides a diagnostic that is significantly more accurate and faster than existing methods. Advantageously, gene expression profiling overcomes, or at least alleviates, the problem of delayed diagnosis of sepsis as the up- or down-regulation of genes occur before the synthesis of functional gene products such as pro-inflammatory proteins. Advantageously, the present invention can reliably and accurately categorise an individual with sepsis or provide prognostic clues on the progression of the syndrome, thereby allowing for more effective therapeutic intervention.

A cohort study was carried out. The objectives of the cohort study relating to the study of emergency department patients with sepsis include (i) deriving and validating a gene expression panel that are differentially expressed in the leukocytes of patients with and without sepsis to enhance early diagnosis of sepsis; and (ii) investigating the prognostic value of the gene expression panel to guide treatment in sepsis by predicting the severity of sepsis at its onset.

Advantageously, there is provided a method of detecting or predicting sepsis in a subject, the method comprises
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein a difference between the level measured in the first sample and the reference level is indicative of sepsis being present in the first sample.

Advantageously, there is also provided a method of detecting or predicting whether a subject has one of a plurality of conditions selected from a group consisting of: control, infection, non-infected systemic inflammatory response syndrome (SIRS), mild sepsis, severe sepsis, septic shock and cryptic shock, the method comprises
i. measuring the level of at least one biomarker in a first sample isolated from the subject; and
ii. comparing the level measured to a reference level of a corresponding biomarker,
wherein the at least one biomarker is selected from a group consisting of: (a) a polynucleotide comprising a nucleotide sequence set forth in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein the level measured in the first sample is statistically substantially similar to the reference level is indicative of whether the subject has one of the conditions.

As used herein, the singular forms “a”, “an” and “the” include plural referents unless the context clearly dictates otherwise.

The use of “or”, “1” means “and/or” unless stated otherwise. Furthermore, the use of the terms “including” and “having” as well as other forms of those terms, such as “includes”, “included”, “has”, and “have” are not limiting.

“Sample”, “test sample”, “specimen”, “sample used from a subject”, and “patient sample”, including the plural referents, as used herein may be used interchangeably and may be a sample of blood, tissue, urine, serum, plasma, amniotic fluid, cerebrospinal fluid, placental cells or tissue, endothelial cells, leukocytes, or monocytes. The sample can be used directly as obtained from a patient or subject can be pre-treated, such as by filtration, distillation, extraction, concentration, centrifugation, inactivation of interfering components, addition of reagents, and the like, to modify the character of the sample in some manner as discussed herein or otherwise as is known in the art.

Any cell type, tissue, or bodily fluid may be utilised to obtain a sample. Such cell types, tissues, and fluid may include sections of tissues such as biopsy and autopsy samples, frozen sections taken for histological purposes, blood (such as whole blood), plasma, serum, sputum, stool, tears, mucus, saliva, broncholveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynaecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (for example, isolated by another person, at another time, and/or for another purpose). Archival tissues, such as those having treatment or outcome history, may also be used. Protein or nucleotide isolation and/or purification may or may not be necessary.

A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least, about 70%, more usually at least about 80%, preferably at least about 90%, and more preferably at least about 95-98% of the nucleotide bases.

Alternatively, substantial homology or (identity) exists when a nucleic acid or fragment thereof will hybridise to another nucleic acid (or a complementary strand thereof) under selective hybridisation conditions, to a strand, or to its complement. Selectivity of hybridisation exists when hybridisation that is substantially more selective than total lack of specificity occurs. Typically, selective hybridisation will occur when there is at least about 55% identity over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Thus, polynucleotides of the invention preferably have at least 75%, more preferably at least 85%, more preferably at least 90% homology to the sequences shown in List 1 or the sequence listings herein. More preferably there is at least 95%, more preferably at least 98%, homology. Nucleotide homology comparisons may be conducted as described below for polypeptides. A preferred sequence comparison program is the GCG Wisconsin Best fit program described below. The default scoring matrix has a match value of 10 for each identical nucleotide and −9 for each mismatch. The default gap creation penalty is −50 and the default gap extension penalty is −3 for each nucleotide.

In the context of the present invention, a homologue or homologous sequence is taken to include a nucleotide sequence which is at least 60, 70, 80 or 90% identical, preferably at least 95 or 98% identical at the amino acid level over at least 20, 50, 100, 200, 300, 500 or 1000 nucleotides with the nucleotides sequences set out in the sequence listings or in List 1 below. In particular, homology should typically be considered with respect to those regions of the sequence that encode contiguous amino acid sequences known to be essential for the function of the protein rather than non-essential neighbouring sequences. Preferred polypeptides of the invention comprise a contiguous sequence having greater than 50, 60 or 70% homology, more preferably greater than 80, 90, 95 or 97% homology, to one or more of the nucleotides sequences set out in the sequences. Preferred polynucleotides may alternatively or in addition comprise a contiguous sequence having greater than 80, 90, 95 or 97% homology to the sequences set out in the sequence listings or in List 1 below that encode polypeptides comprising the corresponding amino acid sequences.

Other preferred polynucleotides comprise a contiguous sequence having greater than 40, 50, 60, or 70% homology, more preferably greater than 80, 90, 95 or 97% homology to the sequences set out that encode polypeptides comprising the corresponding amino acid sequences.

Nucleotide sequences are preferably at least 15 nucleotides in length, more preferably at least 20, 30, 40, 50, 100 or 200 nucleotides in length.

Generally, the shorter the length of the polynucleotide, the greater the homology required to obtain selective hybridization. Consequently, where a polynucleotide of the invention consists of less than about 30 nucleotides, it is preferred that the % identity is greater than 75%, preferably greater than 90% or 95% compared with the nucleotide sequences set out in the sequence listings herein or in List 1 below. Conversely, where a polynucleotide of the invention consists of, for example, greater than 50 or 100 nucleotides, the % identity compared with the sequences set out in the sequence listings herein or List 1 below may be lower, for example greater than 50%, preferably greater than 60 or 75%.

The “polynucleotide” compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, natural amino acids, etc.), polypeptides with substituted linkages as well as other modifications known in the art, both naturally and non-naturally occurring.

In the context of the present invention, a homologous sequence is taken to include an amino acid sequence which is at least 60, 70, 80 or 90% identical, preferably at least 95 or 98% identical at the amino acid level over at least 20, 50, 100, 200, 300 or 400 amino acids with the sequences set out in the sequence listings or in List 1 below that encode polypeptides comprising the corresponding amino acid sequences. In particular, homology should typically be considered with respect to those regions of the sequence known to be essential for the function of the protein rather than non-essential neighbouring sequences. Preferred polypeptides of the invention comprise a contiguous sequence having greater than 50, 60 or 70% homology, more preferably greater than 80 or 90% homology, to one or more of the corresponding amino acids.

Other preferred polypeptides comprise a contiguous sequence having greater than 40, 50, 60, or 70% homology, of the sequences set out in the sequence listings or in List 1 below that encode polypeptides comprising the corresponding amino acid sequences. Although homology can also be considered in terms of similarity (i.e. amino acid residues having similar chemical properties/functions), in the context of the present invention it is preferred to express homology in terms of sequence identity. The terms “substantial homology” or “substantial identity”, when referring to polypeptides, indicate that the polypeptide or protein in question exhibits at least about 70% identity with an entire naturally-occurring protein or a portion thereof, usually at least about 80% identity, and preferably at least about 90 or 95% identity.

Homology comparisons can be conducted by eye, or more usually, with the aid of readily available sequence comparison programs. These commercially available computer programs can calculate % homology between two or more sequences.

Percentage (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Best fit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Best fit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

Preferred polypeptides of the invention have substantially similar function to the sequences set out in the sequence listings or in List 1 below. Preferred polynucleotides of the invention encode polypeptides having substantially similar function to the sequences set out in the sequence listings or in List 1 below. “Substantially similar function” refers to the function of a nucleic acid or polypeptide homologue, variant, derivative or fragment of the sequences set out in the sequence listings or in List 1 below, with reference to the sequences set out in the sequence listings or in List 1 below or the sequences set out in the sequence listings or in List 1 below that encode polypeptides comprising corresponding amino acid sequences.

Nucleic acid hybridisation will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30 degrees Celsius, typically in excess of 37 degrees Celsius, and preferably in excess of 45 degrees Celsius. Stringent salt conditions will ordinarily be less than 1000 mM, typically less than 500 mM, and preferably less than 200 mM. However, the combination of parameters is much more important than the measure of any single parameter. An example of stringent hybridization conditions is 65° C. and 0.1×SSC (1×SSC=0.15 M NaCl, 0.015 M sodium citrate pH 7.0).

“Subject”, “patient”, and “individual” including the plural referents, as used herein may be used interchangeably and refers to any vertebrate, including but not limited to a mammal. In some embodiments, the subject may be a human or a non-human. The subject or patient may or may not be undergoing other forms of treatment.

“Control” or “controls” as used herein refers to any condition unrelated to any infective cause; no underlying chronic inflammatory condition, autoimmune disease or immunological disorder, for example, asthma, rheumatoid arthritis, inflammatory bowel disease, systemic lupus erythematosus (SLE), type I diabetes mellitus, and the like.

“Systemic Inflammatory Response Syndrome (hereinafter referred to as “SIRS”) without infection” or “non-infected SIRS” as used herein fulfils at least two of the four SIRS criteria (see Table 2 below), and there is no clinical/radiological evidence of infection.

“Infection without SIRS” and “infection” as used herein, may be used interchangeably, does not fulfil at least two of the four SIRS criteria in Table 2 below. There is also clinical/radiological suspicion or confirmation of infection. Patients with such a condition may present symptoms and signs of upper respiratory tract infection/chest infection/pneumonia (including productive cough, runny nose, sore throat, infiltrates on the chest X-ray), urinary tract infection (including cloudy urine, dysuria, positive nitrites in the urinalysis), gastroenteritis (including diarrhoea, vomiting, abdominal cramps), cellulitis/abscess (including redness, swelling, pain, erythema of skin).

“Mild sepsis” as used herein fulfils at least two of the four SIRS criteria in Table 2 below, and there is clinical/radiological suspicion or confirmation of infection. The term also refers to SIRS with infection.

“Severe sepsis” as used herein refers to sepsis with serum lactate >2 mmol/L or evidence of >1 organ dysfunction (see Table 3 below).

“Cryptic shock” as used herein refers to sepsis with serum lactate >4 mmol/L without hypotension.

“Septic shock” as used herein refers to sepsis with hypotension despite 1 litre infusion of intravenous crystalloid.

“States” or “conditions” of the sepsis continuum as used herein refers to control, infection (without SIRS), SIRS without infection, mild sepsis, severe sepsis, cryptic shock and septic shock. “Sepsis” as used herein refers to one or more of the states or conditions comprising mild sepsis, severe sepsis, cryptic shock and septic shock. For example, if a subject is said to have sepsis, or predicted to have sepsis, the subject may be suffering from mild sepsis, or severe sepsis, or cryptic shock or septic shock. “Non-sepsis” or “no sepsis” as used herein refers to one or more of the states or conditions comprising control, infection and SIRS without infection. For example, if a subject is said to have no sepsis, the subject may be a control or has an infection or has SIRS without infection.

“Predetermined cut off” or “cut off” including the plural referents, as used herein refers to an assay cut off value that is used to assess diagnostic, prognostic, or therapeutic efficacy results by comparing the assay results against the predetermined cut off/cut off, where the predetermined cut off/cut off already has been linked or associated with various clinical parameters (for example, presence of disease/condition, stage of disease/condition, severity of disease/condition, progression, non-progression, or improvement of disease/condition, etc.). The disclosure provides exemplary predetermined cut offs/cut offs. However, it would be appreciated that cut off values may vary depending on the nature of the assay (for example, antibodies employed, reaction conditions, sample purity, etc.). Furthermore, it would be appreciated that the disclosure herein may be adapted for other assays, such as immunoassays to obtain immunoassay-specific cut off values for those other assays based on the description provided by this disclosure. Whereas the precise value of the predetermined cut off/cut off may vary between assays, the correlations as described herein should be generally applicable.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, biotechnology, statistics and protein and nucleic acid chemistry and hybridisation described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 1. Materials and Methods

### 1.1. Patient Cohort

A cohort study of patients along with the entire sepsis continuum in the National University Hospital of Singapore (“NUH”), Emergency Department (“ED”) was carried out. Admitted patients were followed-up in the inpatient units. Healthy controls and those with SIRS but without evidence of infection were also recruited to demonstrate differentiation of biomarkers for early diagnosis.

Subjects identified to fulfill the inclusion criteria for recruitment were approached to participate in this study. After informed consent was obtained from subjects, 12 mL of blood was extracted into EDTA tubes and transported on ice to Acumen Research Laboratories (“ARL”). Samples were processed for RNA isolation within 30 minutes after blood collection. Patients who were discharged directly from the ED were tracked for any clinical recurrence of their disease within 30 days to ensure the diagnostic accuracy of the sample of biomarkers that are extracted. All patients that enrolled into the study were followed up after 30 days for final review, to ensure the diagnostic accuracy at recruitment.

Table 1 below shows the inclusion criteria for recruitment of subjects for the cohort study.

TABLE 1
Inclusion criteria (adults 21 years and above) for
patients into categories in sepsis continuum.

| Patient | |
|---|---|
| Category | Criteria |
| Controls | Matched for age and gender |
| | Presents to the ED with condition unrelated to any |
| | infective cause; no underlying chronic inflammatory |
| | condition, autoimmune disease or immunological |
| | disorder (e.g. asthma, rheumatoid arthritis, |
| | inflammatory bowel disease, SLE, type I diabetes |
| | mellitus) |
| SIRS without | Fulfils at least 2 of the 4 SIRS criteria (see Table 2) |
| infection | No clinical/radiological evidence of infection |
| Infection | Does not fulfill at least 2 of the 4 SIRS criteria |
| without SIRS | Clinical/radiological suspicion or confirmation of |
| | infection |
| | Patients may present with symptoms and signs of upper |
| | respiratory tract infection/chest infection/pneumonia |
| | (productive cough, runny nose, sore throat, infiltrates on |
| | the chest X-ray), urinary tract infection (cloudy urine, |
| | dysuria, positive nitrites in the urinalysis), |
| | gastroenteritis (diarrhoea, vomiting, abdominal cramps), |
| | cellulitis/abscess (redness, swelling, pain, erythema of |
| | skin) |
| Mild Sepsis | Fulfill at least 2 of the 4 SIRS criteria |
| | Clinical/radiological suspicion or confirmation of |
| | infection |
| Severe | Sepsis with serum lactate >2 mmol/L OR evidence of >1 |
| sepsis | organ dysfunction (see Table 3) |
| Cryptic | Sepsis with serum lactate >4 mmol/L without |
| shock | hypotension |
| Septic shock | Sepsis with hypotension despite 1 litre infusion of |
| | intravenous crystalloid |

The exclusion criteria for recruitment of subjects for the cohort study includes the following: Age below 21 years, known pregnancy, prisoners, do-not-attempt resuscitation status, requirement for immediate surgery, active chemotherapy, haematological malignancy, treating physician deems aggressive care unsuitable, those unable to give informed consent or unable to comply with study requirements.

The four criteria for SIRS are shown in Table 2 below.

TABLE 2
The four criteria for SIRS
Systemic Inflammatory Response Syndrome (SIRS):

1. A temperature >38° C. or <36° C.
2. Respirations >20 breaths/min or partial pressure of CO2 of <32 mmHg
on the arterial blood gas
3. A pulse rate >90 beats/min
4. A white blood cell count >12,000 cells/mm3 or <4,000 cells/mm3

The indicators of organ dysfunction are shown in Table 3 below.

TABLE 3
Indicators of organ dysfunction
Organ dysfunction:

1. PaO2/FiO2 <300
2. Creatinine >176 μmol/L or increase of more than 44 μmol/L from
baseline
3. Platelet <100 × 109/L
4. INR >1.5
5. PTT >60 seconds
6. Total bilirubin >34 μmol/L

1.2. Collection of Blood Samples from Patients

A total of 12 mL of whole blood was drawn from each patient into four EDTA-coated blood collection tubes. Whole blood was transported on ice and RNA isolation was carried out within 30 minutes of sample collection.

### 1.3. RNA Sample Preparation

1.3.1. RNA Extraction from Leukocytes

Leukocyte RNA purification Kit (Norgen Biotek Corporation) was used according to the manufacturer's instruction for leukocytes RNA extraction.

1.3.2. RNA Quality Control and Storage

RNA concentration and quality were determined using Nanodrop 2000 (Thermo Fisher Scientific). The RNA concentration, 260/280 and 260/230 ratios were recorded. The RNA was then stored in RNase and DNAse free cryotube in liquid nitrogen.

A bioanalyzer (Agilent) was used in addition to Nanodrop to check the RNA quality of samples that was used in microarray studies. The RNA Integrity Number (RIN) of each RNA sample was obtained and images produced by the bioanalyzer after each electrophoretic run was analysed.

### 1.4. Pre-Processing and Analysis of Gene Expression Microarray

Whole-genome gene expression microarray was performed on Illumina® Human HT-12 v4 BeadChip. Each array covers more than 47,000 transcripts and known splice variants across the human transcriptome (NCBI RefSeq Release 38).

In brief, 500 ng of total RNA purified from patient blood samples were amplified and labeled using the Illumina TotalPrep RNA Amplification kit (Ambion) according to the manufacturer's instructions. A total of 750 ng of labelled cRNA was then prepared for hybridization to the Illumina Human HT-12 v4 Expression BeadChip. After hybridization, BeadChips were scanned on a BeadArray Reader using BeadScan software v3.2, and the data was uploaded into GenomeStudio Gene Expression Module software v1.6 for further analysis.

Pre-processing and subsequent bioinformatics analyses were performed using R software and lumi package was to adjust background signals, quantile-normalization, and variance-stabilizing transformation of the raw gene expression data.

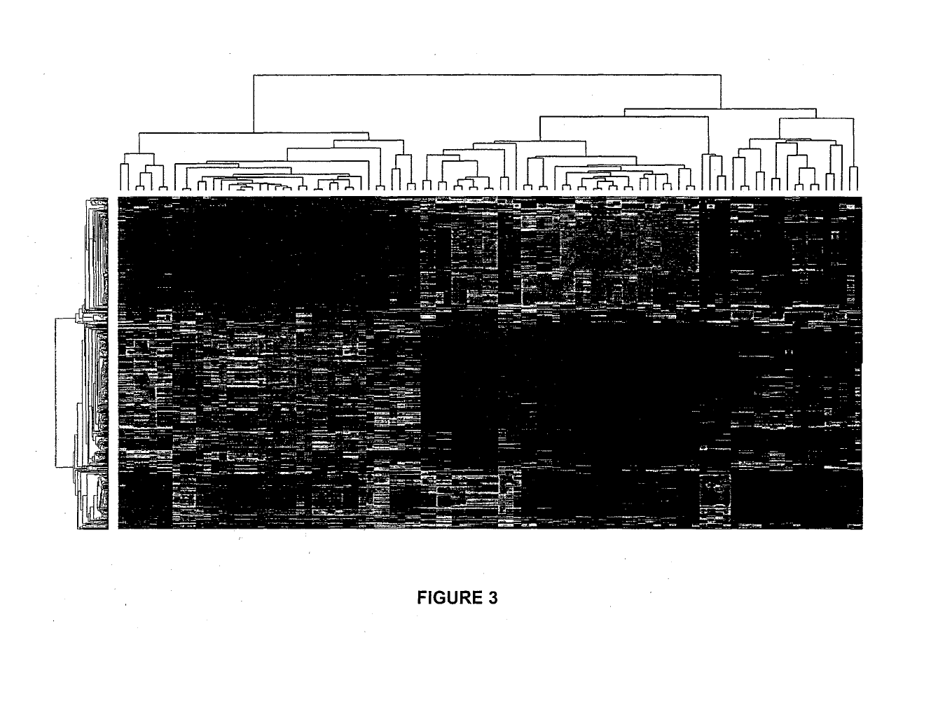

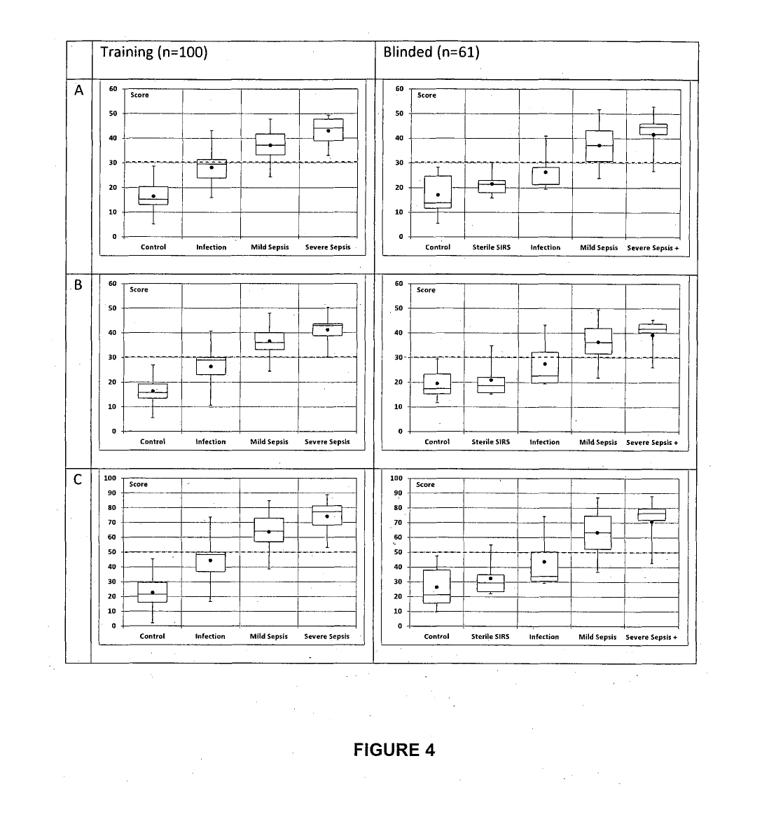

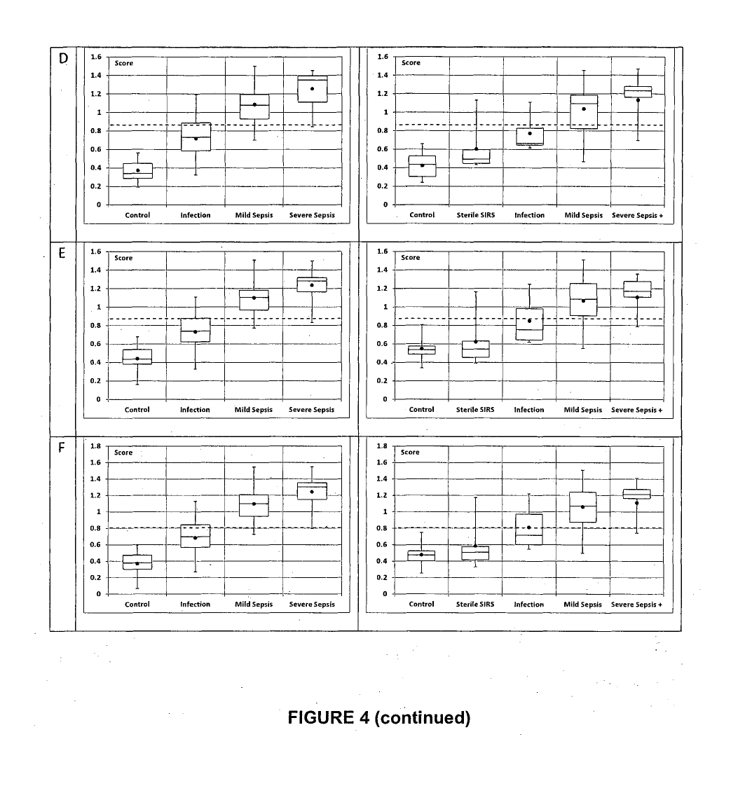

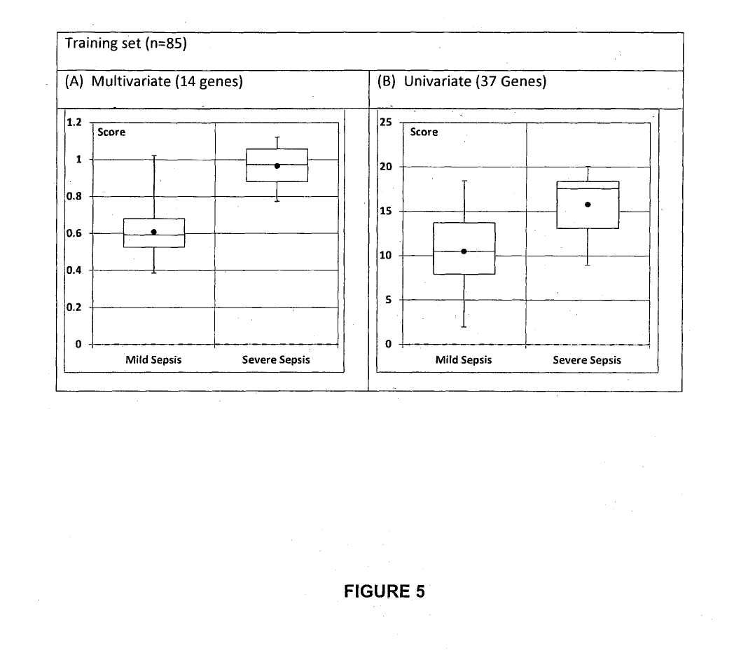

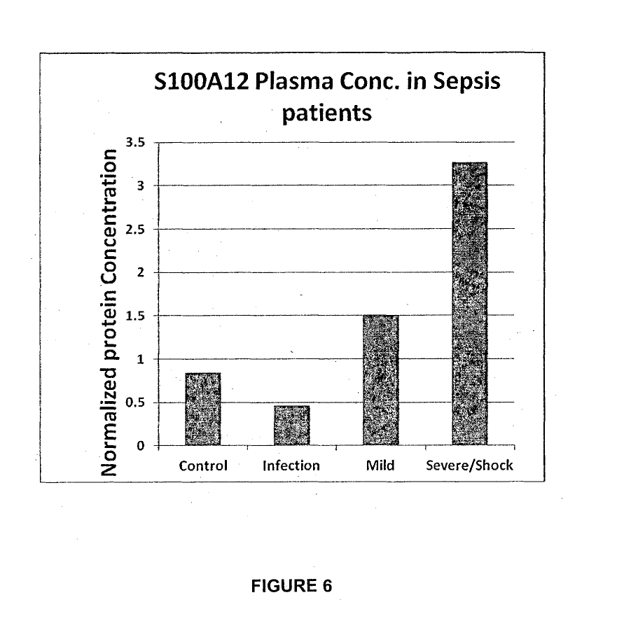

Prior to bioinformatics analyses, quality checks on the microarray were performed. All samples were assessed to possess good RIN quality. Unsupervised hierarchical clustering using Euclidean distance and average linkage revealed highly similar biological replicates (see FIG. 3). After removing potential outliers (n=5) as indicated in FIG. 3, significance analysis of microarray (SAM) was used to select genes that had significantly different expression between sepsis and non-sepsis (fold change >2.0 or <0.5, false discovery rate=0).

A set of significant differentially expressed genes in infection, mild sepsis and severe sepsis were identified through bioinformatics and pathway analyses. Finally, a heat map was generated using Java Treeview to allow visualization of the gene expression profile of each patient group.

1.5. Analytical Validation of Shortlisted Biomarkers by qPCR

1.5.1. cDNA Conversion and Storage

cDNA conversion of RNA samples was performed using iScript™ cDNA Synthesis Kit (Bio-Rad) according to the manufacturer instructions.

1.5.2. Primer Design and Validation

Primers pairs were designed with Primer-BLAST (NCBI, NIH) and Oligo 7. All primer pairs were validated by qPCR for standard curve analysis and in three different RNA samples for melting curve before being shortlisted for additional test in patient samples.

Primer pairs were tested by SYBR Green-based qPCR. Primer pairs that were specific (consistent replicates and single peak in the qPCR melting curve analysis) with strong fold change between infection and mild sepsis subjects (fold change <1.5) were selected. A total of 40 candidate sepsis biomarkers were shortlisted (30 up-regulated genes, 10 down-regulated genes).

Primer pairs were also tested using the standard curve method to determine the efficiencies of qPCR assays (see Table 14). PCR efficiencies were determined using the linear regression slope of template dilution series. Shortlisted biomarkers were required to have efficiency of 80-120% in the linear Ct range (r2>0.99). All 42 primer pairs (40 shortlisted sepsis biomarkers and 2 housekeeping genes) had qPCR efficiency of greater than 80%, which indicate that a standard ddCt method for data analysis is applicable.

1.5.3. Analysis of Shortlisted Biomarkers Expression in Patient Samples by qPCR

Amplification and detection of biomarkers were performed using three systems, LightCycler 1.5 (Roche), LightCycler 480 Instrument I (Roche) and LightCycler 480 Instrument II (Roche). The LightCycler FastStart DNA MasterPlus SYBR Green I Kit (Roche) was used with LightCycler 1.5, while the LightCycler 480 SYBR Green I Master Kit (Roche) was used with LightCycler 480 Instrument I and II (Roche). For both SYBR Green kits, the final reaction volume used was 10 μl with 1 μM working primer concentration and 4.17 μg cDNA template.

All reactions were performed in the following cycling conditions: 95° C. for 10 minutes (initial denaturation); 40-45 cycles of 95° C. for 10 seconds (denaturation), 60° C. for 5 seconds (annealing) and 72° C. for 25 seconds (extension) followed by melting curve analysis and cooling.

Ct values of shortlisted biomarkers were normalized against the housekeeping gene, hypoxanthine phosphoribosyltransferase 1 (HPRT1) and glyceraldehyde-3-phosphate dehydrogenase (GAPDH), to generate ΔCt values for each gene. The relative expression differences between categories in the sepsis continuum (ΔΔCt values) were also calculated. ΔΔCt was then used to calculate the gene expression fold change for each gene. Formulae used are as follows:

ΔCt=Ct biomarker−Ct housekeeping gene

ΔΔCt=Ct sepsis category 1−Ct sepsis category 2

Fold change=2^{-ΔΔCt}

### 1.6. Development and Validation of Predictive Model for Sepsis Diagnosis

A predictive model capable of classifying patients with sepsis from healthy controls that subsequently predict the severity of sepsis was developed. This was performed by training the predictive model using the gene expression (ΔCt values from qPCR) of 46 samples (9 control, 14 SIRS, 14 mild sepsis, and 9 severe sepsis) based on the 40 significant differentially expressed genes. The predictive model was developed with two components, the classification model and regression model, dedicated to the task of diagnosing patients with sepsis, and subsequently predicting sepsis severity respectively.

Ten-fold cross validation was adopted to build and assess five classification models (random forest, decision tree, k-nearest neighbour, support vector machine and logistic regression). The model with highest ten-fold cross validation accuracy is selected (logistic regression) (see Table 4). Similarly, to predict the severity of sepsis, ten-fold cross validation was employed to train and assess different regression models (linear regression, support vector regression, multilayer perceptron, lasso regression, elastic net regression). Likewise, the best-performing regression model in terms of ten-fold cross validation result was selected (support vector regression) (see Table 5).

Table 4 below shows the ten-fold cross validation of five data mining models.

TABLE 4
Ten-fold cross validation of five data mining models

| | | Sensitivity | Specificity | Accuracy |
|---|---|---|---|---|
| Index | Method | (%) | (%) | (%) |
| 1. | RandomForest | 66.7 | 91.9 | 86.96 |
| 2. | J48 (Decision tree) | 55.6 | 89.2 | 82.61 |
| 3. | k-nearest neighbour (k = 2) | 88.9 | 89.2 | 89.13 |
| 4. | Support vector machine | 77.8 | 86.5 | 84.78 |
| | (poly kernel) | | | |
| 5. | Logistic Regression | 77.8 | 91.9 | 89.13 |

Table 5 below shows the ten-fold cross validation of five regression models.

TABLE 5
Ten-fold cross validation of five regression models

| Index | Method | Spearman Rho |
|---|---|---|
| 1. | Linear Regression | 0.8555 |
| 2. | Support Vector Regression | 0.8656 |
| 3. | Multilayer Perceptron | 0.8029 |
| 4. | Lasso Regression | 0.8494 |
| 5. | Elastic Net Regression | 0.8094 |

The predictive model was subjected to a blinded validation process. Twenty four blind samples were used. Prediction of patient sepsis categories was done using the established model. The results were sent to NUH for comparison to clinically assigned categories.

1.7. Development and Validation of a qPCR Multiplex Assay for Detection of Sepsis

1.7.1. Assay Format

Amplification and detection of biomarkers was performed using LightCycler 480 Instrument I (Roche) and LightCycler 480 Instrument II (Roche). Quantifast RT-PCR kit (Qiagen) and LightCycler® 480 Probes Master (Roche) was used. Final reaction volume was 10 μL and 4.17 μg of RNA or cDNA template was used.

For Quantifast RT-PCR kit, reactions were performed with the following cycling conditions: 50° C. for 20 minutes (reverse transcription), 95° C. for 5 minutes (initial denaturation); 40-45 cycles of 95° C. for 15 seconds (denaturation), 60° C. for 30 seconds (annealing and extension), followed by cooling. For LightCycler® 480 Probes Master, reactions were performed with the following cycling conditions: 95° C. for 5 minutes (initial denaturation); 40-45 cycles of 95° C. for 10 seconds (denaturation), 60° C. for 30 seconds (annealing and extension) and 72° C. for 1 second (quantification), followed by cooling.

1.7.2. Taqman Probes Design and Validation

Taqman probes were designed using the Primer3web website (www.primer.wi.mit.edu) and Oligo 7. Autodimer was used to test for dimerization of all primer and probe combinations [1]. All primers-probe were validated in standard curve assay. Primer titration was also performed to determine the lowest primer concentration with consistent Ct value possible.

1.7.3. Validation of Primers-Probe Combinations

Different combinations of primers-probe were tested in multiplex assay using Quantifast RT-PCR+R kit. For 3-plex assay, 0.2 μM primers and 0.2 μM probe for biomarkers were used while 0.4 μM primer and 0.2 μM probe were used for housekeeping gene. A total of 21 3-plex combinations were tested in 8 patient samples. Ct values between 3-plex and monoplex assays were compared. Only the best five 3-plex combinations (average ΔCt difference <1.0 for all component genes and across all sepsis continuum categories) were chosen for further validation.

1.7.4. Nascent 3-Plex Prototype

The best five 3-plex combinations were validated twice in 16 patient samples in Acumen Research Laboratories.

### 2. Results

### 2.1. Patient Cohort

114 subjects were involved in the study: 18 healthy controls, 3 subjects who had SIRS without infection, 30 subjects with infection, 45 subjects with mild sepsis, 15 subjects with severe sepsis and 3 subjects with cryptic shock or septic shock. The demographics and clinical data of subjects are shown in Table 6. The distribution of age, gender, and race were similar across all groups except for SIRS without infection and cryptic/septic shock categories, as both groups had low subject number. There was a male preponderance in the subjects who were recruited

The progression of patients was tracked throughout their hospital stay and for 30 days from initial date of admission to monitor for re-attendance to the ED and re-admission to hospital. There were 6 patients who returned to the ED within 30 days. 2 were for a similar infection as the initial attendance.

Table 6 below shows the subject details grouped accordingly to sepsis continuum.

TABLE 6
Subject details grouped according to sepsis continuum.

| | | | | | | | No. of | |
|---|---|---|---|---|---|---|---|---|
| | | | | | | | patients with | No. of |
| | | | | | | No. of | hospital stay | Patients with |
| | | | | WBC count | Lactate | ICU/HD | between 2-7 | hospital stay |
| Group | Total | Age* | Gender | (×10⁹/L)* | (mmol/L)* | admissions | days | >7 days |
| SIRS without | 3 | 29 (IQR | 33% Male | | | — | — | — |
| infection | | 28-50) | | | | | | |
| Control | 18 | 52.5 (IQR | 61% Male | | | — | — | — |
| | | 48-64) | | | | | | |
| Infection | 30 | 47 (IQR | 63% Male | 7.85 (IQR | 1.2 (IQR | — | 14 | 1 |
| without SIRS | | 38-63) | | 7.05-10.36) | 1-1.7) | | | |
| Mild Sepsis | 45 | 44.5 (IQR | 62% Male | 11.3 (IQR | 1.35 (IQR | 1 | 19 | |
| | | 31-61) | | 8.35-14.89) | 1.05-1.7) | | | |
| Severe sepsis | 15 | 64 (IQR | 73% Male | 11 (IQR | 2.5 (IQR - | — | 10 | 3 |
| | | 54-70) | | 7.44-16.08) | 2.17-2.7) | | | |
| Septic shock | 3 | 65 (IQR | 66% Male | 11.72 (IQR | 5.3 (IQR | 2 | 2 | 1 |
| | | 49-69) | | 11.48-14.35) | 4.1-6.5) | | | |

*Numbers shown indicate the median.
IQR stands for Inter Quartile Range.

### 2.2. Gene Expression Profiling Reveals Potential Markers for Sepsis Diagnosis

In order to identify potential biomarkers that are capable of distinguishing healthy controls and subjects with infection and mild sepsis, whole-genome expression microarray experiments were performed (see Material and Methods above). Significant Analysis of Microarray (SAM) analysis on the gene expression fold change relative to control was conducted to shortlist candidates from the initial ˜33,000 genes on the microarray. Using a stringent thresholds of false discovery rate=0, and fold change >2.0 or <0.5, 444 significantly up-regulated genes and 462 significantly down-regulated genes in sepsis were selected. Many of these identified genes such as ILR1 N, IL1B, TLR1, TNFAIP6 are involved in inflammatory response (p=1.41×10⁻⁵), immune response (p=1.41×10⁻⁵) and wound response (p=1.41×10⁻⁵). This is consistent with the fact that sepsis is a result of an inflammatory response to infection.

### 2.3. Panel of 40 Genes Selected as Sepsis Biomarkers

In order to reduce the list of 906 genes identified through SAM to a clinically feasible number for predictive model development, only the genes with the largest fold change were selected for further testing. In total, eighty five genes were tested, of which eleven were down regulated genes, and 74 were up regulated genes. After qPCR validation, a panel of 40 genes was shortlisted. The panel consists of 30 up-regulated genes and 10 down-regulated genes (see List 1 below).

HRPT1 and GAPDH were selected as the housekeeping genes for their stable expression in leukocytes [2].

List 1 below lists the gene coding sequences for each of the 30 up-regulated genes and 10 down-regulated genes. List 2 below lists the two housekeeping genes.

List 1: Gene coding sequences for each of the 30 up-regulated genes and 10 down-regulated genes

### 30 Up-Regulated Genes

1. ACSL1: Homo sapiens acyl-CoA synthetase long-chain family member 1 (ACSL1), mRNA. NCBI Reference Sequence: NM_001995.2 (SEQ ID NO: 1)
2. ANXA3: Homo sapiens annexin A3 (ANXA3), mRNA. NCBI Reference Sequence: NM_005139.2 (SEQ ID NO: 2)
3. CYSTM1: Homo sapiens cysteine-rich transmembrane module containing 1 (CYSTM1), mRNA. NCBI Reference Sequence: NM_032412.3 (SEQ ID NO: 3)
4. C19orf59: Homo sapiens chromosome 19 open reading frame 59 (C19orf59), mRNA. NCBI Reference Sequence: NM_174918.2 (SEQ ID NO: 4)
5. CSF2RB: Homo sapiens colony stimulating factor 2 receptor, beta, low-affinity (granulocyte-macrophage) (CSF2RB), mRNA. NCBI Reference Sequence: NM_000395.2 (SEQ ID NO: 5)
6. DDX60L: Homo sapiens DEAD (Asp-Glu-Ala-Asp) box polypeptide 60-like (DDX60L), mRNA. NCBI Reference Sequence: NM_001012967.1 (SEQ ID NO: 6)
7. FCGR1B: Homo sapiens Fc fragment a IgG, high affinity Ib, receptor (CD64) (FCGR1B), transcript variant 2, mRNA. NCBI Reference Sequence: NM_001004340.3 (SEQ ID NO: 7)
8. FFAR2: Homo sapiens free fatty acid receptor 2 (FFAR2), mRNA. NCBI Reference Sequence: NM_005306.2 (SEQ ID NO: 8)
9. FPR2: Homo sapiens formyl peptide receptor 2 (FPR2), transcript variant 1, mRNA. NCBI Reference Sequence: NM_001462.3 (SEQ ID NO: 9)
10. HSPA1B: Homo sapiens heat shock 70 kDa protein 1B (HSPA1B), mRNA. NCBI Reference Sequence: NM_005346.4 (SEQ ID NO: 10)
11. IFITM1: Homo sapiens interferon induced transmembrane protein 1 (IFITM1), mRNA. NCBI Reference Sequence: NM_003641.3 (SEQ ID NO: 11)
12. IFITM3: Homo sapiens interferon induced transmembrane protein 3 (IFITM3), transcript variant 1, mRNA. NCBI Reference Sequence: NM_021034.2 (SEQ ID NO: 12)
13. IL1B: Homo sapiens interleukin 1, beta (IL1B), mRNA. NCBI Reference Sequence: NM_000576.2 (SEQ ID NO: 13)
14. IL1RN: Homo sapiens interleukin 1 receptor antagonist (IL1RN), transcript variant 1, mRNA. NCBI Reference Sequence: NM_173842.2 (SEQ ID NO: 14)
15. LILRA5: Homo sapiens leukocyte immunoglobulin-like receptor, subfamily A (with TM domain), member 5 (LILRA5), transcript variant 1, mRNA. NCBI Reference Sequence: NM_021250.2 (SEQ ID NO: 15)
16. LRG1: Homo sapiens leucine-rich alpha-2-glycoprotein 1 (LRG1), mRNA. NCBI Reference Sequence: NM_052972.2 (SEQ ID NO: 16)
17. MCL1: Homo sapiens myeloid cell leukemia sequence 1 (BCL2-related) (MCL1), nuclear gene encoding mitochondrial protein, transcript variant 1, mRNA. NCBI Reference Sequence: NM_021960.4 (SEQ ID NO: 17)
18. NAIP: Homo sapiens NLR family, apoptosis inhibitory protein (NAIP), transcript variant 1, mRNA. NCBI Reference Sequence: NM_004536.2 (SEQ ID NO: 18)
19. NFIL3: Homo sapiens nuclear factor, interleukin 3 regulated (NFIL3), mRNA. NCBI Reference Sequence: NM_005384.2 (SEQ ID NO: 19)
20. NT5C3: Homo sapiens 5′-nucleotidase, cytosolic III (NT5C3), transcript variant 1, mRNA. NCBI Reference Sequence: NM_001002010.2 (SEQ ID NO: 20)
21. PFKFB3: Homo sapiens 6-phosphofructo-2-kinase/fructose-2,6-biphosphatase 3 (PFKFB3), transcript variant 1, mRNA. NCBI Reference Sequence: NM_004566.3 (SEQ ID NO: 21)
22. PLSCR1: Homo sapiens phospholipid scramblase 1 (PLSCR1), mRNA. NCBI Reference Sequence: NM_021105.2 (SEQ ID NO: 22)
23. PROK2: Homo sapiens prokineticin 2 (PROK2), transcript variant 2, mRNA. NCBI Reference Sequence: NM_021935.3 (SEQ ID NO: 23)
24. RAB24: Homo sapiens RAB24, member RAS oncogene family (RAB24), transcript variant 1, mRNA. NCBI Reference Sequence: NM_001031677.2 (SEQ ID NO: 24)
25. S100Al2: Homo sapiens S100 calcium binding protein Al2 (S100Al2), mRNA. NCBI Reference Sequence: NM_005621.1 (SEQ ID NO: 25)
26. SELL: Homo sapiens selectin L (SELL), transcript variant 1, mRNA. NCBI Reference Sequence: NM_000655.4 (SEQ ID NO: 26)
27. SLC22A4: Homo sapiens solute carrier family 22 (organic cation/ergothioneine transporter), member 4 (SLC22A4), mRNA. NCBI Reference Sequence: NM_003059.2 (SEQ ID NO: 27)
28. SOD2: Homo sapiens superoxide dismutase 2, mitochondrial (SOD2), nuclear gene encoding mitochondrial protein, transcript variant 1, mRNA. NCBI Reference Sequence: NM_000636.2 (SEQ ID NO: 28)
29. SP100: Homo sapiens SP100 nuclear antigen (SP100), transcript variant 1, mRNA. NCBI Reference Sequence: NM_001080391.1 (SEQ ID NO: 29)
30. TLR4: Homo sapiens toll-like receptor 4 (TLR4), transcript variant 1, mRNA. NCBI Reference Sequence: NM_138554.4 (SEQ ID NO: 30)

### 10 Down-Regulated Genes

1. CCL5: Homo sapiens chemokine (C-C motif) ligand 5 (CCL5), mRNA. NCBI Reference Sequence: NM_002985.2 (SEQ ID NO: 31)
2. CCR7: Homo sapiens chemokine (C-C motif) receptor 7 (CCR7), mRNA. NCBI Reference Sequence: NM_001838.3 (SEQ ID NO: 32)
3. CD3D: Homo sapiens CD3d molecule, delta (CD3-TCR complex) (CD3D), transcript variant 1, mRNA. NCBI Reference Sequence: NM_000732.4 (SEQ ID NO: 33)
4. CD6: Homo sapiens CD6 molecule (CD6), transcript variant 1, mRNA. NCBI Reference Sequence: NM_006725.4 (SEQ ID NO: 34)
5. FAIM3: Homo sapiens Fas apoptotic inhibitory molecule 3 (FAIM3), transcript variant 1, mRNA. NCBI Reference Sequence: NM_005449.4 (SEQ ID NO: 35)
6. FCER1A: Homo sapiens Fc fragment of IgE, high affinity I, receptor for; alpha polypeptide (FCER1A), mRNA. NCBI Reference Sequence: NM_002001.3 (SEQ ID NO: 36)
7. GZMK: Homo sapiens granzyme K (granzyme tryptase II) (GZMK), mRNA. NCBI Reference Sequence: NM_002104.2 (SEQ ID NO: 37)
8. IL7R: Homo sapiens interleukin 7 receptor (IL7R), mRNA. NCBI Reference Sequence: NM_002185.3 (SEQ ID NO: 38)
9. KLRB1: Homo sapiens killer cell lectin-like receptor subfamily B, member 1 (KLRB1), mRNA. NCBI Reference Sequence: NM_002258.2 (SEQ ID NO: 39)
10. MAL: Homo sapiens mal, T-cell differentiation protein (MAL), transcript variant d, mRNA. NCBI Reference Sequence: NM_022440.2 (SEQ ID NO: 40)
   List 2: Gene coding sequences for each of the two housekeeping genes

### 2 Housekeeping Genes (“HKG”)

1. HPRT1: Homo sapiens hypoxanthine phosphoribosyltransferase 1 (HPRT1), mRNA. NCBI Reference Sequence: NM_000194.2 (SEQ ID NO: 41)
2. GAPDH: Homo sapiens glyceraldehyde-3-phosphate dehydrogenase (GAPDH), mRNA, NCBI Reference Sequence: NM_002046.5 (SEQ ID NO: 42)

### 2.4. Each of the 40 Candidate Sepsis Biomarkers has High Sensitivity and Specificity for Sepsis Diagnosis

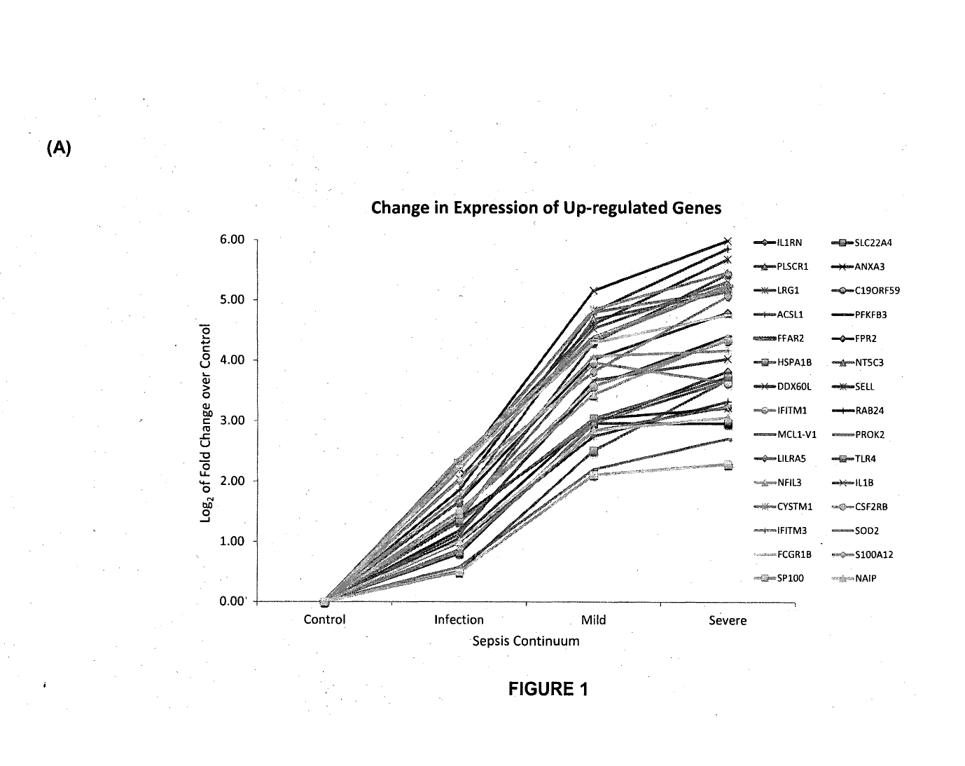

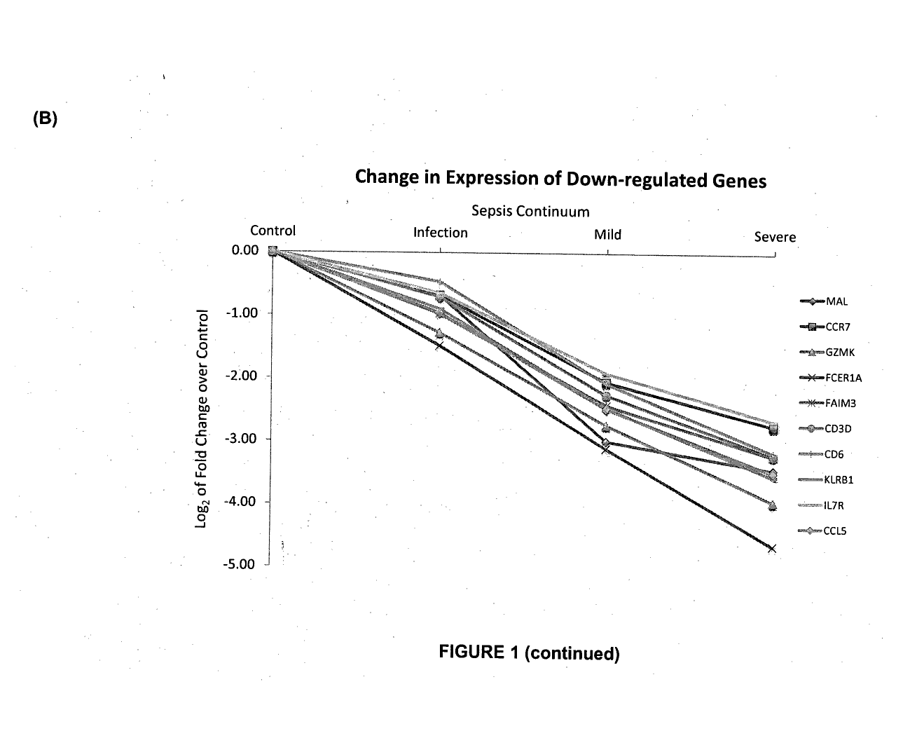

The relative fold change of infection, mild and severe sepsis samples from control samples was compared by qPCR. Progressive up- or down-regulation of gene expression along the sepsis continuum was observed (see FIG. 1). This shows that the selected panel of 40 genes has potential for use in accurately differentiating subject samples along the sepsis continuum.

It is clinically important to distinguish between healthy subjects (controls) from patients with infection (infection, mild sepsis, severe sepsis). The gene panel was tested specifically for the ability to differentiate between controls and infection/mild sepsis/severe sepsis; and between controls/infection from mild sepsis/severe sepsis.

Gene expression fold changes across the sepsis continuum were greater than 1.5, and sufficiently large to be used for differentiation (see Table 15).

The predictive value of each sepsis biomarker was calculated using the Area Under Curve (AUC) of Receiver Operating Characteristic (ROC) curve for differentiation of controls from infection/mild sepsis/severe sepsis and controls/infection from mild sepsis/severe sepsis to ensure that the shortlisted biomarkers have high predictive value for the early differentiation of sepsis (see Table 16). For predictive value when differentiating control from infection/mild/severe, 3 biomarkers had >95%, 18 biomarkers had 90-95% and 16 biomarkers had 85-90%. For predictive value when differentiating control/infection from mild/severe, 10 biomarkers had >95%, 20 biomarkers had 90-95% and 10 biomarkers had 85-90%. p-values are <0.01 for all biomarkers for both differentiation.

### 2.5. Predictive Model Achieved Over 89% Accuracy in Sepsis Diagnosis

A predictive model capable of differentiating between controls and subjects with infection, mild sepsis and severe sepsis was built. The model is an aggregate of two components. The first component (classification model) distinguishes patients with sepsis from controls. If the samples are identified as infection or sepsis, the second component (regression model) will predict the severity of sepsis.

The qPCR gene expression data of the earlier identified 40 differentially expressed genes from 46 samples (9 controls, 14 infection, 14 mild sepsis, and 9 severe sepsis) was used to train the first and second components of the predictive models by using ten-fold cross validation. In each component, different models were tested and the best performing model was selected for that particular component. A logistic regression model was selected as it outperformed the other models tested. It attains a high overall accuracy of 89.13% in classifying sepsis from controls (sensitivity 77.8%, specificity 91.9%) in the ten-fold cross validation assessment.

For the second component, the support vector regression was selected to predict severity of sepsis discovered in the first component. The regression model was capable of accurately predicting the sepsis severity in 87% of the samples.

### 2.6. Predictive Model in Blinded Validation Achieve Accuracy Up to 88% in Sepsis Diagnosis

To further validate the applicability of our model, we performed a blinded assessment using an independent dataset not used in building the predictive models. The 24-sample independent dataset has clinically assessed 3 subjects with SIRS without infection, 4 controls, 2 infection, 12 mild sepsis, 2 severe sepsis and 1 septic shock. For assessment purposes, the subject with septic shock was classified together with severe sepsis.

The predictive model comprises two components with two purposes: diagnosis of sepsis and assessment of sepsis severity. The first component classified sepsis from controls; the selected model has a high overall accuracy of 88%, correctly diagnosing 16 out of 18 subjects with sepsis (sensitivity 94%) and accurately identifying 5 out of 7 controls (specificity 71%). More importantly, the subjects with SIRS without infection were accurately classified as control, showing that the candidate biomarkers were able to differentiate sterile SIRS from sepsis effectively.

The second component is the regression model. Despite the difficulty in predicting severity of sepsis due to the high similarity between infection and mild sepsis, the model was 82% accurate in distinguishing infection from mild sepsis or severe sepsis. This relatively low accuracy indicates the arbitrary threshold for delineation between infection and mild sepsis in the sepsis continuum that is used to guide clinicians to risk stratify patients presenting with illness due to an infective aetiology. Infection, mild sepsis and severe sepsis induce similar inflammatory responses in varying degrees, further increasing the difficulty of making an accurate prediction using the model.

Collectively, these results (see Tables 7 and 8) demonstrate that our approach is not only feasible, but also of good accuracy diagnosing sepsis at an early stage. These results also indicate that refinement of the regression model is needed to better predict the severity of sepsis patients.

Table 7 below shows the performance of biomarker panel for classifying sepsis from control.

TABLE 7
Performance of biomarker panel for classifying sepsis from control

| | Patient | | |
|---|---|---|---|
| | samples | Control | Sepsis |
| Predictions made | 24 | 7 | 17 |
| Control | 6 | 5 | 1 |
| Sepsis | 18 | 2 | 16 |

Table 8 below shows the performance of biomarker panel for staging sepsis severity.

TABLE 8
Performance of biomarker panel for staging sepsis severity

| | Patient | | Mild | Severe |
|---|---|---|---|---|
| | samples | Infection | Sepsis | Sepsis |
| Predictions made | 17 | 2 | 13 | 2 |
| Infection | 5 | 2 | 3 | — |
| Mild | 8 | — | 7 | 1 |
| Severe | 4 | — | 3 | 1 |

2.7. Development and Validation of a qPCR Multiplex Assay for Detection of Sepsis

2.7.1. Development of Multiplex Assay

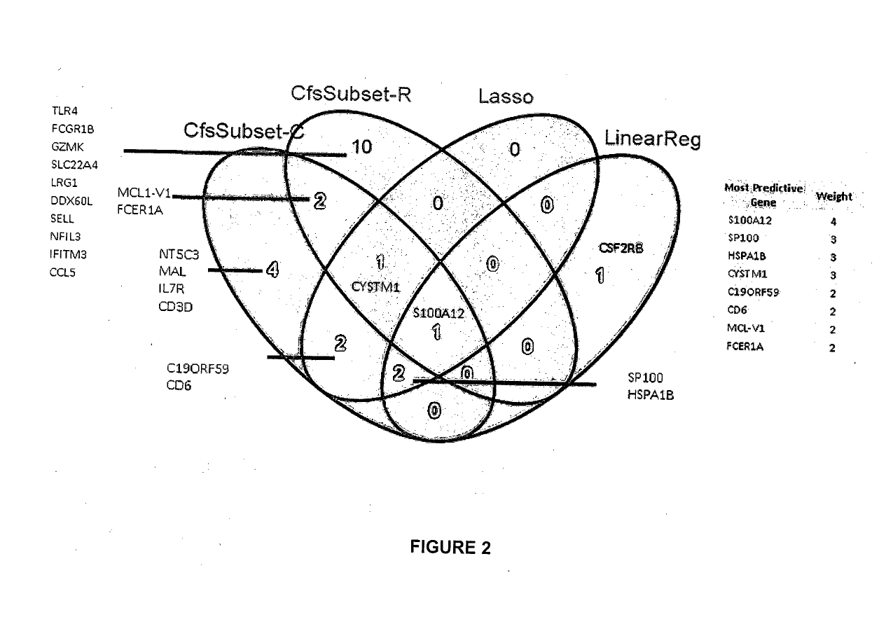

To select the most predictive genes for multiplex development, 10-fold cross validation was performed. From four different 10-fold cross validations of classification methods, 8 recurrent/overlapping genes were identified (see FIG. 2). The overlapping method was chosen because it could reduce bias intrinsic to different classification models which classify data sets according to different assumptions. Concurrently, another 8 genes were selected using predictive value from comparison of control to infection/mild sepsis/severe sepsis using the ROC curve. Selected genes are shown in Table 19 below.

Three-plex combinations were designed from the most predictive genes. A total of 21 combinations of three-plex assays were screened by comparing Ct values in multiplex to monoplex of eight different patient samples (see Table 22). Of the 21 combinations, five three-plex assays had similar Ct values (ΔCt <1.0) and were shortlisted for further validation.

2.7.2. Validation of Multiplex Assay Using Patient Samples

The shortlisted five three-plex assays were tested in additional 8 patient samples. Comparison of Ct value of component genes in multiplex to monoplex assay was made (see Table 23) to determine the validity of the assay. It was observed that only S100Al2/FFAR2/HPRT1 gave consistent result in patient samples from different sepsis categories. MCL1/CYSTM1/HPRT1 was less consistent. In the other three combinations, results were consistent in control samples but not in sepsis samples. The ΔCt of the housekeeping gene, HPRT1, was higher in sepsis samples. This could be due to suppression of HPRT1 amplification by biomarkers that were highly expressed during sepsis.

### 3. Discussion

3.1. Biomarkers from Leukocytes can be Used for Sepsis Diagnosis

Hierarchical clustering of our microarray gene expression profiling results demonstrated significant differences in gene expression pattern of leukocytes between patients with and without infection and sepsis. Differentially expressed genes during sepsis were derived from microarray gene profiling, and a panel genes or biomarkers, in this case 40 genes, were shortlisted from the initial 33,000. The shortlisted panel of genes were validated in qPCR assay. Analytical validation using qPCR have shown that these shortlisted biomarkers were progressively dysregulated in subjects across the sepsis continuum. These results correlated to those obtained from the microarray. Gene expression changes in leukocytes can be clearly observed and potentially utilized for diagnosis and/or prognosis of sepsis and for assessing and/or predicting the severity of sepsis in a subject.

The predictive value of each gene obtained using the AUC of the ROC curve was encouraging, with scores of above 85% for every individual gene. This high predictive value of each gene suggests that the gene panel selected is capable to be utilized as early diagnostic marker. In order to fully leverage on the information from these 40 genes, a predictive model was built using the qPCR ΔΔCT values of all 40 genes. This predictive model was capable of accurately diagnosing 88% of the blind samples. The derived gene expression panel has been shown to be sufficiently distinct across the sepsis continuum to allow immunologic segregation of the subjects along the sepsis continuum that is based on clinical phenotypes.

### 3.2. Exploitation of Biomarkers for Sepsis Diagnosis

Over 33,000 genes were examined through microarray analyses. Using SAM, 906 genes that were differentially expressed across the sepsis continuum were identified and later further reduced to 40 genes. The expression of these 40 genes in all subjects was validated analytically through qPCR where fold change differences were used to build the predictive model.

Predictions made by the model were compared to clinical classifications and a total of 7 mismatched predictions were found. Of the 7 mismatched predictions, 4 of them made no difference to patient management, while 3 could have resulted in adverse outcomes. Despite the small number of SIRS without infection subjects, the model was able to correctly classify both subjects in the blind sample testing. However, further refinement of the model through a subsequent clinical validation phase will have to be carried out to increase its specificity and sensitivity. The panel of genes could potentially be further decreased without sacrificing its accuracy to improve cost efficiency and reproducibility. The use of a larger data set to train the predictive model is paramount to this mission. Other improvements to the system, such as the use of new housekeeping genes to ensure that the baseline used for comparison is stable and able to account for differences in age and gender of the individuals.

### 3.3. Prototyping of Diagnostic Kit

The qualitative gene expression data obtained can be used for multiple applications, including the differentiation of infected and non-infected patients, differentiation of sepsis and non-sepsis patients, and staging severity of sepsis, through the use of different predictive models. Existing data can be merged with new data from future studies for use in new predictive model building. Should it be desirable, new genes can be selected from the microarray data. This could be useful if sufficient information on patient disease progression could be obtained and new genes specifically for use in classifying patient disease prognosis were to be identified. Thus, there is unparalleled flexibility to exploit the data obtained from this study.

Currently, RNA from leukocytes is used as the template for the prototype development. However, starting material for the final prototype may be determined by multiple factors such as processing time and complexity, sensitivity and stability of the assay, equipment available in hospitals, and time taken for sample preparation will have to be considered.

### 3.4. Clinical Utility of Diagnostic Kit

Currently, there is no gold standard for diagnosis of sepsis. Most initial tests rely on positive blood cultures. There are several major drawbacks for relying on blood cultures including the lengthy time required to obtain definitive results (24 to 72 hours), large volume of blood required (usually 20 ml to 40 ml) and false positive rates (0.6% to 10%) [3,4]. Several pathogen-based molecular diagnostic kits have been made commercially available to circumvent this problem, for example, FilmArray® Blood Culture Identification panel (BioFire Diagnostics Inc.). However, this method only identifies the pathogen (and its by-products e.g. endotoxins) that has incited the host inflammatory response and allows targeted anti-microbial therapy to be instituted but does not indicate the collateral damage caused by the over-exuberant host inflammatory response or the severity of sepsis.

The limitation of blood cultures lies also in false negative results which may be caused by low bacterial concentrations in blood, insufficient blood extracted into the culture bottles, presence of fastidious organisms or the use of antibiotics prior to sample collection. Data from NUH ED between 2007 and 2012 showed a true positive blood culture rate of only 21.4% for patients above 65 years old.

The proposed diagnostic kit utilising qPCR assays for the host response in the form of gene expression changes due to infection/sepsis complements the pathogen-based molecular techniques described above. The ability to ascertain a host response for early diagnosis precedes the utilisation of pathogen identification to allow more rapid and accurate management of patients who do not manifest sepsis clinically initially but who may deteriorate later. The pillars of sepsis management including source control, early haemodynamic resuscitation and support, and ventilator support can then be instituted early to improve patient outcomes. The estimated 3 hours required by the gene expression diagnostic kit presents an opportunity for front line doctors such as emergency physicians to make rapid informed decisions for triage and right-siting of care in the hospital.

### 4. Supplementary Methods

### 4.1. Gene Expression Profiling

4.1.1. Quality Control for Comparable Microarray Analysis

Quality control (QC) for microarray hybridization was performed. Control metrics used were hybridization controls for hybridization procedure, low stringency tests for washing temperature, high stringency tests for Cy3 binding, negative controls for non-specific hybridization, gene intensity tests for integrity of samples and amount of hybridization and finally signal distribution analysis to detect outliers.

4.2. Analytical Validation of Shortlisted Biomarkers by qPCR

4.2.1. Primers Design and Validation

The National Centre for Biotechnology Information (NCBI) nucleotide database was used to obtain the coding sequence for each of our selected genes. Primer-BLAST was then run to get 20 different primer pairs for each gene. The parameters used were: 200 bp maximum PCR product size; 20 primer pairs returned; primer melting temperature of minimum 59° C., maximum 61° C. and maximum difference of 2° C. Each pair was then tested for stability and usage in silico using Oligo 7. Top two primer pairs that score more than 700 points were selected for use in qPCR.

Before starting the experiments, each primer pair was tested to check their quality. New primers were tested with three different samples by qPCR. The melting curve was checked to verify that there are no side products or primer dimers. Additionally, standard curve analysis was done to calculate the correlation coefficient (r2) and the efficiency (E) of the primer pairs. The formula used to calculate efficiency is as follows:

E=[−1+10(1/slope)]×100%

The slope was calculated from the standard curve. The validated primer pairs were then used for analytical validation (see Table 9).

Table 9 below shows the list of primers used.

TABLE 9
List of primers used

| Name | Forward primer | Reverse primer |
|---|---|---|
| ACSL1 | GCTCTCGGAAACCAGACCAA | AAGCCCTTCTGGATCAGTGC |
| ANXA3 | GTTGGACACCGAGGAACAGT | CGCTGTGCATTTGACCTCTC |
| C19ORF59 | AACTCCGTACAAGCATGCGA | GGCATTTTCTGCAGCACCTC |
| CSF2RB | CCACGGCCAATACATCGTCT | TTGGTCACGTTGAGGGATGG |
| CYSTM1 | ACCCTACCCACCTCCTCAAG | AGGTGGATGGTCCTAGCTCA |
| DDX60L | CTGAGGACTGCACGTATGCT | TGTAAATCGCACTCGCGGTA |
| FCGR1B | TTGAGGTGTCATGCGTGGAA | TGCCTGAGCAATGGTAGGTG |
| FFAR2 | GGAGTGATTGCAGCTCTGGT | GACCTGCTCAGTCGTGTTCA |
| FPR2 | GGCTACACTGTTCTGCGGAT | CACCCAGATCACAAGCCCAT |
| HSPA1B | CCTGTTTGAGGGCATCGACT | TCGTGAATCTGGGCCTTGTC |
| IFITM1 | CAACATCCACAGCGAGACCT | TCGCCAACCATCTTCCTGTC |
| IFITM3 | CATGTCGTCTGGTCCCTGTT | GTCGCCAACCATCTTCCTGT |
| IL1B | ACCACTACAGCAAGGGCTTC | ATCGTGCACATAAGCCTCGT |
| IL1RN | CCAGCAAGATGCAAGCCTTC | GACTTGACACAGGACAGGCA |
| LILRA5 | GATTCCGGTCTCAGGAGCAG | GAATCCCAAGGACCACCAGG |
| LRG1 | CAGACAGCGACCAAAAAGCC | ATTTCGGCAGGTGGTTGACA |
| MCL1-V1 | AACTGGGGCAGGATTGTGAC | CCCATCCCAGCCTCTTTGTT |
| NAIP | CCTCACGAGACTCCCCATAGA | CGCAAGTCTAGCCTCCTCTT |
| NFIL3 | AGGCCACGCAAAAACTTTCC | TGATGCCAGTGCTCCGATTT |
| NT5C3 | ACAACATAGCATCCCCGTGT | TGAGCACCCCAGTTTCATCA |
| PFKFB3 | AGTGCAGAGGAGATGCCCTA | ATTCCACACGGCAGCCATAA |
| PLSCR1 | CGCCACAGCCTCCATTAAAC | TCCGCTGCAAAGTAAACCCT |
| PROK2 | AGGACTCCCAATGTGGTGGA | TCCCAGTTTGCCCATAGGTG |
| RAB24 | TGCCATCGTCTGCTATGACC | CGCAGTTCCTTCACCCAGAA |
| S100A12 | CGGAAGGGGCATTTTGACAC | TGGTGTTTGCAAGCTCCTTTG |
| SELL | GAACTGGGGAGATGGTGAGC | TAGTTTGTGGCAGGCGTCAT |
| SLC22A4 | GTTCAGCCAGGACGTCTACC | GCACCTTCCAGTTGTCCTCA |
| SOD2 | AAACCTCAGCCCTAACGGTG | GAAACCAAGCCAACCCCAAC |
| SP100 | CTTGCTCACGACCCCAGATT | GGAGCCTTCTCACCATGCTT |
| TLR4 | CATTGGTGTGTCGGTCCTCA | CCAGTCCTCATCCTGGCTTG |
| MAL | CTTGCCCGACTTGCTCTTCA | AGAACACCGCATGGACCAC |
| CCR7 | CTTGTCATCATCCGCACCCT | GAGCTCACAGGTGCTACTGG |
| GZMK | GTTACTACAACGGCGACCCT | AGATTCCAGGCTTTGTGGCA |
| FCER1A | CCAGATGGCGTGTTAGCAGT | TGAAAGGCTGCCATTGTGGA |
| FAIM3 | GAGCCATCATGGGAAGAGCA | GAGTGGTGAACTGGAGGGAC |
| CD3D | GTCTATCAGCCCCTCCGAGA | ACTTGTTCCGAGCCCAGTTT |
| CD6 | ATGAGGAGGTCCAGCAAAGC | AGGTGCTCGACTCACTGTTG |
| KLRB1 | TGAAACTTAGCTGTGCTGGGA | CTCTCGGAGTTGCTGCCAAT |
| IL7R | CCAACCGGCAGCAATGTATG | AGGATCCATCTCCCCTGAGC |
| CCL5 | CAGTCGTCTTTGTCACCCGA | GTTGATGTACTCCCGAACCCA |
| HPRT1 | CCTGGCGTCGTGATTAGTGA | CGAGCAAGACGTTCAGTCCT |
| GAPDH | CCTGGCGTCGTGATTAGTGA | CTCGCTCCTGGAAGATGGTG |

4.3. Development and Validation of a qPCR Multiplex Assay for Detection of Sepsis

4.3.1. Taqman Probes Design and Validation

Taqman probes were designed using the Primer3web website (www.primer.wi.mit.edu) with the following parameters: Probe size was between 18-27 bp; probe melting temperature (Tm) 65-73° C.; GC content 30-80%. Each probe was then tested for stability and usage in silico using Oligo 7. Autodimer was used to test for primer-probe and probe-probe and primer-primer dimerization for all primer and probe combinations [1] (see Table 10).

Table 10 below shows the list of primers-probe combinations.

TABLE 10
List of primers-probe combinations

| Name | Forward primer | Reverse primer | Probe | Fluorophore |
|---|---|---|---|---|
| CYSTM1 | ACCCTACCCACCTCC | AGGTGGATGGTCCTA | TACGGCTGGCAGGGTGGACC | FAM |
| | TCAAG | GCTCA | | |
| IFITM1 | CAACATCCACAGCGA | TCGCCAACCATCTTC | CCGTGCCCGACCATGTCGCT | FAM |
| | GACCT | CTGTC | CTGGTCCC | |
| FFAR2 | GGAGTGATTGCAGCT | GACCTGCTCAGTCGT | TGTCCTTTGGTCACTGCACC | FAM |
| | CTGGT | GTTCA | ATCGTGA | |
| SP100 | CTTGCTCACGACCCC | GGAGCCTTCTCACCA | AGTGAGGAGGAGGCGCCCGC | HEX |
| | AGATT | TGCTT | | |
| IFITM3 | CATGTCGTCTGGTCC | GTCGCCAACCATCTT | ACCCCTGCTGCCTGGGCTTC | HEX |
| | CTGTT | CCTGT | A | |
| SOD2 | AAACCTCAGCCCTAA | GAAACCAAGCCAACC | ACGGCTGCATCTGTTGGTGT | HEX |
| | CGGTG | CCAAC | CCAAGGC | |
| CSF2RB | CCACGGCCAATACAT | TTGGTCACGTTGAGG | GCTCAGTGAACATCCAGATG | Cy5 |
| | CGTCT | GATGG | GCCCC | |
| PROK2 | AGGACTCCCAATGTG | TCCCAGTTTGCCCAT | TGTGCTGTGCTGTCAGTATC | Cy5 |
| | GTGGA | AGGTG | TGGGT | |
| HPRT1 | TCAGGCAGTATAATC | AGTCTGGCTTATATC | CAAGCTTGCTGGTGAAAAGG | Texas Red |
| | CAAAGATGGT | CAACACTTCG | ACCCC | |
| HSPA1B | CCTGTTTGAGGGCAT | TCGTGAATCTGGGCC | AGCACCCTGGAGCCCGTGGA | Cy5 |
| | CGACT | TTGTC | | |
| S100A12 | CGGAAGGGGCATTTT | TGGTGTTTGCAAGCT | AGGGTGAGCTGAAGCAGCTG | LC Cyan 500 |
| | GACAC | CCTTTG | CTTACA | |
| MCL1 | AACTGGGGCAGGATT | CCCATCCCAGCCTCT | TCGTAAGGACAAAACGGGAC | LC Cyan 500 |
| | GTGAC | TTGTT | TGGCT | |

Primer-probe mix was first tested in standard curve assay using serial dilution of template RNA on two different kits: QuantiFast® Multiplex RT-PCR Kit (Qiagen) and LightCycler® 480 Probes Master. (Roche). Sets were validated to ensure that the probe is compatible with primer pairs: the amplification efficiency is within the range of 80-120% and fold change is linear across tested Ct range.

Next, primer titration from 0.4-0.05 μM at 0.05 μM steps was performed to determine the lowest primer concentration possible while maintaining Ct value from the recommended primer concentration of 0.4 μM.

### 5. Supplementary Results

### 5.1. RNA Sample Preparation

5.1.1. RNA Quality and Quantity

The average RNA concentration and ratio for 260/280 and 260/230 acquired for all RNA samples are found. The RNA quality and quantity acquired had concentration >50 ng/uL, 280/260 ratio >2.0, and 260/230 ratio >1.7, showing that good yield was obtained from RNA extraction and RNA samples used were not contaminated with proteins and carbohydrates.

### 5.2. Gene Expression Profiling

5.2.1. RNA Quality and Concentration for Microarray

RNA quality and integrity were tested with Bioanalyzer before being used for microarray experiments. RNA integrity number (RIN) for all samples used in microarray were >7. Electrophoretic runs showed that sharp bands of RNA were present. Results confirmed that RNA samples used in microarray had high integrity and were not degraded.

5.2.2. Quality Control for Microarray Hybridization

Quality control (QC) for microarray hybridization was also performed. Both the pilot (see Table 12) and second microarray (see Table 13) runs passed all quality control tests.

Table 12 below shows the summary of array quality controls for pilot microarrays.

TABLE 12
Summary of array quality controls for the first batch of microarray

| Control Metric | Descriptions | Results |
|---|---|---|
| Hybridization | To QC hybridization | Pass; Signals of hybridization control |
| Controls | procedures | probes met expected values High > |
| | | Medium > Low |
| Low Stringency | To QC hybridization | Pass; Perfect Match probes generated |
| | temperature and high | higher signals than the Mismatch |
| | temperature washing | probes |
| Biotin and | To QC streptavidin-Cy3 | Pass; Biotin-conjugated control probes |
| High Stringency | staining | showed high signals of Cy3 staining |
| Negative Controls | To QC non-specific | Pass; Background signals and noise |
| | hybridization | were at low levels |
| Gene Intensity | To QC integrity of the | Acceptable; Signals of genes were |
| | biological samples and | higher than background and met the |
| | variations in the amount of | expected Housekeeping > All Genes; |
| | samples hybridized | Slight variations in the amount of |
| | | samples hybridized |
| Signal Distribution | Visualization of inter-array | Pass; No outliers identified; Slight |
| (Box Plot) | variations to identify outliers | variations observed as expected |

Table 13 below shows the summary of array quality controls for the second batch of microarray.

TABLE 13
Summary of array quality controls for second microarray

| Control Metric | Descriptions | Results |
|---|---|---|
| Hybridization | To QC hybridization | Pass; Signals of hybridization control |
| Controls | procedures | probes met expected values High > |
| | | Medium > Low |
| Low Stringency | To QC hybridization | Pass; Perfect Match probes generated |
| | temperature and high | higher signals than the Mismatch |
| | temperature washing | probes |
| Biotin and | To QC streptavidin-Cy3 | Pass; Biotin-conjugated control probes |
| High Stringency | staining | showed high signals of Cy3 staining |
| Negative Controls | To QC non-specific | Pass; Background signals and noise |
| | hybridization | were at low levels |
| Gene Intensity | To QC integrity of the | Acceptable; Signals of genes were |
| | biological samples and | higher than background and met the |
| | variations in the amount of | expected Housekeeping > All Genes; |
| | samples hybridized | Slight variations in the amount of |
| | | samples hybridized |
| Signal Distribution | Visualization of inter-array | Pass; No outliers identified; Slight |
| (Box Plot) | variations to identify outliers | variations observed as expected |

5.3. Analytical Validation of Shortlisted Genes by qPCR

5.3.1. Primer Test and Validation

Primer pairs were also tested with the standard curve method to determine the efficiencies of qPCR assays (see Table 14). PCR efficiencies were determined using the linear regression slope of template dilution series. Shortlisted biomarkers were required to have efficiency of 80-120% in the linear Ct range (r² >0.99). Among the 41 primer pairs (40 shortlisted sepsis biomarkers and 1 housekeeping gene), none had qPCR efficiency of <80%. However, 11 primer pairs had efficiency >120%. Despite having >120% efficiency, these primer pairs were still used to study gene expression changes during sepsis since no false products were detected in the melting curve.

Table 14 below shows the efficiency and linear Ct range primer pairs of shortlisted sepsis biomarkers.

TABLE 14
Efficiency and linear Ct range primer
pairs of shortlisted sepsis biomarkers

| No. | Gene name | Efficiency | r² | Linear Ct range | |
|---|---|---|---|---|---|
| 1. | IL1RN | 95% | 0.9974 | 20.57 | 27.49 |
| 2. | SLC22A4 | 109% | — | 30.07 | 33.19 |
| 3. | PLSCR1 | 95% | 0.9997 | 21.63 | 28.53 |
| 4. | ANXA3 | 93% | 0.9987 | 21.41 | 28.40 |
| 5. | LRG1 | 87% | 0.9997 | 27.34 | 34.69 |
| 6. | C19ORF59 | 91% | 0.9860 | 25.71 | 32.84 |
| 7. | ACSL1 | 107% | 0.9969 | 24.18 | 30.52 |
| 8. | PFKFB3 | 96% | 1.0000 | 21.91 | 28.74 |
| 9. | FFAR2 | 124% | 0.9994 | 25.54 | 31.24 |
| 10. | FPR2 | 125% | 0.9990 | 24.6 | 33.13 |
| 11. | HSPA1B | 127% | 0.9983 | 23.12 | 28.73 |
| 12. | NT5C3 | 137% | 0.9944 | 23.70 | 29.03 |
| 13. | DDX60L | 140% | 0.9922 | 23.89 | 29.16 |
| 14. | SELL | 109% | 0.9993 | 22.02 | 31.44 |
| 15. | IFITM1 | 133% | 0.9945 | 20.21 | 28.56 |
| 16. | RAB24 | 134% | 0.9989 | 25.73 | 33.93 |
| 17. | MCL1-V1 | 141% | 0.9984 | 20.48 | 25.72 |
| 18. | PROK2 | 117% | 0.9995 | 21.89 | 27.84 |
| 19. | LILRA5 | 98% | 1.0000 | 22.68 | 29.42 |
| 20. | TLR4 | 122% | 0.9990 | 22.73 | 28.50 |
| 21. | NFIL3 | 123% | 0.9979 | 22.53 | 28.27 |
| 22. | IL1B | 105% | 0.9976 | 23.29 | 29.70 |
| 23. | CYSTM1 | 110% | 0.9991 | 21.47 | 27.69 |
| 24. | CSF2RB | 122% | 0.9998 | 21.83 | 27.95 |
| 25. | IFITM3 | 117% | 0.9990 | 16.11 | 22.07 |
| 26. | SOD2 | 112% | 0.9981 | 19.43 | 25.54 |
| 27. | FCGR1B | 115% | 0.9994 | 21.08 | 27.09 |
| 28. | S100A12 | 96% | 0.9997 | 18.23 | 25.05 |
| 29. | SP100 | 100% | 0.9983 | 21.76 | 28.42 |
| 30. | NAIP | 86% | 0.9979 | 21.17 | 28.61 |
| 31. | MAL | 111% | — | 31.68 | 34.76 |
| 32. | CCR7 | 99% | 0.9993 | 26.99 | 33.66 |
| 33. | GZMK | 85% | 0.9918 | 27.815 | 35.32 |
| 34. | FCER1A | 97% | 0.9990 | 29.205 | 36.00 |
| 35. | FAIM3 | 100% | 0.9997 | 26.925 | 33.55 |
| 36. | CD3D | 91% | 0.9992 | 26.935 | 34.08 |
| 37. | CD6 | 82% | 0.9946 | 28.325 | 36.03 |
| 38. | KLRB1 | 99% | 0.9938 | 27.865 | 34.55 |
| 39. | IL7R | 84% | 0.9802 | 27.14 | 34.70 |
| 40. | CCL5 | 104% | 0.9999 | 25.02 | 31.47 |
| 41. | HRPT1 | 106% | 0.9974 | 26.26 | 32.62 |

5.3.2. Diagnostic Performance of Shortlisted Genes

FIG. 1 shows the relative fold change of infection, mild and severe sepsis samples over control by qPCR. (A) 30 up-regulated genes; and (B) 10 down-regulated genes.

Table 15 below shows the fold change between control versus infection and infection versus mild sepsis. C—control, I—infection, M—mild.

TABLE 15
Fold change between control versus infection and infection
versus mild sepsis C—control, I—infection, M—mild.

| | | Fold change | Fold change |
|---|---|---|---|
| | | Control versus | Infection versus |
| No. | Gene name | Infection | Mild Sepsis |
| 1. | IL1RN | 3.18 | 5.09 |
| 2. | SLC22A4 | 1.14 | 5.47 |
| 3. | PLSCR1 | 3.16 | 8.09 |
| 4. | ANXA3 | 4.57 | 7.77 |
| 5. | LRG1 | 4.64 | 5.21 |
| 6. | C19ORF59 | 2.58 | 7.60 |
| 7. | ACSL1 | 3.62 | 7.69 |
| 8. | PFKFB3 | 2.27 | 5.21 |
| 9. | FFAR2 | 5.10 | 3.98 |
| 10. | FPR2 | 2.62 | 2.97 |
| 11. | HSPA1B | 1.42 | 3.99 |
| 12. | NT5C3 | 1.78 | 4.39 |
| 13. | DDX60L | 2.17 | 5.84 |
| 14. | SELL | 2.07 | 3.95 |
| 15. | IFITM1 | 2.69 | 5.79 |
| 16. | RAB24 | 1.98 | 3.38 |
| 17. | MCL1-V1 | 1.50 | 3.06 |
| 18. | PROK2 | 4.79 | 5.80 |
| 19. | LILRA5 | 1.83 | 3.92 |
| 20. | TLR4 | 2.51 | 3.28 |
| 21. | NFIL3 | 2.83 | 3.81 |
| 22. | IL1B | 4.11 | 5.59 |
| 23. | CYSTM1 | 4.54 | 6.31 |
| 24. | CSF2RB | 2.84 | 4.19 |
| 25. | IFITM3 | 3.39 | 4.94 |
| 26. | SOD2 | 5.21 | 4.02 |
| 27. | FCGR1B | 3.76 | 6.07 |
| 28. | S100A12 | 4.05 | 3.47 |
| 29. | SP100 | 1.41 | 3.06 |
| 30. | NAIP | 2.01 | 3.58 |
| 31. | MAL | 1.61 | 4.92 |
| 32. | CCR7 | 1.60 | 2.59 |
| 33. | GZMK | 2.42 | 2.74 |
| 34. | FCER1A | 2.80 | 3.07 |
| 35. | FAIM3 | 1.97 | 2.72 |
| 36. | CD3D | 1.63 | 2.92 |
| 37. | CD6 | 1.38 | 3.04 |
| 38. | KLRB1 | 1.86 | 2.95 |
| 39. | IL7R | 1.57 | 2.39 |
| 40. | CCL5 | 1.94 | 2.85 |

Table 16 below shows the predictive value (Area Under Curve; AUC), standard deviation and p-value of biomarker panel for control versus infection/mild sepsis/severe sepsis and control/infection versus mild sepsis/severe sepsis.

TABLE 16
Predictive value (Area Under Curve; AUC), standard deviation
and p-value of biomarker panel for control versus infection/mild sepsis/
severe sepsis and control/infection versus mild sepsis/severe sepsis.

| | | Control vs Infection/Mild/Severe | | | Control/Infection vs Mild/Severe | | |
|---|---|---|---|---|---|---|---|
| No. | Gene name | AUC | SD | p-value | AUC | SD | p-value |
| 1. | IL1RN | 90.1% | 4.5% | 0.0002 | 90.2% | 5.2% | <0.0001 |
| 2. | SLC22A4 | 85.6% | 5.5% | 0.0010 | 90.6% | 4.7% | <0.0001 |
| 3. | PLSCR1 | 90.4% | 4.4% | 0.0002 | 95.7% | 3.1% | <0.0001 |
| 4. | ANXA3 | 92.8% | 3.8% | <0.0001 | 95.1% | 2.9% | <0.0001 |
| 5. | LRG1 | 93.1% | 3.8% | <0.0001 | 93.3% | 3.4% | <0.0001 |
| 6. | C19ORF59 | 91.6% | 4.7% | 0.0001 | 96.4% | 2.3% | <0.0001 |
| 7. | ACSL1 | 91.7% | 4.1% | 0.0001 | 94.7% | 3.1% | <0.0001 |
| 8. | PFKFB3 | 88.3% | 4.9% | 0.0004 | 94.7% | 2.9% | <0.0001 |
| 9. | FFAR2 | 94.6% | 3.3% | <0.0001 | 89.1% | 5.0% | <0.0001 |
| 10. | FPR2 | 90.1% | 4.6% | 0.0002 | 89.3% | 4.8% | <0.0001 |
| 11. | HSPA1B | 82.0% | 7.0% | 0.0032 | 88.1% | 5.5% | <0.0001 |
| 12. | NT5C3 | 87.1% | 5.2% | 0.0006 | 91.6% | 4.1% | <0.0001 |
| 13. | DDX60L | 88.0% | 5.2% | 0.0005 | 95.8% | 2.9% | <0.0001 |
| 14. | SELL | 88.9% | 4.9% | 0.0003 | 91.5% | 4.7% | <0.0001 |
| 15. | IFITM1 | 88.6% | 4.8% | 0.0004 | 92.4% | 4.6% | <0.0001 |
| 16. | RAB24 | 89.8% | 4.7% | 0.0002 | 93.6% | 3.5% | <0.0001 |
| 17. | MCL1-V1 | 88.1% | 5.2% | 0.0004 | 95.0% | 3.0% | <0.0001 |
| 18. | PROK2 | 94.0% | 3.5% | <0.0001 | 95.7% | 2.6% | <0.0001 |
| 19. | LILRA5 | 87.7% | 5.1% | 0.0005 | 95.8% | 2.6% | <0.0001 |
| 20. | TLR4 | 92.2% | 4.1% | 0.0001 | 92.6% | 3.6% | <0.0001 |
| 21. | NFIL3 | 92.2% | 4.1% | 0.0001 | 95.1% | 2.8% | <0.0001 |
| 22. | IL1B | 92.5% | 4.0% | <0.0001 | 93.3% | 3.5% | <0.0001 |
| 23. | CYSTM1 | 96.9% | 2.3% | <0.0001 | 97.9% | 1.6% | <0.0001 |
| 24. | CSF2RB | 94.0% | 3.4% | <0.0001 | 93.8% | 3.4% | <0.0001 |
| 25. | IFITM3 | 95.5% | 3.0% | <0.0001 | 96.0% | 2.4% | <0.0001 |
| 26. | SOD2 | 94.9% | 3.1% | <0.0001 | 91.1% | 4.1% | <0.0001 |
| 27. | FCGR1B | 96.3% | 2.6% | <0.0001 | 90.0% | 4.4% | <0.0001 |
| 28. | S100A12 | 94.7% | 3.7% | <0.0001 | 90.2% | 4.4% | <0.0001 |
| 29. | SP100 | 90.4% | 4.4% | 0.0002 | 97.7% | 1.7% | <0.0001 |
| 30. | NAIP | 89.3% | 4.7% | 0.0003 | 91.1% | 4.2% | <0.0001 |
| 31. | MAL | 86.6% | 5.6% | 0.0007 | 94.0% | 3.3% | <0.0001 |
| 32. | CCR7 | 86.2% | 6.1% | 0.0009 | 88.7% | 4.9% | <0.0001 |
| 33. | GZMK | 93.4% | 4.1% | <0.0001 | 88.7% | 5.0% | <0.0001 |
| 34. | FCER1A | 89.8% | 4.9% | 0.0002 | 85.8% | 5.5% | <0.0001 |
| 35. | FAIM3 | 91.9% | 4.2% | 0.0001 | 92:5% | 3.7% | <0.0001 |
| 36. | CD3D | 89.8% | 4.9% | 0.0002 | 92.1% | 4.2% | <0.0001 |
| 37. | CD6 | 84.5% | 5.9% | 0.0015 | 92.6% | 4.0% | <0.0001 |
| 38. | KLRB1 | 88.6% | 5.6% | 0.0004 | 89.4% | 4.8% | <0.0001 |
| 39. | IL7R | 81.7% | 6.9% | 0.0035 | 89.5% | 4.5% | <0.0001 |
| 40. | CCL5 | 89.6% | 5.3% | 0.0003 | 88.2% | 5.3% | <0.0001 |

5.3.3. Derivation of Predictive Model for Differentiation of Sepsis Categories

Weights were given to each gene to generate the logistic regression index were shown (see Table 17). The algorithm used for classifying blind patient sample during clinical validation will be:

Logistic regression index=(dCₜ·w)+I

dCₜ—gene cycle threshold normalized to housekeeping gene
w—weight
I—intercept
For healthy control samples, logistic regression index ≧0
For infected/sepsis samples, logistic regression index <0

Table 17 below shows the weights for each gene and intercept from logistic regression model.

TABLE 17
Weights for each gene and intercept
from logistic regression model.

| No. | Gene name | Weight |
|---|---|---|
| 1. | IL1RN | 2.9035 |
| 2. | SLC22A4 | −1.9025 |
| 3. | PLSCR1 | 6.3155 |
| 4. | ANXA3 | −2.1455 |
| 5. | LRG1 | −0.4864 |
| 6. | C19ORF59 | 0.5169 |
| 7. | ACSL1 | −2.2421 |
| 8. | PFKFB3 | −4.0446 |
| 9. | FFAR2 | −1.5183 |
| 10. | FPR2 | −7.6375 |
| 11. | HSPA1B | −1.4681 |
| 12. | NT5C3 | −2.9469 |
| 13. | DDX60L | −5.1756 |
| 14. | SELL | −3.2046 |
| 15. | IFITM1 | 6.8869 |
| 16. | RAB24 | −1.6036 |
| 17. | MCL1-V1 | −16.5876 |
| 18. | PROK2 | 3.3069 |
| 19. | LILRA5 | −9.2405 |
| 20. | TLR4 | −1.2054 |
| | Intercept | 109.3536 |
| 21. | NFIL3 | −5.9539 |
| 22. | IL1B | −0.9397 |
| 23. | CYSTM1 | 8.7944 |
| 24. | CSF2RB | −0.6782 |
| 25. | IFITM3 | 12.506 |
| 26. | SOD2 | 11.0719 |
| 27. | FCGR1B | 9.6114 |
| 28. | S100A12 | 9.3856 |
| 29. | SP100 | 7.6691 |
| 30. | NAIP | −0.0011 |
| 31. | MAL | 1.7855 |
| 32. | CCR7 | −6.1928 |
| 33. | GZMK | −1.4079 |
| 34. | FCER1A | −7.0497 |
| 35. | FAIM3 | −11.3155 |
| 36. | CD3D | 8.0665 |
| 37. | CD6 | 15.9739 |
| 38. | KLRB1 | −1.2603 |
| 39. | IL7R | 0.8408 |
| 40. | CCL5 | 3.4355 |

Weights were given to each gene to generate the support vector regression index were shown (see Table 18). The algorithm used for classifying blind patient sample during clinical validation will be:

Support vector regression index=(dCₜ·w)+I

dCₜ—gene cycle threshold normalized to housekeeping gene
w—weight
I—intercept
For infection samples, support vector regression index ≧1.41
For mild sepsis samples, support vector regression index 1.41≧x<3.52
For severe sepsis samples, support vector regression index <3.52

Table 18 below shows the weights for each gene and intercept from support vector regression model.

TABLE 18
Weights for each gene and intercept
from support vector regression model.

| No. | Gene name | Weight |
|---|---|---|
| 1. | IL1RN | 0.227 |
| 2. | SLC22A4 | 0.2338 |
| 3. | PLSCR1 | 0.1354 |
| 4. | ANXA3 | 0.0052 |
| 5. | LRG1 | 0.0987 |
| 6. | C19ORF59 | −0.2757 |
| 7. | ACSL1 | −0.145 |
| 8. | PFKFB3 | 0.0545 |
| 9. | FFAR2 | −0.0471 |
| 10. | FPR2 | −0.0067 |
| 11. | HSPA1B | −0.4868 |
| 12. | NT5C3 | −0.3787 |
| 13. | DDX60L | −0.0569 |
| 14. | SELL | 0.1356 |
| 15. | IFITM1 | 0.4329 |
| 16. | RAB24 | −0.1011 |
| 17. | MCL1-V1 | −0.2838 |
| 18. | PROK2 | 0.2847 |
| 19. | LILRA5 | −0.0464 |
| 20. | TLR4 | −0.1839 |
| | Intercept | 0.635 |
| 21. | NFIL3 | 0.1661 |
| 22. | IL1B | 0.0219 |
| 23. | CYSTM1 | −0.0325 |
| 24. | CSF2RB | 0.2387 |
| 25. | IFITM3 | 0.1498 |
| 26. | SOD2 | 0.1162 |
| 27. | FCGR1B | 0.1017 |
| 28. | S100A12 | −0.28 |
| 29. | SP100 | −0.7538 |
| 30. | NAIP | −0.1359 |
| 31. | MAL | 0.0864 |
| 32. | CCR7 | 0.0372 |
| 33. | GZMK | −0.0396 |
| 34. | FCER1A | 0.0254 |
| 35. | FAIM3 | 0.0914 |
| 36. | CD3D | 0.2472 |
| 37. | CD6 | 0.4069 |
| 38. | KLRB1 | −0.0664 |
| 39. | IL7R | 0.1173 |
| 40. | CCL5 | −0.0715 |

5.4. Development and Validation of a qPCR Multiplex Assay for Detection of Sepsis

FIG. 2 shows the most predictive genes identified from overlap of four different classification methods.

Table 19 below shows the list of top eight predictive genes from two different selection methods.

TABLE 19
List of top eight predictive genes from
two different selection methods

| | | | Overlap of |
|---|---|---|---|
| | ROC predictive | | classification |
| No. | value | No. | models |
| 1. | CYSTM1 | 1. | S100A12 |
| 2. | FCGR1B | 2. | SP100 |
| 3. | IFITM3 | 3. | HSPA1B |
| 4. | SOD2 | 4. | CYSTM1 |
| 5. | S100A12 | 5. | C19ORF59 |
| 6. | FFAR2 | 6. | CD6 |
| 7. | PROK2 | 7. | MCL-V1 |
| 8. | CSF2RB | 8. | FCER1A |

Primers-probe was tested with the standard curve method to confirm that primers-probe can produce amplification curves and to determine the efficiencies of qPCR assays. PCR efficiencies were determined using the linear regression slope of template dilution series. Similar to qPCR using SYBR Green format, primers-probe need to have efficiency of 80-120% in the linear Ct range (r² >0.99).

Primers-probe for 12 biomarkers and one housekeeping were designed. Primers-probe of two genes failed to produce amplification curves. Of the 4 housekeeping primer probes, one was chosen for most consistent result. All probes which worked have acceptable efficiency (80-120%) and linear in tested Ct range (see Table 20).

Table 20 below shows the efficiency and linear Ct range primers-probe of tested sepsis biomarkers.

TABLE 20
Efficiency and linear Ct range primers-
probe of tested sepsis biomarkers

| | | | | |
|---|---|---|---|---|
| No. | Gene name | Efficiency | r² | Ct range |

| | | | | | |
|---|---|---|---|---|---|
| 1. | CYSTM1 | 96% | 0.9685 | 26.65 | 37.32 |
| 2. | FFAR2 | 116% | 0.9991 | 24.61 | 30.61 |
| 3. | IFITM1 | 121% | 0.9800 | 20.97 | 29.49 |
| 4. | HPRT1 | 85% | 0.9980 | 28.98 | 36.48 |
| 5. | CSF2RB | 113% | 0.9960 | 23.48 | 32.44 |
| 6. | PROK2 | 117% | 0.9990 | 23.85 | 29.80 |
| 7. | SP100 | 105% | 0.9980 | 25.53 | 35.06 |
| 8. | SOD2 | 121% | 0.9892 | 23.57 | 29.37 |
| 9. | IFITM3 | 108% | 0.9993 | 20.69 | 26.96 |
| 10. | S100A12 | 75% | 0.9984 | 21.81 | 34.25 |
| 11. | MCL1 | 82% | 0.9962 | 19.80 | 31.26 |
| 12. | HSPA1B | 82% | 0.9964 | 23.73 | 35.46 |

Primer titration was performed to reduce the primer concentration used for highly abundant genes (see Table 21). Reduced primer concentration should not be affecting Ct value compared to the recommended starting working concentration of 0.4 uM. Reducing primer concentration will limit the effect of amplification suppression of highly abundant genes on low abundant genes through qPCR reactant competition and depletion. Since, possible minimum final primer concentration ranged from 0.20 to 0.05 μM, 0.2 μM was selected as the final primer concentration for all biomarkers. Final primer concentration for low abundance housekeeping gene was maintained at 0.4 μM.

Table 21 below shows the efficiency and linear Ct range primers-probe of tested sepsis biomarkers.

TABLE 21
Efficiency and linear Ct range primers-
probe of tested sepsis biomarkers.

| | Slope | Titration | Minimum |
|---|---|---|---|
| HPRT1 | 2.01 | Ct up | — |
| CYSTM1 | 0.61 | Stable | 0.10 |
| FFAR2 | 0.24 | Stable | 0.05 |
| SP100 | −0.29 | Stable | 0.05 |
| SOD2 | −1.66 | Ct down | 0.15 |
| IFITM3 | −0.08 | Stable | 0.10 |
| IFITM1 | 1.67 | Ct up | 0.10 |
| CSF2RB | 4.18 | Ct up | 0.10 |
| PROK2 | −3.19 | Ct down | 0.20 |

Table 22 below shows the tested 3-plex combinations.

TABLE 22
Tested 3-plex combinations

| No. | Combinations |
|---|---|
| 1. | CYSTM1/SP100/HPRT1 |
| 2. | CYSTM1/SOD2/HPRT1 |
| 3. | CYSTM1/IFITM3/HPRT1 |
| 4. | FFAR2/SP100/HPRT1 |
| 5. | FFAR2/SOD2/HPRT1 |
| 6. | FFAR2/IFITM3/HPRT1 |
| 7. | IFITM1/SP100/HPRT1 |
| 8. | IFITM1/SOD2/HPRT1 |
| 9. | IFITM1/IFITM3/HPRT1 |
| 10. | MCL1/CYSTM1/HPRT1 |
| 11. | MCL1/FFAR2/HPRT1 |
| 12. | MCL1/IFITM1/HPRT1 |
| 13. | MCL1/SP100/HPRT1 |
| 14. | MCL1/SOD2/HPRT1 |
| 15. | MCL1/IFITM3/HPRT1 |
| 16. | S100A12/CYSTM1/HPRT1 |
| 17. | S100A12/FFAR2/HPRT1 |
| 18. | S100A12/IFITM1/HPRT1 |
| 19. | S100A12/SP100/HPRT1 |
| 20. | S100A12/SOD2/HPRT1 |
| 21. | S100A12/IFITM3/HPRT1 |

Table 23 below shows the number of samples with Ct difference between multiplex and monoplex assays of more than 1.0 for shortlisted 3-plex combinations.

TABLE 23
Number of samples with Ct difference between multiplex and monoplex
assays of more than 1.0 for shortlisted 3-plex combinations

| | Gene 1 | CYSTM1/ | MCL1/ | FFAR2/ | S100A12/ | S100A12/ |
|---|---|---|---|---|---|---|
| | Gene 2 | SOD2/ | CYSTM1/ | SOD2/ | FFAR2/ | SOD2/ |
| Combination | Gene 3 | HPRT1 | HPRT1 | HPRT1 | HPRT1 | HPRT1 |
| In control | Gene 1 | 0 | 0 | 0 | 0 | 0 |
| samples | Gene 2 | 0 | 1 | 1 | 0 | 0 |
| | Gene 3 | 0 | 1 | 0 | 0 | 0 |
| In sepsis | Gene 1 | 0 | 0 | 0 | 0 | 0 |
| samples | Gene 2 | 0 | 0 | 0 | 0 | 0 |
| | Gene 3 | 6 | 2 | 0 | 5 | 5 |

FIG. 3 shows an unsupervised hierarchical clustering heatmap of the sepsis data panel (red=high expression, green=low expression). Row is gene, and column is sepsis/control sample. Highlighted samples are potential outliers.

### 6. Further Examples

To further demonstrate utilization of biomarker set or biomarker panel a subsequent cohort of 151 patients' samples was utilized. The sub-classification of the 151 samples is as follows: 36 controls, 6 SIRS without infection, 24 infection without SIRS, 67 mild Sepsis, 12 severe sepsis and 6 septic shock/cryptic shock. Examples in the following paragraphs are based on this sample set.

Table 24 below shows the predictive value (Area Under the Curve (AUC)) of each of the biomarkers of the biomarker panel of the 40 biomarkers or genes listed in List 1 for control versus sepsis. In some embodiments, the methods or kits respectively described herein use any one of the biomarkers or genes listed in Table 24.

TABLE 24
Predictive value (AUC) of each of the biomarkers (single
genes) of the biomarker panel for control versus sepsis,
with HPRT1 as the housekeeping gene.

| | |
|---|---|
| Up-regulated genes | Down-regulated genes |
| Area Under the Curve | Area Under the Curve |

| Genes | Area | Genes | Area |
|---|---|---|---|
| IL1RN | 0.903 | MAL | 0.887 |
| SLC22A4 | 0.820 | CCR7 | 0.828 |
| PLSCR1 | 0.916 | GZMK | 0.907 |
| ANXA3 | 0.887 | FCER1A | 0.870 |
| LRG1 | 0.877 | FAIM3 | 0.882 |
| C19ORF59 | 0.920 | CD3D | 0.923 |
| ACSL1 | 0.901 | CD6 | 0.830 |
| PFKFB3 | 0.870 | KLRB1 | 0.883 |
| FFAR2 | 0.874 | IL7R | 0.836 |
| FPR2 | 0.888 | CCL5 | 0.864 |
| HSPA1B | 0.905 | | |
| NT5C3 | 0.865 | | |
| DDX60L | 0.888 | | |
| SELL | 0.902 | | |
| IFITM1 | 0.902 | | |
| RAB24 | 0.885 | | |
| MCL1V1 | 0.862 | | |
| PROK2 | 0.862 | | |
| LILRA5 | 0.890 | | |
| TLR4 | 0.871 | | |
| NFIL3 | 0.903 | | |
| IL1B | 0.879 | | |
| CYSTM1 | 0.906 | | |
| CSF2RB | 0.865 | | |
| IFITM3 | 0.908 | | |
| SOD2 | 0.860 | | |
| FCGR1B | 0.906 | | |
| S100A12 | 0.908 | | |
| SP100 | 0.896 | | |
| NAIP | 0.897 | | |

In some embodiments, the methods or kits respectively described herein use one or more, and in any combination, of the 40 biomarkers or genes listed in List 1.

Table 25 below shows the predictive value (Area Under Curve (AUC)) of exemplary sets of two biomarkers of the biomarker panel of the 40 biomarkers or genes listed in. List 1 for control versus sepsis, with HPRT1/GAPDH as the housekeeping gene.

TABLE 25
Predictive value (AUC) of exemplary sets of two biomarkers or genes of the biomarker panel for control versus sepsis,
with HPRT1/GAPDH as the housekeeping gene.

| | HKG | HPRT1 | HPRT1 | HPRT1 | HPRT1 | HPRT1 | HPRT1 | HPRT1 | HPRT1 | HPRT1 | HPRT1 |
|---|---|---|---|---|---|---|---|---|---|---|---|
| HKG | Gene | IL1RN | SLC22A4 | PLSCR1 | ANXA3 | LRG1 | C19ORF59 | ACSL1 | PFKFB3 | FFAR2 | FPR2 |
| HPRT1 | IL1RN | — | 0.80 | 0.81 | 0.80 | 0.80 | 0.82 | 0.80 | 0.80 | 0.80 | 0.80 |
| HPRT1 | SLC22A4 | — | — | 0.81 | 0.79 | 0.78 | 0.80 | 0.79 | 0.78 | 0.78 | 0.78 |
| HPRT1 | PLSCR1 | — | — | — | 0.81 | 0.81 | 0.82 | 0.81 | 0.81 | 0.81 | 0.81 |
| HPRT1 | ANXA3 | — | — | — | — | 0.79 | 0.81 | 0.80 | 0.79 | 0.79 | 0.79 |
| HPRT1 | LRG1 | — | — | — | — | — | 0.81 | 0.79 | 0.78 | 0.78 | 0.79 |
| HPRT1 | C19ORF59 | — | — | — | — | — | — | 0.81 | 0.81 | 0.81 | 0.81 |
| HPRT1 | ACSL1 | — | — | — | — | — | — | — | 0.79 | 0.79 | 0.80 |
| HPRT1 | PFKFB3 | — | — | — | — | — | — | — | — | 0.78 | 0.79 |
| HPRT1 | FFAR2 | — | — | — | — | — | — | — | — | — | 0.79 |
| HPRT1 | FPR2 | — | — | — | — | — | — | — | — | — | — |
| HPRT1 | HSPA1B | — | — | — | — | — | — | — | — | — | — |
| HPRT1 | NT5C3 | — | — | — | — | — | — | — | — | — | — |
| HPRT1 | DDX60L | — | — | — | — | — | — | — | — | — | — |
| HPRT1 | SELL | — | — | — | — | — | — | — | — | — | — |
| HPRT1 | IFITM1 | — | — | — | — | — | — | — | — | — | — |
| HPRT1 | RAB24 | — | — | — | — | — | — | — | — | — | — |
| HPRT1 | MCL1 | — | — | — | — | — | — | — | — | — | — |
| HPRT1 | PROK2 | — | — | — | — | — | — | — | — | — | — |
| HPRT1 | LILRA5 | — | — | — | — | — | — | — | — | — | — |
| HPRT1 | TLR4 | — | — | — | — | — | — | — | — | — | — |
| | HKG | HPRT1 | HPRT1 | HPRT1 | HPRT1 | HPRT1 | HPRTI | HPRT1 | HPRT1 | HPRT1 | HPRT1 |
| HKG | Gene | HSPA1B | NT5C3 | DDX60L | SELL | IFITM1 | RAB24 | MCL1 | PROK2 | LILRA5 | TLR4 |
| HPRT1 | IL1RN | 0.82 | 0.79 | 0.80 | 0.81 | 0.81 | 0.80 | 0.80 | 0.80 | 0.80 | 0.80 |
| HPRT1 | SLC22A4 | 0.80 | 0.80 | 0.79 | 0.80 | 0.80 | 0.79 | 0.78 | 0.78 | 0.80 | 0.78 |
| HPRT1 | PLSCR1 | 0.83 | 0.80 | 0.81 | 0.82 | 0.81 | 0.81 | 0.81 | 0.81 | 0.81 | 0.81 |
| HPRT1 | ANXA3 | 0.81 | 0.80 | 0.80 | 0.81 | 0.81 | 0.80 | 0.80 | 0.79 | 0.80 | 0.80 |
| HPRT1 | LRG1 | 0.80 | 0.80 | 0.79 | 0.81 | 0.80 | 0.80 | 0.79 | 0.78 | 0.80 | 0.79 |
| HPRT1 | C19ORF59 | 0.83 | 0.82 | 0.81 | 0.82 | 0.82 | 0.82 | 0.81 | 0.81 | 0.82 | 0.81 |
| HPRT1 | ACSL1 | 0.81 | 0.80 | 0.80 | 0.81 | 0.81 | 0.80 | 0.80 | 0.79 | 0.81 | 0.80 |
| HPRT1 | PFKFB3 | 0.80 | 0.80 | 0.80 | 0.81 | 0.80 | 0.80 | 0.79 | 0.79 | 0.80 | 0.79 |
| HPRT1 | FFAR2 | 0.80 | 0.79 | 0.79 | 0.80 | 0.80 | 0.80 | 0.79 | 0.78 | 0.79 | 0.78 |
| HPRT1 | FPR2 | 0.81 | 0.80 | 0.80 | 0.81 | 0.80 | 0.80 | 0.79 | 0.79 | 0.80 | 0.79 |
| HPRT1 | HSPA1B | — | 0.82 | 0.82 | 0.82 | 0.82 | 0.81 | 0.81 | 0.81 | 0.82 | 0.81 |
| HPRT1 | NT5C3 | — | — | 0.79 | 0.81 | 0.80 | 0.80 | 0.80 | 0.80 | 0.80 | 0.80 |
| HPRT1 | DDX60L | — | — | — | 0.81 | 0.80 | 0.80 | 0.80 | 0.80 | 0.80 | 0.80 |
| HPRT1 | SELL | — | — | — | — | 0.82 | 0.81 | 0.81 | 0.81 | 0.81 | 0.81 |
| HPRT1 | IFITM1 | — | — | — | — | — | 0.81 | 0.80 | 0.80 | 0.81 | 0.80 |
| HPRT1 | RAB24 | — | — | — | — | — | — | 0.79 | 0.80 | 0.81 | 0.80 |
| HPRT1 | MCL1 | — | — | — | — | — | — | — | 0.79 | 0.80 | 0.79 |
| HPRT1 | PROK2 | — | — | — | — | — | — | — | — | 0.80 | 0.79 |
| HPRT1 | LILRA5 | — | — | — | — | — | — | — | — | — | 0.80 |
| HPRT1 | TLR4 | — | — | — | — | — | — | — | — | — | — |
| | HKG | HPRT1 | HPRT1 | HPRT1 | HPRT1 | HPRT1 | HPRT1 | HPRT1 | HPRT1 | HPRT1 | HPRT1 |
| HKG | Gene | NFIL3 | IL1B | CYSTM1 | CSF2RB | IFITM3 | SODZ | FCGR1B | S100A12 | SP100 | NAIP |
| HPRT1 | IL1RN | 0.81 | 0.80 | 0.81 | 0.80 | 0.81 | 0.80 | 0.81 | 0.82 | 0.80 | 0.81 |
| HPRT1 | SLC22A4 | 0.80 | 0.79 | 0.81 | 0.78 | 0.80 | 0.79 | 0.79 | 0.80 | 0.80 | 0.79 |
| HPRT1 | PLSCR1 | 0.82 | 0.81 | 0.82 | 0.81 | 0.81 | 0.81 | 0.81 | 0.83 | 0.81 | 0.82 |
| HPRT1 | ANXA3 | 0.81 | 0.80 | 0.81 | 0.79 | 0.81 | 0.80 | 0.80 | 0.81 | 0.80 | 0.81 |
| HPRT1 | LRG1 | 0.80 | 0.79 | 0.81 | 0.79 | 0.80 | 0.79 | 0.80 | 0.81 | 0.80 | 0.80 |
| HPRT1 | C19ORF59 | 0.82 | 0.81 | 0.83 | 0.81 | 0.82 | 0.81 | 0.82 | 0.83 | 0.82 | 0.82 |
| HPRT1 | ACSL1 | 0.81 | 0.80 | 0.81 | 0.80 | 0.81 | 0.80 | 0.81 | 0.81 | 0.80 | 0.81 |
| HPRT1 | PFKFB3 | 0.80 | 0.79 | 0.81 | 0.78 | 0.80 | 0.79 | 0.80 | 0.81 | 0.80 | 0.80 |
| HPRT1 | FFAR2 | 0.80 | 0.79 | 0.80 | 0.78 | 0.80 | 0.79 | 0.79 | 0.80 | 0.80 | 0.80 |
| HPRT1 | FPR2 | 0.81 | 0.80 | 0.81 | 0.79 | 0.81 | 0.80 | 0.80 | 0.81 | 0.80 | 0.80 |
| HPRT1 | HSPA1B | 0.81 | 0.81 | 0.82 | 0.81 | 0.82 | 0.81 | 0.82 | 0.83 | 0.82 | 0.82 |
| HPRT1 | NT5C3 | 0.80 | 0.80 | 0.81 | 0.80 | 0.80 | 0.80 | 0.80 | 0.81 | 0.79 | 0.82 |
| HPRT1 | DDX60L | 0.81 | 0.80 | 0.81 | 0.80 | 0.81 | 0.80 | 0.80 | 0.81 | 0.80 | 0.81 |
| HPRT1 | SELL | 0.82. | 0.81 | 0.82 | 0.81 | 0.82 | 0.81 | 0.81 | 0.82 | 0.81 | 0.82 |
| HPRT1 | IFITM1 | 0.81 | 0.81 | 0.82 | 0.80 | 0.81 | 0.80 | 0.81 | 0.82 | 0.80 | 0.82 |
| HPRT1 | RAB24 | 0.81 | 0.80 | 0.82 | 0.80 | 0.81 | 0.80 | 0.81 | 0.81 | 0.81 | 0.81 |
| HPRT1 | MCL1 | 0.80 | 0.79 | 0.81 | 0.79 | 0.81 | 0.80 | 0.80 | 0.82 | 0.80 | 0.80 |
| HPRT1 | PROK2 | 0.80 | 0.79 | 0.81 | 0.79 | 0.81 | 0.80 | 0.80 | 0.81 | 0.80 | 0.80 |
| HPRT1 | LILRA5 | 0.81 | 0.81 | 0.82 | 0.80 | 0.81 | 0.80 | 0.80 | 0.81 | 0.81 | 0.81 |
| HPRT1 | TLR4 | 0.80 | 0.79 | 0.81 | 0.79 | 0.80 | 0.79 | 0.80 | 0.81 | 0.80 | 0.80 |
| | HKG | HPRT1 | HPRT1 | HPRT1 | HPRT1 | HPRT1 | HPRT1 | HPRT1 | HPRT1 | HPRT1 | HPRT1 |
| HKG | Gene | MAL1 | CCR7 | GZMK | FCER1A | FAIM3 | CD3D | CD6 | KLRB1 | IL7R | CCL5 |
| HPRT1 | IL1RN | 0.81 | 0.79 | 0.82 | 0.80 | 0.81 | 0.82 | 0.80 | 0.82 | 0.81 | 0.82 |
| HPRT1 | SLC22A4 | 0.81 | 0.79 | 0.80 | 0.80 | 0.82 | 0.82 | 0.80 | 0.82 | 0.80 | 0.82 |
| HPRT1 | PLSCR1 | 0.81 | 0.80 | 0.83 | 0.81 | 0.82 | 0.83 | 0.81 | 0.82 | 0.81 | 0.82 |
| HPRT1 | ANXA3 | 0.81 | 0.80 | 0.81 | 0.80 | 0.82 | 0.83 | 0.81 | 0.82 | 0.81 | 0.82 |
| HPRT1 | LRG1 | 0.82 | 0.80 | 0.81 | 0.80 | 0.81 | 0.82 | 0.81 | 0.82 | 0.81 | 0.82 |
| HPRT1 | C19ORF59 | 0.83 | 0.81 | 0.82 | 0.81 | 0.82 | 0.83 | 0.82 | 0.83 | 0.82 | 0.83 |
| HPRT1 | ACSL1 | 0.82 | 0.81 | 0.81 | 0.81 | 0.82 | 0.83 | 0.81 | 0.82 | 0.81 | 0.82 |
| HPRT1 | PFKFB3 | 0.82 | 0.80 | 0.81 | 0.81 | 0.82 | 0.82 | 0.80 | 0.82 | 0.81 | 0.82 |
| HPRT1 | FFAR2 | 0.81 | 0.79 | 0.81 | 0.80 | 0.81 | 0.82 | 0.81 | 0.82 | 0.80 | 0.82 |
| HPRT1 | FPR2 | 0.82 | 0.80 | 0.81 | 0.80 | 0.82 | 0.82 | 0.81 | 0.82 | 0.81 | 0.82 |
| HPRT1 | HSPA1B | 0.84 | 0.83 | 0.83 | 0.83 | 0.84 | 0.84 | 0.83 | 0.84 | 0.83 | 0.84 |
| HPRT1 | NT5C3 | 0.80 | 0.79 | 0.82 | 0.80 | 0.80 | 0.82 | 0.78 | 0.81 | 0.79 | 0.81 |
| HPRT1 | DDX60L | 0.81 | 0.80 | 0.82 | 0.80 | 0.81 | 0.82 | 0.80 | 0.82 | 0.80 | 0.82 |
| HPRT1 | SELL | 0.82 | 0.81 | 0.83 | 0.81 | 0.83 | 0.83 | 0.82 | 0.83 | 0.82 | 0.83 |
| HPRT1 | IFITM1 | 0.81 | 0.80 | 0.82 | 0.80 | 0.81 | 0.83 | 0.81 | 0.82 | 0.81 | 0.82 |
| HPRT1 | RAB24 | 0.82 | 0.80 | 0.82 | 0.81 | 0.82 | 0.82 | 0.81 | 0.82 | 0.81 | 0.82 |
| HPRT1 | MCL1 | 0.82 | 0.80 | 0.82 | 0.81 | 0.82 | 0.82 | 0.81 | 0.83 | 0.81 | 0.82 |
| HPRT1 | PROK2 | 0.81 | 0.80 | 0.81 | 0.80 | 0.81 | 0.82 | 0.80 | 0.82 | 0.80 | 0.82 |
| HPRT1 | LILRA5 | 0.81 | 0.80 | 0.81 | 0.80 | 0.81 | 0.82 | 0.81 | 0.82 | 0.81 | 0.82 |
| HPRT1 | TLR4 | 0.82 | 0.80 | 0.81 | 0.80 | 0.81 | 0.82 | 0.81 | 0.82 | 0.81 | 0.82 |
| | HKG | HPRT1 | HPRT1 | HPRT1 | HPRT1 | HPRT1 | HPRT1 | HPRT1 | HPRT1 | HPRT1 | HPRT1 |
| HKG | Gene | NFIL3 | IL1B | CYSTM1 | CSF2RB | IFITM3 | SOD2 | FCGR1B | S100A12 | SP100 | NAIP |
| HPRT1 | NFIL3 | — | 0.81 | 0.82 | 0.80 | 0.81 | 0.80 | 0.81 | 0.82 | 0.81 | 0.81 |
| HPRT1 | IL1B | — | — | 0.81 | 0.79 | 0.81 | 0.79 | 0.80 | 0.81 | 0.80 | 0.80 |
| HPRT1 | CYSTM1 | — | — | — | 0.81 | 0.82 | 0.81 | 0.82 | 0.82 | 0.81 | 0.82 |
| HPRT1 | CSF2RB | — | — | — | — | 0.80 | 0.79 | 0.80 | 0.80 | 0.80 | 0.80 |
| HPRT1 | IFITM3 | — | — | — | — | — | 0.81 | 0.81 | 0.82 | 0.81 | 0.82 |
| HPRT1 | SOD2 | — | — | — | — | — | — | 0.80 | 0.81 | 0.80 | 0.81 |
| HPRT1 | FCGR1B | — | — | — | — | — | — | — | 0.82 | 0.80 | 0.81 |
| HPRT1 | S100A12 | — | — | — | — | — | — | — | — | 0.82 | 0.82 |
| HPRT1 | SP100 | — | — | — | — | — | — | — | — | — | 0.81 |
| HPRT1 | NAIP | — | — | — | — | — | — | — | — | — | — |
| HPRT1 | MAL1 | — | — | — | — | — | — | — | — | — | — |
| HPRT1 | CCR7 | — | — | — | — | — | — | — | — | — | — |
| HPRT1 | GZMK | — | — | — | — | — | — | — | — | — | — |
| HPRT1 | FCER1A | — | — | — | — | — | — | — | — | — | — |
| HPRT1 | FAIM3 | — | — | — | — | — | — | — | — | — | — |
| HPRT1 | CD3D | — | — | — | — | — | — | — | — | — | — |
| HPRT1 | CD6 | — | — | — | — | — | — | — | — | — | — |
| HPRT1 | KLRB1 | — | — | — | — | — | — | — | — | — | — |
| HPRT1 | IL7R | — | — | — | — | — | — | — | — | — | — |
| HPRT1 | CCL5 | — | — | — | — | — | — | — | — | — | — |
| | HKG | HPRT1 | HPRT1 | HPRT1 | HPRT1 | HPRT1 | HPRT1 | HPRT1 | HPRT1 | HPRT1 | HPRT1 |
| HKG | Gene | MAL1 | CCR7 | GZMK | FCER1A | FAIM3 | CD3D | CD6 | KLRB1 | IL7R | CCLS |
| HPRT1 | NFIL3 | 0.82 | 0.80 | 0.82 | 0.81 | 0.82 | 0.83 | 0.81 | 0.83 | 0.81 | 0.82 |
| HPRT1 | IL1B | 0.81 | 0.79 | 0.81 | 0.80 | 0.81 | 0.82 | 0.80 | 0.82 | 0.80 | 0.82 |
| HPRT1 | CYSTM1 | 0.83 | 0.81 | 0.82 | 0.82 | 0.83 | 0.83 | 0.82 | 0.83 | 0.82 | 0.83 |
| HPRT1 | CSF2RB | 0.81 | 0.80 | 0.81 | 0.80 | 0.82 | 0.82 | 0.81 | 0.82 | 0.81 | 0.82 |
| HPRT1 | IFITM3 | 0.81 | 0.80 | 0.82 | 0.81 | 0.82 | 0.83 | 0.81 | 0.83 | 0.81 | 0.83 |
| HPRT1 | SOD2 | 0.82 | 0.80 | 0.81 | 0.80 | 0.82 | 0.82 | 0.80 | 0.82 | 0.81 | 0.82 |
| HPRT1 | FCGR1B | 0.81 | 0.80 | 0.82 | 0.80 | 0.82 | 0.83 | 0.81 | 0.82 | 0.81 | 0.82 |
| HPRT1 | S100A12 | 0.83 | 0.81 | 0.82 | 0.81 | 0.82 | 0.83 | 0.81 | 0.83 | 0.82 | 0.83 |
| HPRT1 | SP100 | 0.81 | 0.80 | 0.82 | 0.81 | 0.81 | 0.82 | 0.80 | 0.83 | 0.80 | 0.82 |
| HPRT1 | NAIP | 0.82 | 0.81 | 0.82 | 0.81 | 0.82 | 0.83 | 0.81 | 0.83 | 0.81 | 0.82 |
| HPRT1 | MAL1 | — | 0.77 | 0.82 | 0.79 | 0.80 | 0.82 | 0.79 | 0.81 | 0.78 | 0.81 |
| HPRT1 | CCR7 | — | — | 0.80 | 0.78 | 0.78 | 0.80 | 0.76 | 0.79 | 0.76 | 0.79 |
| HPRT1 | GZMK | — | — | — | 0.83 | 0.82 | 0.82 | 0.80 | 0.82 | 0.80 | 0.81 |
| HPRT1 | FCER1A | — | — | — | — | 0.80 | 0.82 | 0.79 | 0.80 | 0.79 | 0.80 |
| HPRT1 | FAIM3 | — | — | — | — | — | 0.82 | 0.78 | 0.80 | 0.79 | 0.80 |
| HPRT1 | CD3D | — | — | — | — | — | — | 0.80 | 0.82 | 0.80 | 0.81 |
| HPRT1 | CD6 | — | — | — | — | — | — | — | 0.79 | 0.77 | 0.78 |
| HPRT1 | KLRB1 | — | — | — | — | — | — | — | — | 0.80 | 0.81 |
| HPRT1 | IL7R | — | — | — | — | — | — | — | — | — | 0.79 |
| HPRT1 | CCL5 | — | — | — | — | — | — | — | — | — | — |
| | HKG | GAPDH | GAPDH | GAPDH | GAPDH | GAPDH | GAPDH | GAPDH | GAPDH | GAPDH | GAPDH |
| HKG | Gene | IL1RN | SLC22A4 | PLSCR1 | ANXA3 | LRG1 | C19ORF59 | ACSL1 | PFKFB3 | FFAR2 | FPR2 |
| HPRT1 | IL1RN | 0.81 | 0.82 | 0.82 | 0.84 | 0.83 | 0.86 | 0.84 | 0.83 | 0.82 | 0.82 |
| HPRT1 | SLC22A4 | 0.82 | 0.77 | 0.83 | 0.82 | 0.82 | 0.85 | 0.83 | 0.81 | 0.81 | 0.81 |
| HPRT1 | PLSCR1 | 0.82 | 0.82 | 0.82 | 0.85 | 0.84 | 0.86 | 0.84 | 0.84 | 0.83 | 0.83 |
| HPRT1 | ANXA3 | 0.82 | 0.80 | 0.82 | 0.82 | 0.83 | 0.85 | 0.83 | 0.82 | 0.81 | 0.82 |
| HPRT1 | LRG1 | 0.82 | 0.79 | 0.83 | 0.83 | 0.83 | 0.85 | 0.83 | 0.81 | 0.81 | 0.82 |
| HPRT1 | C19ORF59 | 0.83 | 0.82 | 0.84 | 0.84 | 0.84 | 0.86 | 0.85 | 0.83 | 0.83 | 0.83 |
| HPRT1 | ACSL1 | 0.82 | 0.81 | 0.83 | 0.83 | 0.83 | 0.86 | 0.84 | 0.82 | 0.82 | 0.82 |
| HPRT1 | PFKFB3 | 0.82 | 0.79 | 0.83 | 0.83 | 0.82 | 0.85 | 0.83 | 0.81 | 0.81 | 0.82 |
| HPRT1 | FFAR2 | 0.81 | 0.79 | 0.82 | 0.83 | 0.82 | 0.85 | 0.83 | 0.82 | 0.80 | 0.81 |
| HPRT1 | FPR2 | 0.82 | 0.80 | 0.83 | 0.83 | 0.83 | 0.86 | 0.84 | 0.82 | 0.81 | 0.82 |
| HPRT1 | HSPA1B | 0.84 | 0.81 | 0.84 | 0.85 | 0.84 | 0.87 | 0.85 | 0.83 | 0.83 | 0.83 |
| HPRT1 | NT5C3 | 0.80 | 0.80 | 0.80 | 0.83 | 0.83 | 0.86 | 0.83 | 0.82 | 0.81 | 0.81 |
| HPRT1 | DDX60L | 0.81 | 0.80 | 0.82 | 0.83 | 0.83 | 0.86 | 0.83 | 0.82 | 0.81 | 0.82 |
| HPRT1 | SELL | 0.82 | 0.81 | 0.83 | 0.84 | 0.84 | 0.86 | 0.84 | 0.83 | 0.82 | 0.82 |
| HPRT1 | IFITM1 | 0.82 | 0.81 | 0.82 | 0.84 | 0.83 | 0.86 | 0.84 | 0.83 | 0.82 | 0.82 |
| HPRT1 | RAB24 | 0.82 | 0.80 | 0.82 | 0.83 | 0.83 | 0.85 | 0.83 | 0.82 | 0.82 | '0.82 |
| HPRT1 | MCL1 | 0.82 | 0.79 | 0.82 | 0.83.. | 0.82 | 0.85 | 0.83 | 0.82 | 0.81 | 0.82 |
| HPRT1 | PROK2 | 0.82 | 0.80 | 0.83 | 0.83 | 0.83 | 0.85 | 0.83 | 0.81 | 0.81 | 0.82 |
| HPRT1 | LILRA5 | 0.82 | 0.81 | 0.82 | 0.83 | 0.84 | 0.85 | 0.84 | 0.82 | 0.82 | 0.82 |
| HPRT1 | TLR4 | 0.81 | 0.79 | 0.83 | 0.83 | 0.83 | 0.85 | 0.84 | 0.81 | 0.81 | 0.82 |
| | HKG | GAPDH | GAPDH | GAPDH | GAPDH | GAPDH | GAPDH | GAPDH | GAPDH | GAPDH | GAPDH |
| HKG | Gene | HSPA1B | NT5C3 | DDX60L | SELL | IFITM1 | RAB24 | MCL1 | PROK2 | LILRA5 | TLR4 |
| HPRT1 | IL1RN | 0.81 | 0.79 | 0.81 | 0.84 | 0.82 | 0.80 | 0.81 | 0.84 | 0.81 | 0.82 |
| HPRT1 | SLC22A4 | 0.78 | 0.79 | 0.80 | 0.83 | 0.83 | 0.79 | 0.79 | 0.82 | 0.82 | 0.81 |
| HPRT1 | PLSCR1 | 0.81 | 0.79 | 0.81 | 0.84 | 0.82 | 0.80 | 0.81 | 0.85 | 0.81 | 0.83 |
| HPRT1 | ANXA3 | 0.80 | 0.81 | 0.81 | 0.84 | 0.83 | 0.80 | 0.81 | 0.83 | 0.82 | 0.82 |
| HPRT1 | LRG1 | 0.80 | 0.81 | 0.83. | 0.84 | 0.83 | 0.80 | 0.80 | 0.83 | 0.82 | 0.81 |
| HPRT1 | C19ORF59 | 0.82 | 0.82 | 0.83 | 0.85 | 0.85 | 0.81 | 0.82 | 0.84 | 0.83 | 0.84 |
| HPRT1 | ACSL1 | 0.80 | 0.81 | 0.81 | 0.84 | 0.84 | 0.80 | 0.81 | 0.83 | 0.82 | 0.82 |
| HPRT1 | PFKFB3 | 0.79 | 0.80 | 0.81 | 0.84 | 0.83 | 0.79 | 0.80 | 0.82 | 0.81 | 0.82 |
| HPRT1 | FFAR2 | 0.79 | 0.79 | 0.80 | 0.83 | 0.82 | 0.79 | 0.79 | 0.83 | 0.81 | 0.81 |
| HPRT1 | FPR2 | 0.80 | 0.80 | 0.81 | 0.84 | 0.83 | 0.80 | 0.80 | 0.83 | 0.81 | 0.82 |
| HPRT1 | HSPA1B | 0.80 | 0.83 | 0.83 | 0.85 | 0.85 | 0.81 | 0.82 | 0.84 | 0.84 | 0.83 |
| HPRT1 | NT5C3 | 0.80 | 0.75 | 0.79 | 0.82 | 0.80 | 0.78 | 0.79 | 0.84 | 0.81 | 0.81 |
| HPRT1 | DDX60L | 0.80 | 0.79 | 0.80 | 0.83 | 0.82 | 0.79 | 0.80 | 0.84 | 0.81 | 0.81 |
| HPRT1 | SELL | 0.81 | 0.81 | 0.82 | 0.84 | 0.84 | 0.81 | 0.81 | 0.84 | 0.82 | 0.83 |
| HPRT1 | IFITM1 | 0.81 | 0.79 | 0.80 | 0.83 | 0.82 | 0.80 | 0.81 | 0.84 | 0.81 | 0.82 |
| HPRT1 | RAB24 | 0.80 | 0.80 | 0.81 | 0.84 | 0.83 | 0.80 | 0.80 | 0.83 | 0.82 | 0.81 |
| HPRT1 | MCL1 | 0.80 | d.80 | 0.80 | 0.84 | 0.83 | 0.79 | 0.79 | 0.83 | 0.81 | 0.81 |
| HPRT1 | PROK2 | 0.79 | 0.81 | 0.81 | 0.84 | 0.83 | 0.80 | 0.80 | 0.82 | 0.82 | 0.82 |
| HPRT1 | LILRA5 | 0.80 | 0.80 | 0.81 | 0.84 | 0.83 | 0.80 | 0.81 | 0.84 | 0.81 | 0.82 |
| HPRT1 | TLR4 | 0.79 | 0.80 | 0.81 | 0.84 | 0.83 | 0.79 | 0.80 | 0.83 | 0.82 | 0.81 |
| | HKG | GAPDH | GAPDH | GAPDH | GAPDH | GAPDH | GAPDH | GAPDH | GAPDH | GAPDH | GAPDH |
| HKG | Gene | IL1RN | SLC22A4 | PLSCR1 | ANXA3 | LRG1 | C19ORF59 | ACSL1 | PFKFB3 | FFAR2 | FPR2 |
| HPRT1 | NFIL3 | 0.83 | 0.81 | 0.83 | 0.84 | 0.84 | 0.86 | 0.84 | 0.83 | 0.82 | 0.83 |
| HPRT1 | IL1B | 0.81 | 0.80 | 0.82 | 0.83 | 0.83 | 0.85 | 0.83 | 0.82 | 0.82 | 0.82 |
| HPRT1 | CYSTM1 | 0.83 | 0.82 | 0.84 | 0.84 | 0.84 | 0.86 | 0.84 | 0.83 | 0.83 | 0.83 |
| HPRT1 | CSF2RB | 0.82 | 0.79 | 0.83 | 0.83 | 0.82 | 0.85 | 0.83 | 0.81 | 0.81 | 0.82 |
| HPRT1 | IFITM3 | 0.82 | 0.82 | 0.82 | 0.84 | 0.83 | 0.86 | 0.84 | 0.83 | 0.82 | 0.83 |
| HPRT1 | SOD2 | 0.82 | 0.79 | 0.83 | 0.83 | 0.83 | 0.85 | 0.83 | 0.82 | 0.81 | 0.81 |
| HPRTI | FCGR1B | 0.82 | 0.80 | 0.82 | 0.84 | 0.83 | 0.86 | 0.84 | 0.83 | 0.81 | 0.82 |
| HPRT1 | S100A12 | 0.84 | 0.82 | 0.84 | 0.84 | 0.84 | 0.86 | 0.85 | 0.83 | 0.83 | 0.83 |
| HPRT1 | SP100 | 0.81 | 0.80 | 0.82 | 0.83 | 0.82 | 0.85 | 0.84 | 0.82 | 0.81 | 0.82 |
| HPRT1 | NAIP | 0.83 | 0.81 | 0.84 | 0.84 | 0.84 | 0.86 | 0.84 | 0.83 | 0.82 | 0.83 |
| HPRT1 | MAL1 | 0.82 | 0.81 | 0.81 | 0.84 | 0.85 | 0.85 | 0.84 | 0.83 | 0.83 | 0.83 |
| HPRT1 | CCR7 | 0.81 | 0.79 | 0.80 | 0.83 | 0.84 | 0.84 | 0.84 | 0.82 | 0.81 | 0.81 |
| HPRT1 | GZMK | 0.83 | 0.82 | 0.83 | 0.84 | 0.84 | 0.86 | 0.85 | 0.84 | 0.83 | 0.83 |
| HPRT1 | FCER1A | 0.82 | 0.81 | 0.81 | 0.83 | 0.84 | 0.85 | 0.84 | 0.83 | 0.83 | 0.82 |
| HPRT1 | FAIM3 | 0.82 | 0.82 | 0.82 | 0.84 | 0.85 | 0.85 | 0.85 | 0.83 | 0.83 | 0.83 |
| HPRT1 | CD3D | 0.83 | 0.83 | 0.83 | 0.85 | 0.85 | 0.86 | 0.85 | 0.84 | 0.84 | 0.84 |
| HPRT1 | CD6 | 0.81 | 0.79 | 0.80 | 0.83 | 0.84 | 0.85 | 0.84 | 0.82 | 0.82 | 0.82 |
| HPRT1 | KLRB1 | 0.83 | 0.84 | 0.83 | 0.85 | 0.85 | 0.86 | 0.86 | 0.84 | 0.84 | 0.84 |
| HPRT1 | IL7R | 0.81 | 0.79 | 0.80 | 0.83 | 0.84 | 0.85 | 0.84 | 0.82 | 0.82 | 0.82 |
| HPRT1 | CCL5 | 0.83 | 0.82 | 0.83 | 0.84 | 0.85 | 0.86 | 0.85 | 0.83 | 0.84 | 0.83 |
| | HKG | GAPDH | GAPDH | GAPDH | GAPDH | GAPDH | GAPDH | GAPDH | GAPDH | GAPDH | GAPDH |
| HKG | Gene | HSPA1B | NT5C3 | DDX60L | SELL | IFITM1 | RAB24 | MCL1 | PROK2 | LILRA5 | TLR4 |
| HPRT1 | NFIL3 | 0.80 | 0.81 | 0.81 | 0.84 | 0.83 | 0.80 | 0.81 | 0.84 | 0.82 | 0.82 |
| HPRT1 | IL1B | 0.80 | 0.80 | 0.81 | 0.84 | 0.83 | 0.79 | 0.80 | 0.83 | 0.81 | 0.82 |
| HPRT1 | CYSTM1 | 0.81 | 0.82 | 0.82 | 0.85 | 0.84 | 0.81 | 0.82 | 0.84 | 0.83 | 0.83 |
| HPRT1 | CSF2RB | 0.79 | 0.80 | 0.81 | 0.83 | 0.83 | 0.79 | 0.79 | 0.83 | 0.81 | 0.81 |
| HPRT1 | IFITM3 | 0.81 | 0.80 | 0.81 | 0.83 | 0.82 | 0.80 | 0.81 | 0.84 | 0.82 | 0.82 |
| HPRT1 | SOD2 | 0.79 | 0.80 | 0.81 | 0.83 | 0.83 | 0.79 | 0.80 | 0.83 | 0.82 | 0.81 |
| HPRTI | FCGR1B | 0.81 | 0.79 | 0.80 | 0.83 | 0.82 | 0.80 | 0.80 | 0.84 | 0.81 | 0.82 |
| HPRT1 | S100A12 | 0.81 | 0.82 | 0.83 | 0.85 | 0.84 | 0.81 | 0.82 | 0.84 | 0.83 | 0.83 |
| HPRT1 | SP100 | 0.79 | 0.78 | 0.80 | 0.83 | 0.82 | 0.79 | 0.80 | 0.83 | 0.81 | 0.81 |
| HPRT1 | NAIP | 0.81 | 0.81 | 0.82 | 0.84 | 0.84 | 0.81 | 0.81 | 0.83 | 0.82 | 0.83 |
| HPRT1 | MAL1 | 0.81 | 0.78 | 0.81 | 0.83 | 0.81 | 0.80 | 0.80 | 0.84 | 0.82 | 0.82 |
| HPRT1 | CCR7 | 0.79 | 0.75 | 0.79 | 0.83 | 0.80 | 0.78 | 0.79 | 0.83 | 0.80 | 0.81. |
| HPRT1 | GZMK | 0.82 | 0.81 | 0.83 | 0.85 | 0.84 | 0.81 | 0.82 | 0.85 | 0.83 | 0.83 |
| HPRT1 | FCER1A | 0.81 | 0.78 | 0.81 | 0.84 | 0.82 | 0.79 | 0.82 | 0.84 | 0.81 | 0.83 |
| HPRT1 | FAIM3 | 0.81 | 0.79 | 0.82 | 0.84 | 0.82 | 0.80 | 0.81 | 0.85 | 0.81 | 0.83 |
| HPRT1 | CD3D | 0.82 | 0.81 | 0.83 | 0.85 | 0.84 | 0.82 | 0.83 | 0.85 | 0.82 | 0.84 |
| HPRT1 | CD6 | 0.79 | 0.76 | 0.80 | 0.83 | 0.80 | 0.77 | 0.79 | 0.83 | 0.80 | 0.81 |
| HPRT1 | KLRB1 | 0.82 | 0.80 | 0.83 | 0.85 | 0.84 | 0.82 | 0.83 | 0.86 | 0.83 | 0.84 |
| HPRT1 | IL7R | 0.79 | 0.76 | 0.80 | 0.83 | 0.80 | 0.78 | 0.79 | 0.83 | 0.80 | 0.82 |
| HPRT1 | CCL5 | 0.81 | 0.79 | 0.82 | 0.84 | 0.83 | 0.80 | 0.82 | 0.85 | 0.82 | 0.83 |
| | HKG | GAPDH | GAPDH | GAPDH | GAPDH | GAPDH | GAPDH | GAPDH | GAPDH | GAPDH | GAPDH |
| HKG | Gene | IL1RN | SLC22A4 | PLSCR1 | ANXA3 | LRG1 | C19ORF59 | ACSL1 | PFKFB3 | FFAR2 | FPR2 |
| GAPDH | IL1RN | — | 0.82 | 0.82 | 0.85 | 0.85 | 0.87 | 0.85 | 0.84 | 0.82 | 0.83 |
| GAPDH | SLC22A4 | — | — | 0.81 | 0.81 | 0.82 | 0.85 | 0.83 | 0.80 | 0.79 | 0.80 |
| GAPDH | PLSCR1 | — | — | — | 0.85 | 0.85 | 0.87 | 0.85 | 0.84 | 0.83 | 0.83 |
| GAPDH | ANXA3 | — | — | — | — | 0.84 | 0.86 | 0.85 | 0.83 | 0.84 | 0.84 |
| GAPDH | LRG1 | — | — | — | — | — | 0.87 | 0.85 | 0.83 | 0.83 | 0.84 |
| GAPDH | C19ORF59 | — | — | — | — | — | — | 0.87 | 0.85 | 0.86 | 0.86 |
| GAPDH | ACSL1 | — | — | — | — | — | — | — | 0.84 | 0.84 | 0.84 |
| GAPDH | PFKFB3 | — | — | — | — | — | — | — | — | 0.83 | 0.83 |
| GAPDH | FFAR2 | — | — | — | — | — | — | — | — | — | 0.82 |
| GAPDH | FPR2 | — | — | — | — | — | — | — | — | — | — |
| GAPDH | HSPA1B | — | — | — | — | — | — | — | — | — | — |
| GAPDH | NT5C3 | — | — | — | — | — | — | — | — | — | — |
| GAPDH | DDX60L | — | — | — | — | — | — | — | — | — | — |
| GAPDH | SELL | — | — | — | — | — | — | — | — | — | — |
| GAPDH | IFITM1 | — | — | — | — | — | — | — | — | — | — |
| GAPDH | RAB24 | — | — | — | — | — | — | — | — | — | — |
| GAPDH | MCL1 | — | — | — | — | — | — | — | — | — | — |
| GAPDH | PROK2 | — | — | — | — | — | — | — | — | — | — |
| GAPDH | LILRA5 | — | — | — | — | — | — | — | — | — | — |
| GAPDH | TLR4 | — | — | — | — | — | — | — | — | — | — |
| | HKG | GAPDH | GAPDH | GAPDH | GAPDH | GAPDH | GAPDH | GAPDH | GAPDH | GAPDH | GAPDH |
| HKG | Gene | HSPA1B | NT5C3 | DDX60L | SELL | IFITM1 | RAB24 | MCL1 | PROK2 | LILRA5 | TLR4 |
| GAPDH | IL1RN | 0.81 | 0.80 | 0.81 | 0.84 | 0.82 | 0.80 | 0.81 | 0.85 | 0.82 | 0.83 |
| GAPDH | SLC22A4 | 0.77 | 0.74 | 0.78 | 0.82 | 0.81 | 0.76 | 0.76 | 0.80 | 0.80 | 0.80 |
| GAPDH | PLSCR1 | 0.81 | 0.78 | 0.80 | 0.84 | 0.81 | 0.80 | 0.81 | 0.86 | 0.82 | 0.83 |
| GAPDH | ANXA3 | 0.83 | 0.83 | 0.83 | 0.85 | 0.85 | 0.82 | 0.83 | 0.84 | 0.84 | 0.84 |
| GAPDH | LRG1 | 0.83 | 0.84 | 0.83 | 0.85 | 0.85 | 0.82 | 0.83 | 0.84 | 0.84 | 0.84 |
| GAPDH | C19ORF59 | 0.85 | 0.86 | 0.87 | 0.87 | 0.87 | 0.84 | 0.86 | 0.86 | 0.86 | 0.86 |
| GAPDH | ACSL1 | 0.83 | 0.83 | 0.83 | 0.85 | 0.85 | 0.82 | 0.83 | 0.84 | 0.84 | 0.84 |
| GAPDH | PFKFB3 | 0.81 | 0.81 | 0.81 | 0.85 | 0.84 | 0.80 | 0.81 | 0.82 | 0.83 | 0.82 |
| GAPDH | FFAR2 | 0.81 | 0.79 | 0.80 | 0.84 | 0.82 | 0.80 | 0.80 | 0.83 | 0.82 | 0.82 |
| GAPDH | FPR2 | 0.81 | 0.80 | 0.81 | 0.84 | 0.83 | 0.80 | 0.81 | 0.84 | 0.82 | 0.83 |
| GAPDH | HSPA1B | — | 0.76 | 0.79 | 0.82 | 0.82 | 0.76 | 0.78 | 0.83 | 0.81 | 0.81 |
| GAPDH | NT5C3 | — | — | 0.75 | 0.80 | 0.78 | 0.74 | 0.74 | 0.83 | 0.79 | 0.80 |
| GAPDH | DDX60L | — | — | — | 0.82 | 0.80 | 0.78 | 0.77 | 0.83 | 0.80 | 0.81 |
| GAPDH | SELL | — | — | — | — | 0.83 | 0.81 | 0.81 | 0.85 | 0.83 | 0.84 |
| GAPDH | IFITM1 | — | — | — | — | — | 0.80 | 0.79 | 0.85 | 0.82 | 0.83 |
| GAPDH | RAB24 | — | — | — | — | — | — | 0.76 | 0.81 | 0.79 | 0.79 |
| GAPDH | MCL1 | — | — | — | — | — | — | — | 0.82 | 0.80 | 0.80 |
| GAPDH | PROK2 | — | — | — | — | — | — | — | — | 0.84 | 0.83 |
| GAPDH | LILRA5 | — | — | — | — | — | — | — | — | — | 0.83 |
| GAPDH | TLR4 | — | — | — | — | — | — | — | — | — | — |
| | HKG | GAPDH | GAPDH | GAPDH | GAPDH | GAPDH | GAPDH | GAPDH | GAPDH | GAPDH | GAPDH |
| HKG | Gene | NFIL3 | IL1B | CYSTM1 | CSF2RB | IFITM3 | SOD2 | FCGR1B | S100A12 | SP100 | NAIP |
| GAPDH | IL1RN | 0.83 | 0.82 | 0.86 | 0.83 | 0.82 | 0.83 | 0.82 | 0.86 | 0.79 | 0.84 |
| GAPDH | SLC22A4 | 0.80 | 0.80 | 0.83 | 0.79 | 0.80 | 0.79 | 0.80 | 0.85 | 0.74 | 0.80 |
| GAPDH | PLSCR1 | 0.83 | 0.83 | 0.85 | 0.83 | 0.81 | 0.83 | 0.81 | 0.87 | 0.78 | 0.84 |
| GAPDH | ANXA3 | 0.85 | 0.84 | 0.85 | 0.83 | 0.85 | 0.83 | 0.84 | 0.86 | 0.82 | 0.85 |
| GAPDH | LRG1 | 0.85 | 0.84 | 0.85 | 0.82 | 0.85 | 0.83 | 0.84 | 0.87 | 0.83 | 0.84 |
| GAPDH | C19ORF59 | 0.87 | 0.86 | 0.87 | 0.85 | 0.87 | 0.85 | 0.87 | 0.87 | 0.85 | 0.86 |
| GAPDH | ACSL1 | 0.86 | 0.84 | 0.86 | 0.83 | 0.84 | 0.83 | 0.84 | 0.87 | 0.83 | 0.85 |
| GAPDH | PFKFB3 | 0.83 | 0.83 | 0.84 | 0.82 | 0.84 | 0.82 | 0.84 | 0.85 | 0.80 | 0.83 |
| GAPDH | FFAR2 | 0.83 | 0.82 | 0.85 | 0.82 | 0.82 | 0.82 | 0.82 | 0.86 | 0.79 | 0.83 |
| GAPDH | FPR2 | 0.83 | 0.83 | 0.85 | 0.82 | 0.82 | 0.81 | 0.83 | 0.86 | 0.80 | 0.84 |
| GAPDH | HSPA1B | 0.81 | 0.81 | 0.83 | 0.80 | 0.81 | 0.80 | 0.81 | 0.84 | 0.74 | 0.82 |
| GAPDH | NT5C3 | 0.79 | 0.79 | 0.84 | 0.79 | 0.78 | 0.80 | 0.78 | 0.85 | 0.68 | 0.81 |
| GAPDH | DDX60L | 0.81 | 0.81 | 0.84 | 0.80 | 0.80 | 0.81 | 0.80 | 0.86 | 0.75 | 0.82 |
| GAPDH | SELL | 0.84 | 0.84 | 0.86 | 0.83 | 0.83 | 0.84 | 0.83 | 0.87 | 0.80 | 0.85 |
| GAPDH | IFITM1 | 0.82 | 0.83 | 0.85 | 0.83 | 0.81 | 0.83 | 0.82 | 0.86 | 0.77 | 0.84 |
| GAPDH | RAB24 | 0.80 | 0.80 | 0.82 | 0.79 | 0.79 | 0.79 | 0.79 | 0.84 | 0.73 | 0.81 |
| GAPDH | MCL1 | 0.81 | 0.80 | 0.83 | 0.79 | 0.79 | 0.80 | 0.80 | 0.85 | 0.73 | 0.81 |
| GAPDH | PROK2 | 0.84 | 0.83 | 0.85 | 0.82 | 0.85 | 0.82 | 0.84 | 0.86 | 0.82 | 0.84 |
| GAPDH | LILRA5 | 0.83 | 0.83 | 0.85 | 0.82 | 0.81 | 0.83 | 0.82 | 0.85 | 0.79 | 0.83 |
| GAPDH | TLR4 | 0.83 | 0.83 | 0.85 | 0.82 | 0.82 | 0.81 | 0.83 | 0.86 | 0.79 | 0.83 |
| | HKG | GAPDH | GAPDH | GAPDH | GAPDH | GAPDH | GAPDH | GAPDH | GAPDH | GAPDH | GAPDH |
| HKG | Gene | MAL1 | CCR7 | GZMK | FCER1A | FAIM3 | CD3D | CD6 | KLRB1 | IL7R | CCL5 |
| GAPDH | IL1RN | 0.86 | 0.87 | 0.86 | 0.86 | 0.86 | 0.87 | 0.86 | 0.86 | 0.87 | 0.87 |
| GAPDH | SLC22A4 | 0.85 | 0.86 | 0.85 | 0.85 | 0.86 | 0.86 | 0.84 | 0.86 | 0.85 | 0.85 |
| GAPDH | PLSCR1 | 0.86 | 0.88 | 0.87 | 0.86 | 0.87 | 0.88 | 0.86 | 0.87 | 0.87 | 0.87 |
| GAPDH | ANXA3 | 0.86 | 0.87 | 0.87 | 0.86 | 0.87 | 0.87 | 0.86 | 0.87 | 0.87 | 0.87 |
| GAPDH | LRG1 | 0.86 | 0.87 | 0.87 | 0.86 | 0.87 | 0.86 | 0.86 | 0.87 | 0.87 | 0.86 |
| GAPDH | C19ORF59 | 0.87 | 0.88 | 0.88 | 0.87 | 0.88 | 0.88 | 0.87 | 0.88 | 0.88 | 0.88 |
| GAPDH | ACSL1 | 0.86 | 0.87 | 0.87 | 0.87 | 0.87 | 0.87 | 0.87 | 0.87 | 0.87 | 0.87 |
| GAPDH | PFKFB3 | 0.85 | 0.86 | 0.86 | 0.86 | 0.86 | 0.86 | 0.85 | 0.86 | 0.86 | 0.86 |
| GAPDH | FFAR2 | 0.86 | 0.86 | 0.86 | 0.86 | 0.86 | 0.86 | 0.86 | 0.86 | 0.86 | 0.86 |
| GAPDH | FPR2 | 0.86 | 0.87 | 0.86 | 0.86 | 0.87 | 0.86 | 0.86 | 0.86 | 0.86 | 0.86 |
| GAPDH | HSPA1B | 0.84 | 0.85 | 0.84 | 0.84 | 0.85 | 0.85 | 0.84 | 0.85 | 0.85 | 0.85 |
| GAPDH | NT5C3 | 0.84 | 0.86 | 0.87 | 0.85 | 0.86 | 0.87 | 0.85 | 0.85 | 0.86 | 0.86 |
| GAPDH | DDX60L | 0.85 | 0.87 | 0.86 | 0.86 | 0.87 | 0.87 | 0.86 | 0.86 | 0.87 | 0.87 |
| GAPDH | SELL | 0.87 | 0.88 | 0.88 | 0.87 | 0.88 | 0.88 | 0.87 | 0.87 | 0.87 | 0.88 |
| GAPDH | IFITM1 | 0.86 | 0.87 | 0.87 | 0.86 | 0.87 | 0.88 | 0.86 | 0.86 | 0.87 | 0.87 |
| GAPDH | RAB24 | 0.84 | 0.85 | 0.85 | 0.84 | 0.85 | 0.85 | 0.83 | 0.85 | 0.85 | 0.85 |
| GAPDH | MCL1 | 0.85 | 0.86 | 0.86 | 0.86 | 0.86 | 0.86 | 0.85 | 0.86 | 0.86 | 0.86 |
| GAPDH | PROK2 | 0.87 | 0.87 | 0.87 | 0.86 | 0.87 | 0.87 | 0.87 | 0.87 | 0.86 | 0.87 |
| GAPDH | LILRA5 | 0.86 | 0.86 | 0.86 | 0.85 | 0.86 | 0.86 | 0.85 | 0.86 | 0.86 | 0.86 |
| GAPDH | TLR4 | 0.86 | 0.87 | 0.86 | 0.86 | 0.86 | 0.86 | 0.85 | 0.86 | 0.86. | 0.86 |
| | HKG | GAPDH | GAPDH | GAPDH | GAPDH | GAPDH | GAPDH | GAPDH | GAPDH | GAPDH | GAPDH |
| HKG | Gene | NFIL3 | IL1B | CYSTM1 | CSF2RB | IFITM3 | SOD2 | FCGR1B | S100A12 | SP100 | NAIP |
| GAPDH | NFIL3 | — | 0.83 | 0.84 | 0.83 | 0.82 | 0.82 | 0.83 | 0.86 | 0.79 | 0.84 |
| GAPDH | IL1B | — | — | 0.84 | 0.82 | 0.82 | 0.81 | 0.82 | 0.85 | 0.79 | 0.83 |
| GAPDH | CYSTM1 | — | — | — | 0.84 | 0.85 | 0.84 | 0.85 | 0.86 | 0.82 | 0.85 |
| GAPDH | CSF2RB | — | — | — | — | 0.82 | 0.81 | 0.82 | 0.85 | 0.79 | 0.83 |
| GAPDH | IFITM3 | — | — | — | — | — | 0.82 | 0.81 | 0.86 | 0.77 | 0.83 |
| GAPDH | SOD2 | — | — | — | — | — | — | 0.82 | 0.85 | 0.79 | 0.83 |
| GAPDH | FCGR1B | — | — | — | — | — | — | — | 0.85 | 0.78 | 0.83 |
| GAPDH | S100A12 | — | — | — | — | — | — | — | — | 0.84 | 0.86 |
| GAPDH | SP100 | — | — | — | — | — | — | — | — | — | 0.81 |
| GAPDH | NAIP | — | — | — | — | — | — | — | — | — | — |
| GAPDH | MAL1 | — | — | — | — | — | — | — | — | — | — |
| GAPDH | CCR7 | — | — | — | — | — | — | — | — | — | — |
| GAPDH | GZMK | — | — | — | — | — | — | — | — | — | — |
| GAPDH | FCER1A | — | — | — | — | — | — | — | — | — | — |
| GAPDH | FAIM3 | — | — | — | — | — | — | — | — | — | — |
| GAPDH | CD3D | — | — | — | — | — | — | — | — | — | — |
| GAPDH | CD6 | — | — | — | — | — | — | — | — | — | — |
| GAPDH | KLRB1 | — | — | — | — | — | — | — | — | — | — |
| GAPDH | IL7R | — | — | — | — | — | — | — | — | — | — |
| GAPDH | CCLS | — | — | — | — | — | — | — | — | — | — |
| | HKG | GAPDH | GAPDH | GAPDH | GAPDH | GAPDH | GAPDH | GAPDH | GAPDH | GAPDH | GAPDH |
| HKG | Gene | MAL1 | CCR7 | GZMK | FCER1A | FAIM3 | CD3D | CD6 | KLRB1 | IL7R | CCL5 |
| GAPDH | NFIL3 | 0.86 | 0.87 | 0.87 | 0.87 | 0.87 | 0.87 | 0.86 | 0.87 | 0.86 | 0.87 |
| GAPDH | IL1B | 0.85 | 0.86 | 0.86 | 0.86 | 0.86 | 0.86 | 0.85 | 0.86 | 0.85 | 0.86 |
| GAPDH | CYSTM1 | 0.87 | 0.88 | 0.87 | 0.87 | 0.87 | 0.87 | 0.87 | 0.88 | 0.87 | 0.87 |
| GAPDH | CSF2RB | 0.85 | 0.86 | 0.85 | 0.86 | 0.86 | 0.86 | 0.85 | 0.86 | 0.85 | 0.85 |
| GAPDH | IFITM3 | 0.86 | 0.87 | 0.87 | 0.86 | 0.87 | 0.87 | 0.86 | 0.86 | 0.87 | 0.87 |
| GAPDH | SOD2 | 0.85 | 0.86 | 0.85 | 0.85 | 0.85 | 0.85 | 0.84 | 0.85 | 0.85 | 0.85 |
| GAPDH | FCGR1B | 0.85 | 0.86 | 0.86 | 0.85 | 0.86 | 0.86 | 0.85 | 0.86 | 0.86 | 0.86 |
| GAPDH | S100A12 | 0.87 | 0.87 | 0.88 | 0.87 | 0.87 | 0.88 | 0.87 | 0.88 | 0.87 | 0.87 |
| GAPDH | SP100 | 0.84 | 0.85 | 0.85 | 0.84 | 0.85 | 0.86 | 0.84 | 0.85 | 0.85 | 0.85 |
| GAPDH | NAIP | 0.86 | 0.87 | 0.87 | 0.87 | 0.87 | 0.87 | 0.86 | 0.87 | 0.87 | 0.86 |
| GAPDH | MAL1 | — | 0.85 | 0.86 | 0.85 | 0.85 | 0.86 | 0.85 | 0.85 | 0.85 | 0.87 |
| GAPDH | CCR7 | — | — | 0.86 | 0.86 | 0.85 | 0.86 | 0.86 | 0.85 | 0.86 | 0.87 |
| GAPDH | GZMK | — | — | — | 0.85 | 0.86 | 0.85 | 0.86 | 0.86 | 0.86 | 0.86 |
| GAPDH | FCER1A | — | — | — | — | 0.85 | 0.86 | 0.86 | 0.84 | 0.86 | 0.86 |
| GAPDH | FAIM3 | — | — | — | — | — | 0.85 | 0.86 | 0.85 | 0.86 | 0.86 |
| GAPDH | CD3D | — | — | — | — | — | — | 0.86 | 0.85 | 0.86 | 0.86 |
| GAPDH | CD6 | — | — | — | — | — | — | — | 0.86 | 0.86 | 0.86 |
| GAPDH | KLRB1 | — | — | — | — | — | — | — | — | 0.86 | 0.86 |
| GAPDH | IL7R | — | — | — | — | — | — | — | — | — | 0.86 |
| GAPDH | CCLS | — | — | — | — | — | — | — | — | — | — |
| | HKG | GAPDH | GAPDH. | GAPDH | GAPDH | GAPDH | GAPDH | GAPDH | GAPDH | GAPDH | GAPDH |
| HKG | Gene | NFIL3 | IL1B | CYSTM1 | CSF2RB | IFITM3 | SOD2 | FCGR1B | S100A12 | SP100 | NAIP |
| HPRT1 | IL1RN | 0.83 | 0.82 | 0.85 | 0.81 | 0.82 | 0.81 | 0.82 | 0.85 | 0.79 | 0.83 |
| HPRT1 | SLC22A4 | 0.82 | 0.81 | 0.84 | 0.80 | 0.82 | 0.81 | 0.81 | 0.85 | 0.78 | 0.82 |
| HPRT1 | PLSCR1 | 0.84 | 0.83 | 0.85 | 0.83 | 0.82 | 0.83 | 0.82 | 0.86 | 0.80 | 0.84 |
| HPRT1 | ANXA3 | 0.84 | 0.82 | 0.84 | 0.81 | 0.83 | 0.81 | 0.82 | 0.84 | 0.80 | 0.83 |
| HPRT1 | LRG1 | 0.84 | 0.81 | 0.84 | 0.81 | 0.82 | 0.81 | 0.82 | 0.84 | 0.79 | 0.83 |
| HPRT1 | C19ORF59 | 0.85 | 0.83 | 0.85 | 0.83 | 0.84 | 0.83 | 0.83 | 0.85 | 0.81 | 0.84 |
| HPRT1 | ACSL1 | 0.84 | 0.82 | 0.84 | 0.81 | 0.83 | 0.82 | 0.83 | 0.85 | 0.80 | 0.83 |
| HPRT1 | PFKFB3 | 0.84 | 0.81 | 0.84 | 0.81 | 0.83 | 0.81 | 0.82 | 0.84 | 0.79 | 0.82 |
| HPRT1 | FFAR2 | 0.83 | 0.81 | 0.84 | 0.80 | 0.81 | 0.81 | 0.81 | 0.85 | 0.78 | 0.82 |
| HPRT1 | FPR2 | 0.84 | 0.82 | 0.84 | 0.81 | 0.82 | 0.81 | 0.82 | 0.85 | 0.79 | 0.83 |
| HPRT1 | HSPA1B | 0.85 | 0.83 | 0.85 | 0.83 | 0.84 | 0.83 | 0.84 | 0.86 | 0.82 | 0.84 |
| HPRT1 | NT5C3 | 0.82 | 0.81 | 0.84 | 0.81 | 0.80 | 0.81 | 0.80 | 0.85 | 0.76 | 0.82 |
| HPRT1 | DDX60L | 0.83 | 0.82 | 0.85 | 0.81 | 0.81 | 0.81 | 0.81 | 0.85 | 0.79 | 0.83 |
| HPRT1 | SELL | 0.84 | 0.83 | 0.85 | 0.82 | 0.83 | 0.82 | 0.83 | 0.85 | 0.81 | 0.84 |
| HPRT1 | IFITM1 | 0.83 | 0.82 | 0.85 | 0.81 | 0.81 | 0.82 | 0.81 | 0.85 | 0.79 | 0.83 |
| HPRT1 | RAB24 | 0.83 | 0.82 | 0.84 | 0.81 | 0.82 | 0.81 | 0.82 | 0.85 | 0.79 | 0.83 |
| HPRT1 | MCL1 | 0.83 | 0.81 | 0.84 | 0.81 | 0.82 | 0.81 | 0.82 | 0.85 | 0.79 | 0.83 |
| HPRT1 | PROK2 | 0.83 | 0.82 | 0.84 | 0.81 | 0.82 | 0.81 | 0.82 | 0.84 | 0.79 | 0.82 |
| HPRT1 | LILRA5 | 0.84 | 0.82 | 0.85 | 0.82 | 0.82 | 0.81 | 0.82 | 0.85 | 0.79 | 0.83 |
| HPRT1 | TLR4 | 0.83 | 0.81 | 0.84 | 0.81 | 0.82 | 0.81 | 0.82 | 0.85 | 0.79 | 0.82 |
| | HKG | GAPDH | GAPDH | GAPDH | GAPDH | GAPDH | GAPDH | GAPDH | GAPDH | GAPDH | GAPDH |
| HKG | Gene | MAL1 | CCR7 | GZMK | FCER1A | FAIM3 | CD3D | CD6 | KLRB1 | IL7R | CCL5 |
| HPRT1 | IL1RN | 0.84 | 0.85 | 0.84 | 0.84 | 0.84 | 0.84 | 0.84 | 0.84 | 0.85 | 0.85 |
| HPRT1 | SLC22A4 | 0.83 | 0.84 | 0.83 | 0.83 | 0.84 | 0.83 | 0.83 | 0.84 | 0.84 | 0.84 |
| HPRT1 | PLSCR1 | 0.84 | 0.85 | 0.85 | 0.84 | 0.85 | 0.85 | 0.84 | 0.85 | 0.85 | 0.85 |
| HPRT1 | ANXA3 | 0.84 | 0.84 | 0.84 | 0.83 | 0.84 | 0.84 | 0.84 | 0.84 | 0.84 | 0.84 |
| HPRT1 | LRG1 | 0.83 | 0.84 | 0.83 | 0.83 | 0.84 | 0.83 | 0.83 | 0.84 | 0.84 | 0.84 |
| HPRT1 | C19ORF59 | 0.84 | 0.85 | 0.84 | 0.84 | 0.85 | 0.85 | 0.84 | 0.85 | 0.85 | 0.85 |
| HPRT1 | ACSL1 | 0.84 | 0.85 | 0.84 | 0.84 | 0.84 | 0.84 | 0.84 | 0.84 | 0.85 | 0.85 |
| HPRT1 | PFKFB3 | 0.83 | 0.84 | 0.82 | 0.84 | 0.83 | 0.83 | 0.83 | 0.83 | 0.84 | 0.84 |
| HPRT1 | FFAR2 | 0.83 | 0.84 | 0.83 | 0.83 | 0.84 | 0.84 | 0.83 | 0.84 | 0.84 | 0.84 |
| HPRT1 | FPR2 | 0.84 | 0.85 | 0.83 | 0.83 | 0.84 | 0.84 | 0.84 | 0.84 | 0.85 | 0.84 |
| HPRT1 | HSPA1B | 0.85 | 0.86 | 0.85 | 0.85 | 0.86 | 0.85 | 0.85 | 0.86 | 0.86 | 0.86 |
| HPRT1 | NT5C3 | 0.83 | 0.85 | 0.85 | 0.84 | 0.85 | 0.85 | 0.84 | 0.84 | 0.85 | 0.85 |
| HPRT1 | DDX60L | 0.84 | 0.85 | 0.84 | 0.84 | 0.85 | 0.85 | 0.84 | 0.85 | 0.85 | 0.85 |
| HPRT1 | SELL | 0.84 | 0.85 | 0.85 | 0.84 | 0.85 | 0.85 | 0.85 | 0.85 | 0.85 | 0.85 |
| HPRT1 | IFITM1 | 0.84 | 0.85 | 0.84 | 0.84 | 0.85 | 0.85 | 0.84 | 0.84 | 0.85 | 0.85 |
| HPRT1 | RAB24 | 0.84 | 0.85 | 0.84 | 0.84 | 0.84 | 0.84 | 0.84 | 0.84 | 0.85 | 0.84 |
| HPRT1 | MCL1 | 0.84 | 0.84 | 0.83 | 0.84 | 0.84 | 0.84 | 0.84 | 0.84 | 0.84 | 0.84 |
| HPRT1 | PROK2 | 0.84 | 0.84 | 0.83 | 0.83 | 0.84 | 0.84 | 0.83 | 0.84 | 0.84 | 0.84 |
| HPRT1 | LILRA5 | 0.84 | 0.85 | 0.84 | 0.84 | 0.84 | 0.84 | 0.84 | 0.84 | 0.85 | 0.84 |
| HPRT1 | TLR4 | 0.83 | 0.84 | 0.83 | 0.84 | 0.84 | 0.83 | 0.83 | 0.83 | 0.84 | 0.84 |
| | HKG | GAPDH | GAPDH | GAPDH | GAPDH | GAPDH | GAPDH | GAPDH | GAPDH | GAPDH | GAPDH |
| HKG | Gene | NFIL3 | IL1B | CYSTM1 | CSF2RB | IFITM3 | SOD2 | FCGR1B | S100A12 | SP100 | NAIP |
| HPRT1 | NFIL3 | 0.83 | 0.82 | 0.85 | 0.82 | 0.83 | 0.82 | 0.82 | 0.85 | 0.80 | 0.83 |
| HPRT1 | IL1B | 0.83 | 0.81 | 0.84 | 0.81 | 0.82 | 0.81 | 0.82 | 0.85 | 0.79 | 0.83 |
| HPRT1 | CYSTM1 | 0.84 | 0.83 | 0.84 | 0.82 | 0.83 | 0.82 | 0.83 | 0.85 | 0.81 | 0.84 |
| HPRT1 | CSF2RB | 0.83 | 0.81 | 0.84 | 0.80 | 0.82 | 0.80 | 0.81 | 0.84 | 0.79 | 0.83 |
| HPRT1 | IFITM3 | 0.84 | 0.82 | 0.85 | 0.82 | 0.82 | 0.82 | 0.81 | 0.85 | 0.80 | 0.83 |
| HPRT1 | SOD2 | 0.83 | 0.81 | 0.84 | 0.81 | 0.82 | 0.80 | 0.82 | 0.84 | 0.79 | 0.83 |
| HPRT1 | FCGR1B | 0.83 | 0.82 | 0.84 | 0.81 | 0.81 | 0.81 | 0.81 | 0.85 | 0.79 | 0.83 |
| HPRT1 | S100A12 | 0.85 | 0.83 | 0.85 | 0.83 | 0.84 | 0.82 | 0.83 | 0.84 | 0.81 | 0.84 |
| HPRT1 | SP100 | 0.82 | 0.81 | 0.84 | 0.81 | 0.81 | 0.81 | 0.81 | 0.85 | 0.77 | 0.82 |
| HPRT1 | NAIP | 0.84 | 0.82 | 0.85 | 0.82 | 0.83 | 0.82 | 0.83 | 0.86 | 0.81 | 0.83 |
| HPRT1 | MAL1 | 0.83 | 0.82 | 0.84 | 0.82 | 0.81 | 0.82 | 0.82 | 0.84 | 0.78 | 0.82 |
| HPRT1 | CCR7 | 0.82 | 0.81 | 0.83 | 0.81 | 0.80 | 0.81 | 0.80 | 0.83 | 0.75 | 0.81 |
| HPRT1 | GZMK | 0.84 | 0.83 | 0.85 | 0.83 | 0.83 | 0.83 | 0.83 | 0.85 | 0.80 | 0.84 |
| HPRT1 | FCER1A | 0.83 | 0.82 | 0.85 | 0.83 | 0.81 | 0.82 | 0.81 | 0.84 | 0.77 | 0.83 |
| HPRT1 | FAIM3 | 0.81 | 0.83 | 0.84 | 0.83 | 0.82 | 0.83 | 0.82 | 0.84 | 0.79 | 0.83 |
| HPRT1 | CD3D | 0.85 | 0.84 | 0.85 | 0.84 | 0.84 | 0.84 | 0.84 | 0.86 | 0.81 | 0.84 |
| 11P811 | CD6 | 0.81 | 0.81 | 0.83 | 0.81 | 0.80 | 0.81 | 0.81 | 0.83 | 0.75 | 0.81 |
| HPRT1 | KLRB1 | 0.85 | 0.84 | 0.86 | 0.84 | 0.83 | 0.84 | 0.83 | 0.86 | 0.80 | 0.85 |
| HPRT1 | IL7R | 0.81 | 0.81 | 0.83 | 0.81 | 0.80 | 0.81 | 0.80 | 0.83 | 0.76 | 0.82 |
| HPRT1 | CCL5 | 0.83 | 0.83 | 0.85 | 0.83 | 0.82 | 0.83 | 0.83 | 0.85 | 0.79 | 0.84 |
| | HKG | GAPDH | GAPDH | GAPDH | GAPDH | GAPDH | GAPDH | GAPDH | GAPDH | GAPDH | GAPDH |
| HKG | Gene | MAL1 | CCR7 | GZMK | FCER1A | FAIM3 | CD3D | CD6 | KLRB1 | IL7R | CCL5 |
| HPRT1 | NFIL3 | 0.84 | 0.85 | 0.84 | 0.84 | 0.85 | 0.84 | 0.84 | 0.85 | 0.84 | 0.84 |
| HPRT1 | IL1B | 0.83 | 0.84 | 0.84 | 0.84 | 0.84 | 0.84 | 0.83 | 0.84 | 0.84 | 0.84 |
| HPRT1 | CYSTM1 | 0.84 | 0.85 | 0.84 | 0.84 | 0.85 | 0.85 | 0.85 | 0.85 | 0.85 | 0.85 |
| HPRT1 | CSF2RB | 0.83 | 0.84 | 0.83 | 0.84 | 0.84 | 0.84 | 0.84 | 0.84 | 0.84 | 0.84 |
| HPRT1 | IFITM3 | 0.84 | 0.85 | 0.84 | 0.84 | 0.85 | 0.85 | 0.84 | 0.85 | 0.85 | 0.85 |
| HPRT1 | SOD2 | 0.83 | 0.84 | 0.83 | 0.83 | 0.84 | 0.84 | 0.83 | 0.84 | 0.84 | 0.84 |
| HPRT1 | FCGR1B | 0.84 | 0.85 | 0.84 | 0.83 | 0.84 | 0.84 | 0.84 | 0.84 | 0.84 | 0.84 |
| HPRT1 | S100A12 | 0.84 | 0.85 | 0.84 | 0.84 | 0.85 | 0.85 | 0.84 | 0.84 | 0.85 | 0.85 |
| HPRT1 | SP100 | 0.84 | 0.85 | 0.84 | 0.84 | 0.85 | 0.84 | 0.84 | 0.84 | 0.85 | 0.84 |
| HPRT1 | NAIP | 0.84 | 0.85 | 0.84 | 0.84 | 0.85 | 0.84 | 0.84 | 0.85 | 0.85 | 0.85 |
| HPRT1 | MAL1 | 0.82 | 0.83 | 0.85 | 0.83 | 0.83. | 0.84 | 0.83 | 0.83 | 0.83 | 0.84 |
| HPRT1 | CCR7 | 0.81 | 0.81 | 0.83 | 0.82 | 0.82 | 0.83 | 0.81 | 0.82 | 0.82 | 0.83 |
| HPRT1 | GZMK | 0.84 | 0.85 | 0.83 | 0.84 | 0.84 | 0.84 | 0.84 | 0.84 | 0.84 | 0.85 |
| HPRT1 | FCER1A | 0.82 | 0.83 | 0.83 | 0.82 | 0.83 | 0.84 | 0.83 | 0.83 | 0.84 | 0.83 |
| HPRT1 | FAIM3 | 0.82 | 0.83 | 0.84 | 0.83 | 0.83 | 0.84 | 0.82 | 0.83 | 0.83 | 0.84 |
| HPRT1 | CD3D | 0.84 | 0.84 | 0.84 | 0.84 | 0.84 | 0.84 | 0.84 | 0.84 | 0.84 | 0.85 |
| 11P811 | CD6 | 0.82 | 0.83 | 0.84 | 0.83 | 0.83 | 0.84 | 0.82 | 0.83 | 0.83 | 0.83 |
| HPRT1 | KLRB1 | 0.83 | 0.84 | 0.84 | 0.83 | 0.84 | 0.84 | 0.83 | 0.83 | 0.84 | 0.84 |
| HPRT1 | IL7R | 0.82 | 0.82 | 0.83 | 0.83 | 0.82 | 0.84 | 0.82 | 0.83 | 0.82 | 0.83 |
| HPRT1 | CCL5 | 0.84 | 0.85 | 0.85 | 0.84 | 0.84 | 0.85 | 0.83 | 0.84 | 0.84 | 0.84 |

Table 26 below shows the weights given to each of the biomarkers of the biomarker panel of the 40 biomarkers or genes listed in List 1 for control/infection without SIRS/SIRS without infection versus mild sepsis/severe sepsis/septic shock.

TABLE 26
Weights were given to each of the biomarkers or genes of
the biomarker panel to allow the scoring algorithm for
segregating control/infection without SIRS/SIRS without
infection versus mild sepsis/severe sepsis/septic shock
(FIG. 4), with HPRT1/GAPDH as the housekeeping gene (n =
151, where “n” is the number of samples).

| No. | HKG | Gene | Weight |
|---|---|---|---|
| 1 | HPRT1 | IL1RN | −0.09 |
| 2 | HPRT1 | SLC22A4 | −0.12 |
| 3 | HPRT1 | PLSCR1 | −0.13 |
| 4 | HPRT1 | ANXA3 | −0.08 |
| 5 | HPRT1 | LRG1 | −0.07 |
| 6 | HPRT1 | C19ORF59 | −0.09 |
| 7 | HPRT1 | ACSL1 | −0.09 |
| 8 | HPRT1 | PFKFB3 | −0.10 |
| 9 | HPRT1 | FFAR2 | −0.08 |
| 10 | HPRT1 | FPR2 | −0.11 |
| 11 | HPRT1 | HSPA1B | −0.15 |
| 12 | HPRT1 | NT5C3 | −0.14 |
| 13 | HPRT1 | DDX60L | −0.13 |
| 14 | HPRT1 | SELL | −0.16 |
| 15 | HPRT1 | IFITM1 | −0.13 |
| 16 | HPRT1 | RAB24 | −0.16 |
| 17 | HPRT1 | MCL1 | −0.17 |
| 18 | HPRT1 | PROK2 | −0.08 |
| 19 | HPRT1 | LILRA5 | −0.12 |
| 20 | HPRT1 | TLR4 | −0.12 |
| 21 | HPRT1 | NFIL3 | −0.13 |
| 22 | HPRT1 | IL1B | −0.09 |
| 23 | HPRT1 | CYSTM1 | −0.10 |
| 24 | HPRT1 | CSF2RB | −0.11 |
| 25 | HPRT1 | IFITM3 | −0.13 |
| 26 | HPRT1 | SOD2 | −0.10 |
| 27 | HPRT1 | FCGR1B | −0.10 |
| 28 | HPRT1 | S100A12 | −0.10 |
| 29 | HPRT1 | SP100 | −0.16 |
| 30 | HPRT1 | NAIP | −0.12 |
| 31 | HPRT1 | MAL1 | 0.13 |
| 32 | HPRT1 | CCR7 | 0.15 |
| 33 | HPRT1 | GZMK | 0.15 |
| 34 | HPRT1 | FCER1A | 0.11 |
| 35 | HPRT1 | FAIM3 | 0.18 |
| 36 | HPRT1 | CD3D | 0.18 |
| 37 | HPRT1 | CD6 | 0.16 |
| 38 | HPRT1 | KLRB1 | 0.16 |
| 39 | HPRT1 | IL7R | 0.15 |
| 40 | HPRT1 | CCL5 | 0.17 |
| 41 | GAPDH | IL1RN | −0.13 |
| 42 | GAPDH | SLC22A4 | −0.16 |
| 43 | GAPDH | PLSCR1 | −0.16 |
| 44 | GAPDH | ANXA3 | −0.12 |
| 45 | GAPDH | LRG1 | −0.11 |
| 46 | GAPDH | C19ORF59 | −0.14 |
| 47 | GAPDH | ACSL1 | −0.13 |
| 48 | GAPDH | PFKFB3 | −0.16 |
| 49 | GAPDH | FFAR2 | −0.12 |
| 50 | GAPDH | FPR2 | −0.17 |
| 51 | GAPDH | HSPA1B | −0.13 |
| 52 | GAPDH | NT5C3 | −0.09 |
| 53 | GAPDH | DDX60L | −0.17 |
| 54 | GAPDH | SELL | −0.26 |
| 55 | GAPDH | IFITM1 | −0.19 |
| 56 | GAPDH | RAB24 | −0.20 |
| 57 | GAPDH | MCL1 | −0.26 |
| 58 | GAPDH | PROK2 | −0.12 |
| 59 | GAPDH | LILRA5 | −0.18 |
| 60 | GAPDH | TLR4 | −0.20 |
| 61 | GAPDH | NFIL3 | −0.20 |
| 62 | GAPDH | IL1B | −0.14 |
| 63 | GAPDH | CYSTM1 | −0.15 |
| 64 | GAPDH | CSF2RB | −0.16 |
| 65 | GAPDH | IFITM3 | −0.19 |
| 66 | GAPDH | SOD2 | −0.14 |
| 67 | GAPDH | FCGR1B | −0.13 |
| 68 | GAPDH | S100A12 | −0.16 |
| 69 | GAPDH | SP100 | −0.12 |
| 70 | GAPDH | NAIP | −0.20 |
| 71 | GAPDH | MAL1 | 0.12 |
| 72 | GAPDH | CCR7 | 0.17 |
| 73 | GAPDH | GZMK | 0.12 |
| 74 | GAPDH | FCER1A | 0.11 |
| 75 | GAPDH | FAIM3 | 0.15 |
| 76 | GAPDH | CD3D | 0.14 |
| 77 | GAPDH | CD6 | 0.15 |
| 78 | GAPDH | KLRB1 | 0.12 |
| 79 | GAPDH | IL7R | 0.15 |
| 80 | GAPDH | CCL5 | 0.14 |

Table 27 below shows the weights given to each of the biomarkers of the biomarker panel of the 40 biomarkers or genes listed in List 1 for mild sepsis versus severe sepsis/septic shock.

TABLE 27
Weights were given to each of the biomarkers or genes of
the biomarker panel for mild sepsis versus severe sepsis/septic
shock, (FIG. 5), with HPRT1/GAPDH as the housekeeping gene
(n = 85, where “n” is the number of samples).

| No. | HKG | Gene | Weight |
|---|---|---|---|
| 1 | HPRT1 | IL1RN | −0.06 |
| 2 | HPRT1 | SLC22A4 | 0.00 |
| 3 | HPRT1 | PLSCR1 | −0.09 |
| 4 | HPRT1 | ANXA3 | −0.06 |
| 5 | HPRT1 | LRG1 | −0.05 |
| 6 | HPRT1 | C19ORF59 | −0.07 |
| 7 | HPRT1 | ACSL1 | −0.06 |
| 8 | HPRT1 | PFKFB3 | −0.06 |
| 9 | HPRT1 | FFAR2 | −0.05 |
| 10 | HPRT1 | FPR2 | −0.07 |
| 11 | HPRT1 | HSPA1B | −0.06 |
| 12 | HPRT1 | NT5C3 | 0.00 |
| 13 | HPRT1 | DDX60L | −0.03 |
| 14 | HPRT1 | SELL | −0.06 |
| 15 | HPRT1 | IFITM1 | −0.08 |
| 16 | HPRT1 | RAB24 | −0.09 |
| 17 | HPRT1 | MCL1 | 0.00 |
| 18 | HPRT1 | PROK2 | −0.03 |
| 19 | HPRT1 | LILRA5 | −0.05 |
| 20 | HPRT1 | TLR4 | −0.07 |
| 21 | HPRT1 | NFIL3 | −0.08 |
| 22 | HPRT1 | IL1B | −0.05 |
| 23 | HPRT1 | CYSTM1 | −0.06 |
| 24 | HPRT1 | CSF2RB | −0.05 |
| 25 | HPRT1 | IFITM3 | −0.07 |
| 26 | HPRT1 | SOD2 | −0.07 |
| 27 | HPRT1 | FCGR1B | −0.08 |
| 28 | HPRT1 | S100A12 | −0.07 |
| 29 | HPRT1 | SP100 | −0.07 |
| 30 | HPRT1 | NAIP | −0.05 |
| 31 | HPRT1 | MAL1 | 0.06 |
| 32 | HPRT1 | CCR7 | 0.10 |
| 33 | HPRT1 | GZMK | 0.10 |
| 34 | HPRT1 | FCER1A | 0.09 |
| 35 | HPRT1 | FAIM3 | 0.12 |
| 36 | HPRT1 | CD3D | 0.12 |
| 37 | HPRT1 | CD6 | 0.09 |
| 38 | HPRT1 | KLRB1 | 0.09 |
| 39 | HPRT1 | IL7R | 0.08 |
| 40 | HPRT1 | CCL5 | 0.07 |
| 41 | GAPDH | IL1RN | −0.05 |
| 42 | GAPDH | SLC22A4 | 0.00 |
| 43 | GAPDH | PLSCR1 | 0.00 |
| 44 | GAPDH | ANXA3 | −0.06 |
| 45 | GAPDH | LRG1 | −0.06 |
| 46 | GAPDH | C19ORF59 | −0.08 |
| 47 | GAPDH | ACSL1 | −0.08 |
| 48 | GAPDH | PFKFB3 | −0.05 |
| 49 | GAPDH | FFAR2 | 0.00 |
| 50 | GAPDH | FPR2 | −0.09 |
| 51 | GAPDH | HSPA1B | −0.05 |
| 52 | GAPDH | NT5C3 | 0.00 |
| 53 | GAPDH | DDX60L | 0.00 |
| 54 | GAPDH | SELL | 0.00 |
| 55 | GAPDH | IFITM1 | −0.04 |
| 56 | GAPDH | RAB24 | −0.07 |
| 57 | GAPDH | MCL1 | 0.00 |
| 58 | GAPDH | PROK2 | −0.03 |
| 59 | GAPDH | LILRA5 | 0.00 |
| 60 | GAPDH | TLR4 | −0.08 |
| 61 | GAPDH | NFIL3 | −0.07 |
| 62 | GAPDH | IL1B | 0.00 |
| 63 | GAPDH | CYSTM1 | −0.07 |
| 64 | GAPDH | CSF2RB | −0.06 |
| 65 | GAPDH | IFITM3 | 0.00 |
| 66 | GAPDH | SOD2 | −0.08 |
| 67 | GAPDH | FCGR1B | −0.08 |
| 68 | GAPDH | S100A12 | −0.08 |
| 69 | GAPDH | SP100 | 0.00 |
| 70 | GAPDH | NAIP | 0.00 |
| 71 | GAPDH | MAL1 | 0.07 |
| 72 | GAPDH | CCR7 | 0.10 |
| 73 | GAPDH | GZMK | 0.08 |
| 74 | GAPDH | FCER1A | 0.08 |
| 75 | GAPDH | FAIM3 | 0.10 |
| 76 | GAPDH | CD3D | 0.08 |
| 77 | GAPDH | CD6 | 0.09 |
| 78 | GAPDH | KLRB1 | 0.08 |
| 79 | GAPDH | IL7R | 0.09 |
| 80 | GAPDH | CCL5 | 0.07 |

In some embodiments, the methods or kits respectively described herein use any five of the 40 biomarkers or genes listed in List 1.

Table 28 below shows the predictive value (Area Under Curve (AUC)) of exemplary sets of five biomarkers of the biomarker panel of the 40 biomarkers or genes listed in List 1 for control versus sepsis, with HPRT1/GAPDH as the housekeeping gene.

TABLE 28
Predictive value (AUC) of exemplary sets of five biomarkers or genes of
the biomarker panel for control versus sepsis, with HPRTI/GAPDH as the
housekeeping gene.

| Gene1 | Gene2 | Gene3 | Gene4 | Gene5 | Specificity | Sensitivity | AUC |
|---|---|---|---|---|---|---|---|
| IFITM3 | SELL | MCL1 | FPR2 | CD3D | 0.73 | 0.87 | 0.84 |
| FPR2 | NT5C3 | CCL5 | HSPA1B | SLC22A4 | 0.72 | 0.94 | 0.84 |
| S100A12 | HSPA1B | CCL5 | ACSL1 | CD6 | 0.74 | 0.90 | 0.84 |
| FAIM3 | CYSTM1 | KLRB1 | SLC22A4 | MAL1 | 0.74 | 0.89 | 0.84 |
| CSF2RB | KLRB1 | IL1RN | SP100 | CYSTM1 | 0.70 | 0.91 | 0.84 |
| FFAR2 | HSPA1B | CCL5 | IL7R | CYSTM1 | 0.75 | 0.87 | 0.84 |
| IL7R | CYSTM1 | S100A12 | C19ORF59 | ANXA3 | 0.74 | 0.86 | 0.84 |
| RAB24 | DDX60L | CYSTM1 | KLRB1 | PFKFB3 | 0.72 | 0.90 | 0.84 |
| SELL | CYSTM1 | HSPA1B | MCL1 | CCL5 | 0.78 | 0.84 | 0.85 |
| ACSL1 | CD6 | GZMK | HSPA1B | PFKFB3 | 0.72 | 0.91 | 0.84 |
| MAL1 | RAB24 | HSPA1B | IL7R | CCL5 | 0.73 | 0.90 | 0.85 |
| NAIP | HSPA1B | CYSTM1 | IL7R | CCL5 | 0.74 | 0.89 | 0.84 |
| PROK2 | KLRB1 | HSPA1B | NAIP | FPR2 | 0.74 | 0.86 | 0.84 |
| IFITM1 | KLRB1 | GZMK | TLR4 | HSPA1B | 0.72 | 0.89 | 0.85 |
| NT5C3 | HSPA1B | PROK2 | C19ORF59 | FFAR2 | 0.72 | 0.93 | 0.84 |
| PFKFB3 | SLC22A4 | LILRA5 | HSPA1B | KLRB1 | 0.78 | 0.80 | 0.85 |
| TLR4 | ACSL1 | DDX60L | FAIM3 | HSPA1B | 0.72 | 0.90 | 0.84 |
| FCER1A | CCL5 | HSPA1B | CYSTM1 | C19ORF59 | 0.73 | 0.91 | 0.85 |
| KLRB1 | CCL5 | HSPA1B | NT5C3 | FCGR1B | 0.74 | 0.90 | 0.84 |
| C19ORF59 | FPR2 | CD6 | HSPA1B | PFKFB3 | 0.73 | 0.90 | 0.85 |
| CYSTM1 | MAL1 | HSPA1B | CCL5 | IL7R | 0.73 | 0.89 | 0.85 |
| DDX60L | CSF2RB | HSPA1B | CCL5 | FFAR2 | 0.73 | 0.94 | 0.84 |
| GZMK | TLR4 | HSPA1B | C19ORF59 | IL1RN | 0.72 | 0.94 | 0.84 |
| ANXA3 | IL7R | CCR7 | KLRB1 | HSPA1B | 0.75 | 0.83 | 0.84 |
| CCR7 | FPR2 | KLRB1 | CYSTM1 | MCL1 | 0.73 | 0.89 | 0.84 |
| IL1RN | IL7R | CCR7 | KLRB1 | CYSTM1 | 0.72 | 0.90 | 0.84 |
| LILRA5 | TLR4 | KLRB1 | HSPA1B | CD6 | 0.78 | 0.83 | 0.85 |
| CD3D | HSPA1B | IL1RN | RAB24 | SELL | 0.75 | 0.89 | 0.84 |
| CD6 | PFKFB3 | LILRA5 | CCL5 | HSPA1B | 0.73 | 0.93 | 0.85 |
| HSPA1B | PFKFB3 | CD6 | DDX60L | CCL5 | 0.72 | 0.93 | 0.85 |
| IL1B | CCL5 | HSPA1B | FCGR1B | TLR4 | 0.72 | 0.93 | 0.85 |
| MCL1 | CYSTM1 | KLRB1 | C19ORF59 | HSPA1B | 0.74 | 0.86 | 0.84 |
| LRG1 | IL1RN | C19ORF59 | HSPA1B | NFIL3 | 0.73 | 0.90 | 0.84 |
| PLSCR1 | SOD2 | HSPA1B | IL7R | CCL5 | 0.72 | 0.94 | 0.85 |
| CCL5 | HSPA1B | CD6 | ANXA3 | FAIM3 | 0.70 | 0.90 | 0.85 |
| FCGR1B | KLRB1 | PLSCR1 | CYSTM1 | CCR7 | 0.73 | 0.87 | 0.84 |
| NFIL3 | S100A12 | HSPA1B | LILRA5 | IFITM3 | 0.77 | 0.84 | 0.84 |
| SOD2 | HSPA1B | CSF2RB | KLRB1 | FCGR1B | 0.75 | 0.83 | 0.84 |
| SLC22A4 | HSPA1B | GZMK | CYSTM1 | FCGR1B | 0.73 | 0.90 | 0.84 |
| SP100 | HSPA1B | CCR7 | GZMK | CD3D | 0.73 | 0.87 | 0.84 |

In some embodiments, the methods or kits respectively described herein use any ten of the 40 biomarkers or genes listed in List 1.

Table 29 below shows the predictive value (Area Under Curve (AUC)) of exemplary sets of ten biomarkers of the biomarker panel of the 40 biomarkers or genes listed in List 1 for control versus sepsis, with HPRT1/GAPDH as the housekeeping gene.

TABLE 29
Predictive value (AUC) of exemplary sets of ten biomarkers or genes of the biomarker panel for control versus sepsis,
with HPRT1/GAPDH as the housekeeping gene.

| Gene1 | Gene2 | Gene3 | Gene4 | GeneS | Gene6 | Gene7 | Gene8 | Gene9 | Gene10 | Specificity | Sensitivity | AUC |
|---|---|---|---|---|---|---|---|---|---|---|---|---|
| ACSL1 | HSPA1B | SOD2 | ANXA3 | IFITM3 | FPR2 | RAB24 | CYSTM1 | C19ORF59 | CD3D | 0.72 | 0.97 | 0.85 |
| ANXA3 | HSPA1B | SLC22A4 | NT5C3 | C19ORF59 | PROK2 | CYSTM1 | NFIL3 | TLR4 | FCGR1B | 0.73 | 0.93 | 0.86 |
| C19ORF59 | PFKFB3 | NT5C3 | IL7R | HSPA1B | FFAR2 | GZMK | IFITM3 | ACSL1 | FPR2 | 0.70 | 0.94 | 0.86 |
| CCL5 | SLC22A4 | CD6 | IL7R | NFIL3 | FCGR1B | HSPA1B | TLR4 | IL1B | IL1RN | 0.70 | 0.94 | 0.86 |
| CCR7 | FPR2 | MCL1 | TLR4 | IFITM1 | ANXA3 | PLSCR1 | MAL1 | HSPA1B | CSF2RB | 0.73 | 0.89 | 0.85 |
| CD3D | LILRA5 | C19ORF59 | FCER1A | SELL | MCL1 | HSPA1B | DDX60L | PFKFB3 | CYSTM1 | 0.69 | 0.94 | 0.86 |
| CD6 | C19ORF59 | TLR4 | FCGR1B | MAL1 | KLRB1 | HSPA1B | SLC22A4 | CCL5 | PLSCR1 | 0.63 | 0.99 | 0.86 |
| CSF2RB | C19ORF59 | KLRB1 | IFITM1 | SP100 | TLR4 | CCL5 | IFITM3 | HSPA1B | NFIL3 | 0.77 | 0.89 | 0.87 |
| CYSTM1 | FCGR1B | MAL1 | PROK2 | TLR4 | FPR2 | IL1RN | CD3D | HSPA1B | ACSL1 | 0.67 | 0.99 | 0.85 |
| DDX60L | S100A12 | CCR7 | TLR4 | SP100 | CSF2RB | HSPA1B | RAB24 | LRG1 | SOD2 | 0.73 | 0.90 | 0.85 |
| FAIM3 | IFITM1 | MCL1 | HSPA1B | LRG1 | CYSTM1 | TLR4 | CCR7 | CSF2RB | FPR2 | 0.70 | 0.93 | 0.85 |
| FCER1A | LILRA5 | CYSTM1 | NFIL3 | HSPA1B | C19ORF59 | NAIP | LRG1 | SELL | CSF2RB | 0.73 | 0.91 | 0.85 |
| FCGR1B | MCL1 | NAIP | LRG1 | GZMK | DDX60L | PFKFB3 | HSPA1B | PROK2 | IFITM3 | 0.70 | 0.90 | 0.85 |
| FFAR2 | FCER1A | IL1B | TLR4 | ANXA3 | CCL5 | ACSL1 | IL1RN | SLC22A4 | HSPA1B | 0.77 | 0.91 | 0.86 |
| FPR2 | NAIP | FFAR2 | SELL | IFITM1 | PLSCR1 | CD3D | PFKFB3 | TLR4 | CYSTM1 | 0.70 | 0.94 | 0.85 |
| GZMK | C19ORF59 | LRG1 | DDX60L | LILRA5 | FCGR1B | TLR4 | HSPA1B | S100A12 | SP100 | 0.72 | 0.93 | 0.86 |
| HSPA1B | KLRB1 | TLR4 | RAB24 | CCL5 | NAIP | MAL1 | IL7R | FCER1A | IFITM3 | 0.73 | 0.96 | 0.87 |
| IFITM1 | CD6 | SELL | CCR7 | FCGR1B | SP100 | PROK2 | HSPA1B | TLR4 | IFITM3 | 0.70 | 0.96 | 0.87 |
| IFITM3 | FCGR1B | PROK2 | HSPA1B | CCL5 | IL7R | C19ORF59 | TLR4 | FFAR2 | IL1B | 0.72 | 0.94 | 0.86 |
| IL1B | IL1RN | C19ORF59 | ANXA3 | LILRA5 | HSPA1B | CYSTM1 | KLRB1 | S100A12 | TLR4 | 0.77 | 0.89 | 0.86 |
| IL1RN | CSF2RB | SOD2 | HSPA1B | IFITM1 | SELL | MCL1 | FFAR2 | CCL5 | PROK2 | 0.74 | 0.90 | 0.85 |
| IL7R | CSF2RB | HSPA1B | TLR4 | CD3D | CCL5 | FFAR2 | RAB24 | CYSTM1 | MAL1 | 0.73 | 0.93 | 0.86 |
| KLRB1 | FPR2 | CCR7 | CYSTM1 | RAB24 | CCL5 | SP100 | LILRA5 | S100A12 | SELL | 0.74 | 0.89 | 0.85 |
| LILRA5 | LRG1 | MCL1 | DDX60L | CD3D | 1L1RN | SELL | HSPA1B | ANXA3 | IL1B | 0.74 | 0.90 | 0.85 |
| LRG1 | TLR4 | CD3D | SLC22A4 | MAL1 | ANXA3 | IFITM3 | HSPA1B | SP100 | S100A12 | 0.75 | 0.91 | 0.86 |
| MAL1 | CYSTM1 | SELL | IFITM1 | TLR4 | SOD2 | CCR7 | FPR2 | HSPA1B | CCL5 | 0.72 | 0.94 | 0.85 |
| NAIP | NFIL3 | CCR7 | IFITM1 | KLRB1 | TLR4 | LRG1 | PLSCR1 | FCER1A | FPR2 | 0.64 | 0.97 | 0.85 |
| NFIL3 | CSF2RB | SOD2 | TLR4 | SLC22A4 | ANXA3 | C19ORF59 | IL1B | IL7R | HSPA1B | 0.69 | 0.93 | 0.86 |
| MCL1 | ANXA3 | LRG1 | SP100 | S100A12 | CD3D | SELL | FCGR1B | PROK2 | PLSCR1 | 0.74 | 0.91 | 0.84 |
| NT5C3 | MCL1 | LRG1 | S100A12 | HSPA1B | DDX60L | IL1RN | IL1B | C19ORF59 | LILRA5 | 0.77 | 0.86 | 0.85 |
| PFKFB3 | TLR4 | ACSL1 | PROK2 | CCR7 | ANXA3 | RAB24 | CYSTM1 | HSPA1B | GZMK | 0.72 | 0.90 | 0.86 |
| PLSCR1 | S100A12 | TLR4 | SP100 | 1L7R | MAL1 | GZMK | IFITM1 | KLRB1 | ACSL1 | 0.73 | 0.90 | 0.84 |
| PROK2 | FCGR1B | NFIL3 | HSPA1B | CCL5 | IFITM3 | TLR4 | FPR2 | C19ORF59 | SELL | 0.74 | 0.91 | 0.86 |
| RAB24 | SELL | HSPA1B | CCR7 | IL1B | TLR4 | NAIP | IL1RN | ACSL1 | CYSTM1 | 0.74 | 0.96 | 0.86 |
| S100A12 | CCL5 | IL1B | LILRA5 | NAIP | CYSTM1 | SELL | IL1RN | TLR4 | IFITM1 | 0.74 | 0.90 | 0.85 |
| SELL | KLRB1 | MCL1 | CD6 | LRG1 | CCR7 | GZMK | HSPA1B | NT5C3 | IFITM3 | 0.74 | 0.87 | 0.85 |
| SLC22A4 | HSPA1B | IL7R | CYSTM1 | CCL5 | ACSL1 | FAIM3 | LRG1 | PLSCR1 | RAB24 | 0.74 | 0 91 | 0.86 |
| SOD2 | CCR7 | C19ORF59 | IFITM1 | RAB24 | NAIP | CYSTM1 | SELL | PFKFB3 | SLC22A4 | 0.74 | 0.91 | 0.85 |
| SP100 | FPR2 | NAIP | LILRA5 | CD6 | FFAR2 | IFITM3 | CSF2RB | TLR4 | HSPA1B | 0.75 | 0.90 | 0.87 |
| TLR4 | C19ORF59 | IL1B | FAIM3 | IFITM3 | HSPA1B | GZMK | ACSL1 | CCR7 | SP100 | 0.75 | 0.91 | 0.86 |

In some embodiments, the methods or kits respectively described herein use any twenty of the 40 biomarker's or genes listed in List 1.

Table 30 below shows the predictive value (Area Under Curve (AUC)) of exemplary sets of twenty biomarkers of the biomarker panel of the 40 biomarkers or genes listed in List 1 for control versus sepsis, with HPRT1/GAPDH as the housekeeping gene.

TABLE 30
Predictive value (AUC) of exemplary sets of twenty biomarkers or genes of the biomarker panel for control versus sepsis,
with HPRT1/GAPDH as the housekeeping gene.

| Gene1 | ACSL1 | ANXA3 | C19ORF59 | CCL5 | CCR7 | CD3D | CD6 | CSF2RB | CYSTM1 | DDX60L |
|---|---|---|---|---|---|---|---|---|---|---|
| Gene2 | IFITM1 | C19ORF59 | PFKFB3 | FCER1A | HSPA1B | SELL | PLSCR1 | PROK2 | SOD2 | PROK2 |
| Gene3 | CSF2RB | MCL1 | MAL1 | ANXA3 | FCGR1B | NFIL3 | IFITM1 | FFAR2 | KLRB1 | NAIP |
| Gene4 | HSPA1B | PFKFB3 | IFITM3 | SP100 | SLC22A4 | GZMK | IL7R | MCL1 | SP100 | HSPA1B |
| Gene5 | PFKFB3 | TLR4 | HSPA1B | HSPA1B | TLR4 | IL7R | KLRB1 | CD3D | HSPA1B | FCGR1B |
| Gene6 | FPR2 | CCL5 | KLRB1 | NAIP | PFKFB3 | HSPA1B | C19ORF59 | CCL5 | IL7R | IFITM3 |
| Gene7 | RAB24 | DDX60L | SP100 | PROK2 | NFIL3 | IFITM1 | ANXA3 | IFITM1 | ANXA3 | SLC22A4 |
| Gene8 | ANXA3 | NT5C3 | TLR4 | CD6 | CD3D | PROK2 | SOD2 | ANXA3 | FAIM3 | MAL1 |
| Gene9 | PLSCR1 | NAIP | IFITM1 | DDX60L | GZMK | NAIP | TLR4 | TLR4 | RAB24 | S100A12 |
| Gene10 | FCER1A | LILRA5 | CCR7 | PFKFB3 | IL1RN | ACSL1 | NT5C3 | CYSTM1 | TLR4 | NT5C3 |
| Gene11 | CD3D | IFITM3 | LRG1 | SOD2 | FCER1A | IFITM3 | IFITM3 | PLSCR1 | NT5C3 | TLR4 |
| Gene12 | FFAR2 | IL1RN | FCGR1B | TLR4 | SOD2 | CCL5 | SLC22A4 | IFITM3 | IFITM1 | CCL5 |
| Gene13 | NAIP | FAIM3 | CD6 | IL7R | SP100 | C19ORF59 | SP100 | FCER1A | PLSCR1 | IFITM1 |
| Gene14 | KLRB1 | CCR7 | RAB24 | KLRB1 | KLRB1 | ANXA3 | NAIP | SP100 | CCL5 | SELL |
| Gene15 | MAL1 | LRG1 | MCL1 | CD3D | ACSL1 | PFKFB3 | CYSTM1 | PFKFB3 | FPR2 | IL1B |
| Gene16 | TLR4 | NFIL3 | CYSTM1 | CCR7 | IFITM3 | TLR4 | FAIM3 | IL1B | FCGR1B | FPR2 |
| Gene17 | CYSTM1 | HSPA1B | NAIP | IFITM1 | LRG1 | FCGR1B | CCL5 | CD6 | NAIP | PFKFB3 |
| Gene18 | CD6 | SP100 | NT5C3 | CYSTM1 | IL1B | SP100 | DDX60L | C19ORF59 | LRG1 | C19ORF59 |
| Gene19 | CCL5 | SLC22A4 | CCL5 | S100A12 | MCL1 | PLSCR1 | IL1RN | HSPA1B | SELL | CCR7 |
| Gene20 | IFITM3 | IFITM1 | IL1RN | IFITM3 | C19ORF59 | RAB24 | HSPA1B | GZMK | IFITM3 | FAIM3 |
| Specificity | 0.74 | 0.75 | 0.75 | 0.78 | 0.80 | 0.75 | 0.80 | 0.75 | 0.77 | 0.75 |
| Sensitivity | 0.93 | 0.90 | 0.94 | 0.91 | 0.86 | 0.94 | 0.89 | 0.94 | 0.93 | 0.90 |
| AUC | 0.89 | 0.89 | 0.89 | 0.89 | 0.89 | 0.89 | 0.89 | 0.89 | 0.89 | 0.89 |
| Gene1 | FAIM3 | FCER1A | FCGR1B | FFAR2 | FPR2 | GZMK | HSPA1B | IFITM1 | IFITM3 | IL1B |
| Gene2 | C19ORF59 | PROK2 | SLC22A4 | LRG1 | KLRB1 | CCL5 | MAL1 | FFAR2 | ACSL1 | PFKFB3 |
| Gene3 | HSPA1B | SOD2 | IFITM3 | MAL1 | RAB24 | TLR4 | NFIL3 | TLR4 | RAB24 | ANXA3 |
| Gene4 | CSF2RB | PFKFB3 | TLR4 | IL1RN | C19ORF59 | CD3D | CCL5 | PLSCR1 | SOD2 | KLRB1 |
| Gene5 | IL7R | MCL1 | FAIM3 | IFITM3 | CYSTM1 | S100A12 | GZMK | C19ORF59 | TLR4 | IFITM1 |
| Gene6 | LILRA5 | SP100 | PLSCR1 | C19ORF59 | CD6 | CCR7 | LRG1 | CCL5 | NAIP | TLR4 |
| Gene7 | MCL1 | IFITM3 | NT5C3 | NFIL3 | NAIP | IL1RN | SLC22A4 | SLC22A4 | S100A12 | FFAR2 |
| Gene8 | IFITM1 | KLRB1 | KLRB1 | HSPA1B | FCER1A | C19ORF59 | KLRB1 | LILRA5 | PLSCR1 | HSPA1B |
| Gene9 | SP100 | DDX60L | C19ORF59 | IL1B | IFITM3 | RAB24 | S100A12 | FAIM3 | IL1RN | PLSCR1 |
| Gene10 | PROK2 | TLR4 | FFAR2 | PROK2 | FAIM3 | IL7R | PFKFB3 | LRG1 | FPR2 | SP100 |
| Gene11 | IFITM3 | IL7R | IL1B | DDX60L | MCL1 | MCL1 | CYSTM1 | DDX60L | SP100 | CCR7 |
| Gene12 | SLC22A4 | C19ORF59 | HSPA1B | TLR4 | PFKFB3 | NAIP | DDX60L | HSPA1B | CCR7 | RAB24 |
| Gene13 | PLSCR1 | LRG1 | SP100 | IFITM1 | CD3D | NT5C3 | IFITM1 | SP100 | NFIL3 | CD3D |
| Gene14 | CD3D | IFITM1 | SOD2 | SP100 | PROK2 | LILRA5 | FFAR2 | IFITM3 | CD3D | MAL1 |
| Gene15 | SELL | HSPA1B | CSF2RB | CCL5 | ANXA3 | IFITM1 | C19ORF59 | PFKFB3 | CCL5 | IFITM3 |
| Gene16 | CCL5 | RAB24 | MCL1 | NAIP | HSPA1B | HSPA1B | TLR4 | ANXA3 | SELL | SOD2 |
| Gene17 | NAIP | PLSCR1 | SELL | ACSL1 | TLR4 | LRG1 | PROK2 | MAL1 | PROK2 | FCER1A |
| Gene18 | TLR4 | CCR7 | ANXA3 | SOD2 | CCR7 | PFKFB3 | NAIP | IL7R | LRG1 | ACSL1 |
| Gene19 | KLRB1 | CD6 | RAB24 | MCL1 | IFITM1 | SP100 | IFITM3 | CD6 | HSPA1B | LRG1 |
| Gene20 | FCGR1B | LILRA5 | IFITM1 | LILRA5 | CCL5 | IFITM3 | SP100 | ACSL1 | IFITM1 | C19ORF59 |
| Specificity | 0.80 | 0.74 | 0.74 | 0.80 | 0.78 | 0.78 | 0.79 | 0.78 | 0.79 | 0.74 |
| Sensitivity | 0.87 | 0.97 | 0.96 | 0.87 | 0.90 | 0.93 | 0.90 | 0.90 | 0.94 | 0.94 |
| AUC | 0.89 | 0.89 | 0.89 | 0.89 | 0.89 | 0.89 | 0.89 | 0.89 | 0.89 | 0.89 |
| Gene1 | IL1RN | IL7R | KLRB1 | LILRA5 | LRG1 | MALI. | MCL1 | NAIP | NFIL3 | NT5C3 |
| Gene2 | HSPA1B | FCGR1B | MAL1 | SLC22A4 | IL1B | S100A12 | C19ORF59 | IL1B | SELL | HSPA1B |
| Gene3 | NT5C3 | CCL5 | CCL5 | SP100 | S100A12 | NFIL3 | RAB24 | NFIL3 | FCER1A | LRG1 |
| Gene4 | LRG1 | FAIM3 | HSPA1B | CCL5 | HSPA1B | FPR2 | ANXA3 | TLR4 | GZMK | NAIP |
| Gene5 | ACSL1 | LRG1 | NFIL3 | MAL1 | NAIP | SLC22A4 | CSF2RB | FCER1A | MAL1 | SLC22A4 |
| Gene6 | CYSTM1 | RAB24 | RAB24 | IFITM3 | PLSCR1 | TLR4 | CCL5 | FPR2 | CCL5 | LILRA5 |
| Gene7 | IFITM3 | PROK2 | IFITM1 | PROK2 | PROK2 | NAIP | IFITM1 | SLC22A4 | NAIP | TLR4 |
| Gene8 | FAIM3 | NAIP | IL1B | TLR4 | CCL5 | CCR7 | SLC22A4 | SELL | C19ORF59 | SOD2 |
| Gene9 | MCL1 | LILRA5 | SELL | SELL | IL7R | HSPA1B | IFITM3 | KLRB1 | ACSL1 | PFKFB3 |
| Gene10 | CD6 | HSPA1B | LILRA5 | DDX60L | C19ORF59 | KLRB1 | DDX60L | SP100 | TLR4 | ACSL1 |
| Gene11 | RAB24 | CD6 | IFITM3 | FPR2 | GZMK | IFITM3 | SOD2 | LRG1 | NT5C3 | IL1B |
| Gene12 | CCR7 | IL1RN | NAIP | ACSL1 | SELL | CYSTM1 | CYSTM1 | IL7R | HSPA1B | CSF2RB |
| Gene13 | SP100 | SP100 | S100A12 | HSPA1B | SP100 | IL1RN | PFKFB3 | PFKFB3 | SP100 | FFAR2 |
| Gene14 | IFITM1 | TLR4 | TLR4 | FAIM3 | FCGR1B | PFKFB3 | S100Al2 | IFITM3 | IFITM3 | C190RF59 |
| Gene15 | NAIP | CSF2RB | ACSL1 | LRG1 | TLR4 | IL1B | TLR4 | CD6 | PFKFB3 | SP100 |
| Gene16 | SELL | IFITM1 | NT5C3 | GZMK | RAB24. | PLSCR1 | CCR7 | LILRA5 | IFITM1 | IFITM3 |
| Gene17 | CCL5 | SELL | LRG1 | PFKFB3 | SOD2 | IFITM1 | NAIP | IL1RN | CD6 | FAIM3 |
| Gene18 | TLR4 | FFAR2 | PROK2 | IL1RN | IFITM3 | ACSL1 | KLRB1 | HSPA1B | RAB24 | GZMK |
| Gene19 | PLSCR1 | IFITM3 | SP100 | IL7R | FPR2 | ANXA3 | CD6 | CYSTM1 | LILRA5 | KLRB1 |
| Gene20 | NFIL3 | SOD2 | IL7R | KLRB1 | MAL1 | C19ORF59 | HSPA1B | ACSL1 | IL1RN | ANXA3 |
| Specificity | 0.78 | 0.74 | 0.79 | 0.75 | 0.77 | 0.79 | 0.77 | 0.77 | 0.75 | 0.79 |
| Sensitivity | 0.94 | 0.94 | 0.94 | 0.90 | 0.90 | 0.87 | 0.90 | 0.90 | 0.96 | 0.89 |
| AUC | 0.89 | 0.89 | 0.89 | 0.89 | 0.89 | 0.89 | 0.89 | 0.89 | 0.89 | 0.89 |
| Gene1 | PFKFB3 | PLSCR1 | PROK2 | RAB24 | S100A12 | SELL | SLC22A4 | SOD2 | SP100 | TLR4 |
| Gene2 | DDX60L | FAIM3 | MCL1 | SLC22A4 | GZMK | NFIL3 | KLRB1 | RAB24 | ACSL1 | CCL5 |
| Gene3 | CSF2RB | C19ORF59 | HSPA1B | NAIP | SOD2 | IFITM1 | LRG1 | IL1B | IFITM3 | IFITM1 |
| Gene4 | LRG1 | ACSL1 | C19ORF59 | IL7R | MAL1 | IFITM3 | MCL1 | IL1RN | CCL5 | C19ORF59 |
| Gene5 | KLRB1 | GZMK | S100A12 | FFAR2 | TLR4 | CYSTM1 | GZMK | IFITM1 | HSPA1B | IL7R |
| Gene6 | CCL5 | IL7R | IFITM3 | KLRB1 | KLRB1 | DDX60L | PFKFB3 | SP100 | MAL1 | HSPA1B |
| Gene7 | NAIP | TLR4 | PFKFB3 | SOD2 | SLC22A4 | FCER1A | HSPA1B | LILRA5 | TLR4 | PROK2 |
| Gene8 | PROK2 | PFKFB3 | TLR4 | CYSTM1 | HSPA1B | IL1RN | IFITM1 | MCL1 | RAB24 | ANXA3 |
| Gene9 | SELL | HSPA1B | IL1B | LRG1 | SP100 | CCR7 | IFITM3 | IFITM3 | LRG1 | MCL1 |
| Gene10 | NFIL3 | S100A12 | CD6 | IFITM1 | PLSCR1 | NAIP | C19ORF59 | TLR4 | CYSTM1 | RAB24 |
| Gene11 | CCR7 | FFAR2 | IL1RN | C19ORF59 | CCL5 | PFKFB3 | MAL1 | CD3D | IFITM1 | SOD2 |
| Gene12 | SP100 | NT5C3 | CCL5 | CSF2RB | PFKFB3 | CD3D | LILRA5 | FPR2 | GZMK | PLSCR1 |
| Gene13 | IFITM1 | SP100 | SP100 | NT5C3 | FFAR2 | SP100 | ACSL1 | GZMK | KLRB1 | FCER1A |
| Gene14 | IFITM3 | PROK2 | LRG1 | SP100 | C19ORF59 | C19ORF59 | S100A12 | ANXA3 | C19ORF59 | CSF2RB |
| Gene15 | TLR4 | IFITM3 | LILRA5 | ANXA3 | IFITM1 | CCL5 | SP100 | C19ORF59 | PFKFB3 | GZMK |
| Gene16 | GZMK | ANXA3 | SOD2 | IL1B | FCER1A | TLR4 | TLR4 | PFKFB3 | LILRA5 | DDX60L |
| Gene17 | FPR2 | SLC22A4 | DDX60L | LILRA5 | CYSTM1 | NT5C3 | PROK2 | IL7R | CD6 | MAL1 |
| Gene18 | C19ORF59 | SOD2 | IFITM1 | HSPA1B | LRG1 | FCGR1B | IL1RN | CYSTM1 | SELL | LRG1 |
| Gene19 | HSPA1B | MCL1 | IL7R | IFITM3 | IFITM3 | S100A12 | NT5C3 | CCL5 | FAIM3 | SP100 |
| Gene20 | IL7R | IFITM1 | RAB24 | TLR4 | DDX60L | HSPA1B | FFAR2 | HSPA1B | PROK2 | IFITM3 |
| Specificity | 0.74 | 0.77 | 0.77 | 0.75 | 0.74 | 0.78 | 0.77 | 0.74 | 0.75 | 0.77 |
| Sensitivity | 0.94 | 0.96 | 0.91 | 0.94 | 0.94 | 0.89 | 0.93 | 0.94 | 0.96 | 0.97 |
| AUC | 0.89 | 0.89 | 0.90 | 0.89 | 0.89 | 0.89 | 0.89 | 0.89 | 0.89 | 0.89 |

In some embodiments, the methods or kits respectively described herein use any thirty of the 40 biomarkers or genes listed in List 1.

Table 31 below shows the predictive value (Area Under Curve (AUC)) of exemplary sets of thirty biomarkers of the biomarker panel of the 40 biomarkers or genes listed in List 1 for control versus sepsis, with HPRT1/GAPDH as the housekeeping gene.

TABLE 31
Predictive value (AUC) of exemplary sets of thirty biomarkers or genes
of the biomarker panel for control versus sepsis, with HPRT1/GAPDH as the
housekeeping gene.

| Gene1 | ACSL1 | ANXA3 | C19ORF59 | CCL5 | CCR7 | CD3D | CD6 | CSF2RB |
|---|---|---|---|---|---|---|---|---|
| Gene2 | KLRB1 | LRG1 | NAIP | TLR4 | CD3D | ANXA3 | SOD2 | C19ORF59 |
| Gene3 | PFKFB3 | SLC22A4 | S100A12 | CYSTM1 | SP100 | TLR4 | IL1B | CD3D |
| Gene4 | SLC22A4 | ACSL1 | TLR4 | LILRA5 | LRG1 | C19ORF59 | C19ORF59 | FFAR2 |
| Gene5 | LRG1 | NFIL3 | PFKFB3 | C19ORF59 | LILRA5 | MAL1 | CYSTM1 | IFITM1 |
| Gene6 | IL1B | CD6 | LRG1 | PFKFB3 | RAB24 | RAB24 | KLRB1 | DDX60L |
| Gene7 | C19ORF59 | PROK2 | IL1B | MAL1 | ACSL1 | FFAR2 | MAL1 | SP100 |
| Gene8 | RAB24 | CCL5 | SOD2 | FCER1A | S100A12 | KLRB1 | SELL | TLR4 |
| Gene9 | CYSTM1 | SP100 | LILRA5 | HSPA1B | FFAR2 | IFITM3 | GZMK | CYSTM1 |
| Gene10 | IFITM3 | GZMK | IL7R | SLC22A4 | IL7R | IL7R | RAB24 | HSPA1B |
| Gene11 | HSPA1B | DDX60L | PROK2 | CD6 | IFITM3 | CYSTM1 | S100A12 | NFIL3 |
| Gene12 | SOD2 | MCL1 | CYSTM1 | IL7R | CSF2RB | NFIL3 | CCL5 | S100A12 |
| Gene13 | IL1RN | SELL | MAL1 | PLSCR1 | IL1B | ACSL1 | PLSCR1 | ACSL1 |
| Gene14 | NAIP | LILRA5 | KLRB1 | PROK2 | PROK2 | SOD2 | CD3D | FPR2 |
| Gene15 | CCR7 | S100A12 | RAB24 | ACSL1 | FCER1A | SLC22A4 | ACSL1 | FCER1A |
| Gene16 | CCL5 | HSPA1B | HSPA1B | KLRB1 | FAIM3 | LILRA5 | ANXA3 | RAB24 |
| Gene17 | FCGR1B | CSF2RB | CD3D | CSF2RB | PFKFB3 | SP100 | SP100 | SLC22A4 |
| Gene18 | PROK2 | IFITM3 | ACSL1 | ANXA3 | FPR2 | DDX60L | LRG1 | LILRA5 |
| Gene19 | FFAR2 | CD3D | FPR2 | FPR2 | C19ORF59 | HSPA1B | NT5C3 | NAIP |
| Gene20 | NFIL3 | TLR4 | CCL5 | IL1B | MAL1 | IFITM1 | PROK2 | KLRB1 |
| Gene21 | SP100 | FFAR2 | SP100 | SOD2 | KLRB1 | GZMK | IFITM1 | SOD2 |
| Gene22 | S100A12 | FAIM3 | FFAR2 | MCL1 | NFIL3 | NAIP | HSPA1B | IL7R |
| Gene23 | CD3D | SOD2 | IFITM3 | FFAR2 | DDX60L | CCL5 | SLC22A4 | IL1B |
| Gene24 | SELL | FCER1A | CSF2RB | SP100 | HSPA1B | S100A12 | FPR2 | SELL |
| Gene25 | TLR4 | PFKFB3 | SLC22A4 | NAIP | SELL | CCR7 | IFITM3 | ANXA3 |
| Gene26 | IL7R | IL1B | SELL | RAB24 | SLC22A4 | IL1B | FCER1A | PROK2 |
| Gene27 | CSF2RB | IFITM1 | IFITM1 | IFITM3 | TLR4 | FPR2 | PFKFB3 | GZMK |
| Gene28 | IFITM1 | CCR7 | FAIM3 | DDX60L | NAIP | PFKFB3 | NAIP | PFKFB3 |
| Gene29 | DDX60L | C19ORF59 | CCR7 | S100A12 | SOD2 | FCER1A | TLR4 | IFITM3 |
| Gene30 | ANXA3 | KLRB1 | DDX60L | IFITM1 | CCL5 | FAIM3 | FFAR2 | LRG1 |
| Specificity | 0.78 | 0.78 | 0.74 | 0.78 | 0.77 | 0.78 | 0.80 | 0.75 |
| Sensitivity | 0.90 | 0.93 | 0.94 | 0.90 | 0.91 | 0.91 | 0.90 | 0.91 |
| AUC | 0.91 | 0.90 | 0.91 | 0.91 | 0.91 | 0.91 | 0.90 | 0.91 |
| Gene1 | CYSTM1 | DDX60L | FAIM3 | FCER1A | FCGR1B | FFAR2 | FPR2 | GZMK |
| Gene2 | PFKFB3 | RAB24 | FPR2 | SOD2 | ACSL1 | IFITM3 | CCR7 | IL1RN |
| Gene3 | IL1B | TLR4 | PFKFB3 | ACSL1 | MAL1 | IL1RN | SP100 | SLC22A4 |
| Gene4 | PROK2 | FFAR2 | LILRA5 | FCGR1B | CCR7 | CCR7 | C19ORF59 | LRG1 |
| Gene5 | FCER1A | IFITM1 | HSPA1B | CYSTM1 | LRG1 | C19ORF59 | NAIP | IFITM1 |
| Gene6 | HSPA1B | MCL1 | FFAR2 | PFKFB3 | C19ORF59 | RAB24 | IL1B | DDX60L |
| Gene7 | SLC22A4 | HSPA1B | C19ORF59 | HSPA1B | IFITM1 | FCGR1B | TLR4 | CD3D |
| Gene8 | FFAR2 | ANXA3 | GZMK | ANXA3 | CCL5 | ACSL1 | SOD2 | ACSL1 |
| Gene9 | CCL5 | NFIL3 | IL7R | RAB24 | HSPA1B | S100A12 | ANXA3 | CSF2RB |
| Gene10 | NAIP | LILRA5 | ACSL1 | LRG1 | IL7R | KLRB1 | S100A12 | CCL5 |
| Gene11 | ACSL1 | CCR7 | CCL5 | NAIP | PFKFB3 | LILRA5 | PFKFB3 | CD6 |
| Gene12 | PLSCR1 | CCL5 | MAL1 | CCL5 | CSF2RB | IFITM1 | IFITM3 | C19ORF59 |
| Gene13 | KLRB1 | NAIP | LRG1 | TLR4 | IL1RN | CCL5 | RAB24 | KLRB1 |
| Gene14 | IFITM1 | SOD2 | DDX60L | C19ORF59 | SP100 | SOD2 | KLRB1 | FCER1A |
| Gene15 | NT5C3 | ACSL1 | SLC22A4 | FFAR2 | S100A12 | NAIP | FCGR1B | SELL |
| Gene16 | SOD2 | KLRB1 | IL1RN | CCR7 | GZMK | PROK2 | SLC22A4 | CYSTM1 |
| Gene17 | SP100 | IL1B | ANXA3 | IL1B | NAIP | HSPA1B | IL7R | CCR7 |
| Gene18 | SELL | SP100 | SP100 | PROK2 | CYSTM1 | ANXA3 | IL1RN | IFITM3 |
| Gene19 | CD6 | PFKFB3 | FCGR1B | NT5C3 | SLC22A4 | IL1B | FCER1A | LILRA5 |
| Gene20 | FPR2 | LRG1 | TLR4 | MAL1 | SOD2 | DDX60L | PROK2 | IL1B |
| Gene21 | IFITM3 | PROK2 | IL1B | DDX60L | RAB24 | SLC22A4 | LILRA5 | NAIP |
| Gene22 | FCGR1B | GZMK | FCER1A | KLRB1 | DDX60L | TLR4 | CCL5 | PROK2 |
| Gene23 | DDX60L | FCER1A | NAIP | PLSCR1 | IL1B | NT5C3 | MCL1 | MAL1 |
| Gene24 | MAL1 | IFITM3 | IFITM1 | LILRA5 | IFITM3 | IL7R | FFAR2 | IL7R |
| Gene25 | GZMK | MAL1 | RAB24 | CD6 | FAIM3 | MAL1 | CYSTM1 | FFAR2 |
| Gene26 | ANXA3 | FCGR1B | SOD2 | IFITM1 | FPR2 | SP100 | PLSCR1 | TLR4 |
| Gene27 | FAIM3 | SLC22A4 | S100A12 | IFITM3 | FFAR2 | FCER1A | FAIM3 | PFKFB3 |
| Gene28 | TLR4 | CD6 | PROK2 | MCL1 | LILRA5 | PFKFB3 | IFITM1 | RAB24 |
| Gene29 | LRG1 | CD3D | IFITM3 | FAIM3 | TLR4 | FPR2 | HSPA1B | HSPA1B |
| Gene30 | C19ORF59 | C19ORF59 | CYSTM1 | SP100 | PROK2 | CYSTM1 | DDX60L | SP100 |
| Specificity | 0.79 | 0.78 | 0.79 | 0.79 | 0.78 | 0.79 | 0.73 | 0.79 |
| Sensitivity | 0.90 | 0.91 | 0.90 | 0.90 | 0.90 | 0.90 | 0.94 | 0.94 |
| AUC | 0.90 | 0.91 | 0.91 | 0.90 | 0.90 | 0.90 | 0.90 | 0.90 |
| Gene1 | HSPA1B | IFITM1 | IFITM3 | IL1B | IL1RN | IL7R | KLRB1 | LILRA5 |
| Gene2 | CCL5 | SLC22A4 | PFKFB3 | MAL1 | NAIP | LRG1 | IL1RN | IFITM1 |
| Gene3 | SLC22A4 | FAIM3 | IL7R | CCL5 | FCGR1B | IL1B | IL7R | CYSTM1 |
| Gene4 | SELL | NAIP | IL1B | DDX60L | RAB24 | MCL1 | C19ORF59 | PFKFB3 |
| Gene5 | CCR7 | SOD2 | NAIP | NAIP | CD3D | CCL5 | CYSTM1 | S100A12 |
| Gene6 | IFITM1 | RAB24 | C19ORF59 | CSF2RB | IL1B | TLR4 | SELL | MCL1 |
| Gene7 | CD3D | C19ORF59 | DDX60L | FFAR2 | LILRA5 | CSF2RB | GZMK | IL1RN |
| Gene8 | NAIP | KLRB1 | SOD2 | LILRA5 | SP100 | SLC22A4 | FCER1A | FPR2 |
| Gene9 | LRG1 | IL1B | ANXA3 | CD6 | SLC22A4 | S100A12 | DDX60L | C19ORF59 |
| Gene10 | NFIL3 | HSPA1B | LRG1 | TLR4 | IFITM3 | KLRB1 | IFITM1 | FAIM3 |
| Gene11 | DDX60L | CCR7 | S100A12 | KLRB1 | KLRB1 | RAB24 | NFIL3 | MAL1 |
| Gene12 | IL7R | LILRA5 | NFIL3 | CCR7 | LRG1 | IFITM3 | SLC22A4 | SOD2 |
| Gene13 | ANXA3 | PLSCR1 | MCL1 | PFKFB3 | FAIM3 | SOD2 | S100A12 | KLRB1 |
| Gene14 | FAIM3 | NFIL3 | FCER1A | CYSTM1 | PFKFB3 | PFKFB3 | TLR4 | NAIP |
| Gene15 | IL1B | CYSTM1 | LILRA5 | IFITM3 | C19ORF59 | HSPA1B | SP100 | NT5C3 |
| Gene16 | SOD2 | PFKFB3 | SP100 | IFITM1 | GZMK | CCR7 | CCR7 | ACSL1 |
| Gene17 | PFKFB3 | NT5C3 | CCL5 | FCGR1B | CSF2RB | ANXA3 | ANXA3 | CD3D |
| Gene18 | IFITM3 | S100A12 | CYSTM1 | SP100 | FCER1A | DDX60L | IL1B | SP100 |
| Gene19 | GZMK | FCER1A | RAB24 | FAIM3 | SELL | FAIM3 | FAIM3 | PLSCR1 |
| Gene20 | TLR4 | TLR4 | SELL | RAB24 | PLSCR1 | FCER1A | PFKFB3 | CCL5 |
| Gene21 | ACSL1 | SP100 | ACSL1 | SELL | CYSTM1 | SP100 | CCL5 | IL1B |
| Gene22 | SP100 | CSF2RB | PROK2 | SLC22A4 | FPR2 | LILRA5 | NAIP | CD6 |
| Gene23 | FFAR2 | IL7R | FFAR2 | S100A12 | DDX60L | PROK2 | HSPA1B | IL7R |
| Gene24 | PLSCR1 | ANXA3 | TLR4 | IL1RN | IFITM1 | C19ORF59 | RAB24 | SELL |
| Gene25 | C19ORF59 | CCL5 | PLSCR1 | IL7R | IL7R | FCGR1B | FCGR1B | RAB24 |
| Gene26 | LILRA5 | FPR2 | SLC22A4 | LRG1 | HSPA1B | CD3D | ACSL1 | IFITM3 |
| Gene27 | PROK2 | MAL1 | HSPA1B | PROK2 | ANXA3 | IFITM1 | PROK2 | TLR4 |
| Gene28 | CYSTM1 | IFITM3 | IL1RN | HSPA1B | ACSL1 | FPR2 | IFITM3 | LRG1 |
| Gene29 | S100A12 | ACSL1 | IFITM1 | SOD2 | CCL5 | NAIP | LILRA5 | SLC22A4 |
| Gene30 | FCER1A | PROK2 | CCR7 | C19ORF59 | TLR4 | CYSTM1 | LRG1 | HSPA1B |
| Specificity | 0.72 | 0.79 | 0.77 | 0.79 | 0.75 | 0.77 | 0.75 | 0.75 |
| Sensitivity | 0.96 | 0.93 | 0.91 | 0.91 | 0.94 | 0.90 | 0.96 | 0.94 |
| AUC | 0.90 | 0.90 | 0.90 | 0.90 | 0.90 | 0.91 | 0.90 | 0.90 |
| Gene1 | LRG1 | MAL1 | MCL1 | NAIP | NFIL3 | NT5C3 | PFKFB3 | PLSCR1 |
| Gene2 | KLRB1 | ACSL1 | PFKFB3 | SLC22A4 | DDX60L | IFITM3 | CYSTM1 | S100A12 |
| Gene3 | IL7R | MCL1 | C19ORF59 | IL7R | MAL1 | FCER1A | NT5C3 | NT5C3 |
| Gene4 | CYSTM1 | CSF2RB | DDX60L | LRG1 | CCL5 | SELL | ACSL1 | C19ORF59 |
| Gene5 | SOD2 | S100A12 | SOD2 | CYSTM1 | CSF2RB | KLRB1 | IL1B | FCER1A |
| Gene6 | FAIM3 | NAIP | S100A12 | CD3D | ACSL1 | LILRA5 | IL1RN | SLC22A4 |
| Gene7 | S100A12 | FCER1A | CCL5 | FCER1A | IFITM3 | LRG1 | C19ORF59 | IFITM3 |
| Gene8 | FCER1A | LILRA5 | FFAR2 | IL1B | CD6 | ACSL1 | SP100 | RAB24 |
| Gene9 | CD6 | HSPA1B | CD6 | IL1RN | SLC22A4 | IFITM1 | S100A12 | CD6 |
| Gene10 | GZMK | DDX60L | FPR2 | PFKFB3 | IFITM1 | IL7R | TLR4 | SELL |
| Gene11 | IL1B | KLRB1 | IFITM3 | SP100 | FCGR1B | RAB24 | PROK2 | IFITM1 |
| Gene12 | TLR4 | CYSTM1 | NT5C3 | KLRB1 | MCL1 | TLR4 | FFAR2 | GZMK |
| Gene13 | C19ORF59 | IL7R | CCR7 | CCR7 | LRG1 | C19ORF59 | CCL5 | SOD2 |
| Gene14 | SLC22A4 | SLC22A4 | PLSCR1 | CCL5 | SP100 | ANXA3 | DDX60L | PROK2 |
| Gene15 | ACSL1 | C19ORF59 | LRG1 | GZMK | RAB24 | SLC22A4 | SELL | NAIP |
| Gene16 | CCL5 | TLR4 | IL1RN | SOD2 | SOD2 | FFAR2 | FPR2 | CCL5 |
| Gene17 | IFITM1 | LRG1 | RAB24 | DDX60L | TLR4 | PFKFB3 | SOD2 | LRG1 |
| Gene18 | PFKFB3 | IFITM1 | HSPA1B | ANXA3 | PROK2 | PROK2 | HSPA1B | FPR2 |
| Gene19 | NT5C3 | SOD2 | FCER1A | PROK2 | FFAR2 | FPR2 | GZMK | CCR7 |
| Gene20 | SELL | FPR2 | CD3D | IFITM3 | HSPA1B | FAIM3 | LILRA5 | IL1B |
| Gene21 | SP100 | PFKFB3 | KLRB1 | PLSCR1 | C19ORF59 | FCGR1B | MCL1 | IL7R |
| Gene22 | DDX60L | IL1B | FCGR1B | C19ORF59 | PFKFB3 | HSPA1B | IL7R | TLR4 |
| Gene23 | FPR2 | CCL5 | TLR4 | HSPA1B | ANXA3 | CD3D | NAIP | FFAR2 |
| Gene24 | HSPA1B | IFITM3 | PROK2 | SELL | FAIM3 | CCL5 | CCR7 | PFKFB3 |
| Gene25 | FFAR2 | FCGR1B | SP100 | S100A12 | NAIP | SOD2 | PLSCR1 | KLRB1 |
| Gene26 | IFITM3 | SP100 | IFITM1 | TLR4 | FCER1A | DDX60L | SLC22A4 | HSPA1B |
| Gene27 | CD3D | CCR7 | LILRA5 | FFAR2 | S100A12 | S100A12 | IFITM3 | ACSL1 |
| Gene28 | NAIP | GZMK | IL1B | IFITM1 | IL1B | SP100 | KLRB1 | IL1RN |
| Gene29 | MCL1 | CD6 | SLC22A4 | FPR2 | KLRB1 | NAIP | LRG1 | SP100 |
| Gene30 | PROK2 | FFAR2 | NAIP | ACSL1 | CCR7 | IL1B | IFITM1 | DDX60L |
| Specificity | 0.78 | 0.73 | 0.80 | 0.75 | 0.80 | 0.77 | 0.74 | 0.78 |
| Sensitivity | 0.91 | 0.94 | 0.90 | 0.93 | 0.87 | 0.93 | 0.94 | 0.93 |
| AUC | 0.91 | 0.91 | 0.90 | 0.91 | 0.91 | 0.91 | 0.91 | 0.91 |
| Gene1 | PROK2 | RAB24 | S100A12 | SELL | SLC22A4 | SOD2 | SP100 | TLR4 |
| Gene2 | CCL5 | CCR7 | FFAR2 | PLSCR1 | LRG1 | CYSTM1 | CD6 | MAL1 |
| Gene3 | LILRA5 | C19ORF59 | ANXA3 | GZMK | IL1B | HSPA1B | C19ORF59 | CYSTM1 |
| Gene4 | PFKFB3 | IFITM1 | IFITM3 | IFITM1 | NAIP | IL7R | TLR4 | MCL1 |
| Gene5 | ACSL1 | HSPA1B | IL1B | CCL5 | HSPA1B | ANXA3 | FAIM3 | ANXA3 |
| Gene6 | SLC22A4 | FPR2 | IFITM1 | NAIP | S100A12 | S100A12 | HSPA1B | CSF2RB |
| Gene7 | HSPA1B | LILRA5 | C19ORF59 | DDX60L | FCGR1B | LILRA5 | IL7R | PFKFB3 |
| Gene8 | C19ORF59 | FCGR1B | SOD2 | SP100 | IFITM3 | CCL5 | MAL1 | ACSL1 |
| Gene9 | IL1B | KLRB1 | FCGR1B | IL1RN | SP100 | MAL1 | ANXA3 | DDX60L |
| Gene10 | GZMK | CYSTM1 | MCL1 | TLR4 | PFKFB3 | C19ORF59 | MCL1 | IL1B |
| Gene11 | ANXA3 | FCER1A | LRG1 | FFAR2 | SELL | IFITM3 | PFKFB3 | FCER1A |
| Gene12 | KLRB1 | PROK2 | TLR4 | PROK2 | NT5C3 | FAIM3 | LRG1 | CD3D |
| Gene13 | FCGR1B | FAIM3 | KLRB1 | PFKFB3 | CD3D | TLR4 | CD3D | LRG1 |
| Gene14 | LRG1 | SP100 | HSPA1B | FAIM3 | C19ORF59 | KLRB1 | KLRB1 | SLC22A4 |
| Gene15 | FCER1A | IL7R | PLSCR1 | LRG1 | ACSL1 | IFITM1 | RAB24 | SOD2 |
| Gene16 | CYSTM1 | PFKFB3 | CCR7 | C19ORF59 | ANXA3 | NT5C3 | IFITM1 | IFITM1 |
| Gene17 | SP100 | IL1RN | GZMK | NFIL3 | PLSCR1 | PFKFB3 | FCER1A | HSPA1B |
| Gene18 | NAIP | SLC22A4 | NAIP | HSPA1B | IFITM1 | SP100 | FCGR1B | CCL5 |
| Gene19 | CCR7 | FFAR2 | CD3D | ACSL1 | KLRB1 | FPR2 | SOD2 | NFIL3 |
| Gene20 | CD6 | PLSCR1 | ACSL1 | SOD2 | MAL1 | NFIL3 | LILRA5 | IL1RN |
| Gene21 | FFAR2 | ANXA3 | CYSTM1 | S100A12 | NFIL3 | RAB24 | CCR7 | SP100 |
| Gene22 | SOD2 | SOD2 | SP100 | CD3D | FPR2 | PROK2 | DDX60L | C19ORF59 |
| Gene23 | S100A12 | IL1B | SLC22A4 | SLC22A4 | TLR4 | CSF2RB | PLSCR1 | NAIP |
| Gene24 | DDX60L | IFITM3 | RAB24 | KLRB1 | CCL5 | DDX60L | IFITM3 | IFITM3 |
| Gene25 | SELL | TLR4 | DDX6OL | FPR2 | FCER1A | ACSL1 | CYSTM1 | PROK2 |
| Gene26 | TLR4 | MCL1 | PFKFB3 | FCGR1B | FFAR2 | IL1RN | SLC22A4 | RAB24 |
| Gene27 | IFITM1 | CCL5 | CCL5 | FCER1A | FAIM3 | FCER1A | CCL5 | CD6 |
| Gene28 | RAB24 | LRG1 | IL7R | IFITM3 | SOD2 | SLC22A4 | FFAR2 | FFAR2 |
| Gene29 | IFITM3 | DDX60L | NFIL3 | LILRA5 | CYSTM1 | FCGR1B | NAIP | SELL |
| Gene30 | IL7R | SELL | NT5C3 | IL1B | GZMK | FFAR2 | S100A12 | KLRB1 |
| Specificity | 0.78 | 0.74 | 0.79 | 0.80 | 0.80 | 0.77 | 0.74 | 0.79 |
| Sensitivity | 0.91 | 0.94 | 0.89 | 0.89 | 0.89 | 0.93 | 0.96- | 0.91 |
| AUC | 0.91 | 0.91 | 0.90 | 0.90 | 0.90 | 0.91 | 0.91 | 0.90 |

FIG. 4 shows boxplots representing 6 Models (A-F) which allow the stratification of septic/non septic patients. A predetermined cut off between Sepsis/non sepsis, indicated by the respective horizontal lines, is based on a decision rule for highest total accuracy achievable. For each model a training set based on 100 samples was created (left) and a blinded test of 61 samples was used to validate the models. The Models are:
(A) using 40 genes and HPRT1 as normalization housekeeping gene.
(B) using 8 genes and HPRT1 as normalization housekeeping gene.
(C) using 40 genes and GAPDH as normalization housekeeping gene.
(D) using 8 genes and GAPDH as normalization housekeeping gene.
(E) using 40 genes and both HPRT1 and GAPDH as normalization housekeeping genes.
(F) using 11 genes and both HPRT1 and GAPDH as normalization housekeeping genes.

Table 32 below shows the predictive value (AUC) of the 6 models described above for the respective number of genes (i.e. 40 genes, 8 genes, 40 genes, 8 genes, 40 genes, 11 genes), with HPRT1/GAPDH as the housekeeping gene.

TABLE 32
Predictive value (AUC) of the 6 models for the
respective number of genes. Combined housekeeping
gene indicates both HPRT1 and GAPDH.

| No. of | | Area Under |
|---|---|---|
| genes | Models | the Curve |
| 40 | HPRT1 Housekeeping gene | 0.928 |
| 8 | HPRT1 Housekeeping gene | 0.94 |
| 40 | GAPDH Housekeeping gene | 0.927 |
| 8 | GAPDH Housekeeping gene | 0.94 |
| 40 | Combined Housekeeping gene | 0.927 |
| 11 | Combined Housekeeping gene | 0.941 |

FIG. 5 shows a boxplot representing 85 sepsis patients based on either 37 genes(A) or 14 genes(B). Weight scoring system was implemented using 2 models which allow the segregation of severe sepsis from mild sepsis.

FIG. 6 shows an average plasma protein concentration (S100Al2) in patients selected from the group consisting of control, infection, mild sepsis and severe sepsis/septic shock, indicating a correlation between severity of Sepsis and protein concentration.

Advantageously, the methods, biomarker or biomarkers and kits described can be used for the early detection and diagnosis of sepsis, and also the monitoring of patients for an improvement of treatment and outcome for such patients.

### 7. Advantageously, the Methods, Biomarker or Biomarkers and Kits Described can be Used to Identify and/or Classify a Subject or Patient as a Candidate for Sepsis Therapy. Diagnostic Kits

Detection kits may contain antibodies, aptamers, amplification systems, detection reagents (chromogen, fluorophore, etc), dilution buffers, washing solutions, counter stains or any combination thereof. Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, stratifying patient populations, diagnosis, prognosis, guiding therapeutic treatment decisions, and other applications.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Other features, benefits and advantages of the present invention not expressly mentioned above can be understood from this description by those skilled in the art.

Although the foregoing invention has been described in some detail by way of illustration and example, and with regard to one or more embodiments, for the purposes of clarity of understanding, it is readily apparent to those of ordinary skill in the art in light of the novel teachings and advantages of this invention that certain changes, variations and modifications may be made thereto without departing from the spirit or scope of the invention as described.

It would be further appreciated that although the invention covers individual embodiments, it also includes combinations of the embodiments discussed. For example, the features described in one embodiment is not being mutually exclusive to a feature described in another embodiment, and may be combined to form yet further embodiments of the invention.

### REFERENCES

1) Vallone, P. M. & Butler, J. M. AutoDimer: a screening tool for primer-dimer and hairpin structures. BioTechniques 37, 226-31 (2004).
2) Vandesompele J., De Preter K., Pattyn F., Poppe B., Van Roy N., De Paepe A. and Speleman F. (2002). Accurate normalization of real-time quantitative RT-PCR data by geometric averaging of multiple internal control genes. Genome Biology 3(7): research0034-research0034.11.
3) Kaufmann SH. Immunology's foundation: the 100-year anniversary of the Nobel Prize to Paul Ehrlich and Elie Metchnikoff. Nat Immunol. 2008 July; 9(7):705-12.
4) Segal A W. How neutrophils kill microbes. Annu Rev Immunol. 2005; 23:197-223.

1. A method of detecting or predicting sepsis in a subject, the method comprising:
i) measuring the level of at least one biomarker in a first sample isolated from the subject and
ii) comparing the level measured to a reference level of a corresponding biomarker,
wherein the at least one biomarker is selected from a group consisting of: (a) a polynucleotide comprising a nucleotide sequence set forth in any one of SEQ ID NO: 1, SEQ ID NO.: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof: (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein a difference between the level measured in the first sample and the reference level is indicative of sepsis being present in the sample.

2. The method of claim 1, wherein the presence of sepsis is determined by detecting in the subject an increase in the level of the at least one biomarker measured in the first sample, the at least one biomarker selected from a group consisting of: (a) a polynucleotide comprising a nucleotide sequence set forth in any of one of SEQ ID NO: 1, SEQ ID NO.: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, as compared to the reference level of the corresponding biomarker.

3. The method of claim 2, wherein the presence of sepsis is determined by detecting in the subject a decrease in the level of the at least one biomarker measured in the first sample, the at least one biomarker selected from a group consisting of: (a) a polynucleotide comprising a nucleotide sequence set forth in any one of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof, as compared to the reference level of the corresponding biomarker.

4. The method of claim 3, wherein the reference level is the level of the corresponding biomarker in a second sample isolated from at least one subject with no sepsis.

5. The method of claim 4, wherein the comparing step comprises applying a decision rule to determine or predict the presence or absence of sepsis in the subject.

6. A method of detecting or predicting whether a subject has one of a plurality of conditions selected from a group consisting of: control, infection, non-infected systemic inflammatory response syndrome (SIRS), mild sepsis, sever sepsis, septic shock and cryptic shock, the method comprising:
i) measuring the level of at least one biomarker in a first sample isolated from the subject; and
ii) comparing the level measured to a reference level of a corresponding biomarker,
wherein the at least one biomarker is selected from a group consisting of: (a) a polynucleotide comprising a nucleotide sequence set forth in any one of SEQ ID NO: 1, SEQ ID NO.: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one of the sequences of (a), that encodes a polypeptide comprising the corresponding amino acid sequence; and (c) a polynucleotide comprising a nucleotide sequence capable of hybridising selectively to any one of the sequences of (a), (b), or a complement thereof,
wherein the level measured in the first sample is statistically substantially similar to the reference level is indicative of whether the subject has one of the conditions.

7. The method of claim 6, wherein the reference level is the level of a corresponding biomarker in a second sample isolated from at least one subject selected from a group consisting of: a control subject, an infection positive subject, a non-infected SIRS positive subject, a mild sepsis positive subject, a sever sepsis positive subject and a cryptic shock positive subject.

8. The method of claim 7, wherein the comparing step comprises applying a decision rule to determine or predict whether the subject has one of the conditions.

9. A kit for performing the method of claim 1 the kit comprising:
i) at least one reagent capable of specifically binding to the at least one biomarker to quantify the level of the biomarker in the first sample of a subject; and
ii) a reference standard indicating the reference level of the corresponding biomarker.

10. The kit of claim 9, wherein the at least one reagent comprises at least one antibody capable of specifically binding to the at least one biomarker.

11. The kit of claim 10, further comprising at least one additional reagent capable of specifically binding at least one additional biomarker in the first sample, and a reference standard indicating a reference level of a corresponding at least one additional biomarker.

12. A kit for performing the method of claim 6, the kit comprising:
i) at least one reagent capable of specifically binding to the at least one biomarker to quantify the level of the biomarker in the first sample of a subject; and
ii) a reference standard indicating the reference level of the corresponding biomarker.

13. The kit of claim 12, wherein the at least one reagent comprises at least one antibody capable of specifically binding to the at least one biomarker.

14. The kit of claim 13, further comprising at least one additional reagent capable of specifically binding at least one additional biomarker in the first sample, and a reference standard indicating a reference level of a corresponding at least one additional biomarker.

15-18. (canceled)

19. A method of detecting or predicting sepsis in a subject, the method comprising:
i) measuring the level of at least one biomarker in a first sample isolated from the subject; and
ii) comparing the level measured to a reference level of a corresponding biomarker,
wherein the at least one biomarker is selected from a group consisting of: (a) a polynucleotide comprising a nucleotide sequence set forth in any one or more, and in any combination, of SEQ ID NO: 1, SEQ ID NO.: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or a fragment, homologue, variant or derivative thereof; (b) a polynucleotide comprising a nucleotide sequence set forth in any one or more, and in any combination, of the sequences of (a), that encodes a polypeptide comprising the corresponding amino acid sequence; and (c) a polynucleotide comprising a nucleotide sequence capable of hybridising selectively to any one or more of the sequences of (a), (b), or a complement thereof,
wherein a difference between the level measured in the first sample and the reference level is indicative of sepsis being present in the first sample.

20. (canceled)
